(19) United States
(12) Patent Application Publication
(10) Pub. No.: US 2026/0042835 A1
(43) Pub. Date: Feb. 12, 2026

(54) METHODS FOR TREATING CHRONIC MYELOMONOCYTIC LEUKEMIA WITH ANTI-ILT3 ANTIBODIES

(71) Applicant: Merck Sharp & Dohme LLC, Rahway, NJ (US)

(72) Inventors: Mei Chen, Audubon, PA (US); Cai Wu, Somerset, NJ (US); Daping Zhang, Kirkland, WA (US); Jie Zhang-Hoover, Lexington, MA (US); Ghayas Issa, Houston, TX (US)

(73) Assignee: Merck Sharp & Dohme LLC, Rahway, NJ (US)

(21) Appl. No.: 18/998,702

(22) PCT Filed: Jul. 27, 2023
(86) PCT No.: PCT/US2023/028847

§ 371 Date: Jan. 27, 2025

Related U.S. Application Data
(60) Provisional application No. 63/369,705, filed on Jul. 28, 2022.

(51) Int. Cl. C07K 16/28; A61K 39/00; A61P 35/02

(52) U.S. Cl. CPC C07K 16/2803; A61P 35/02; A61K 2039/505; A61K 2039/545; C07K 2317/73

(57) ABSTRACT

This disclosure relates to methods for treating cancer in a subject identified as having chronic myelomonocytic leukemia (CMML), comprising administering an anti-ILT3 antigen binding protein, or antigen binding fragment to the patient every three weeks (Q3W).

### CROSS-REFERENCE TO RELATED APPLICATIONS

This application claims the benefit of U.S. Provisional Patent Application Ser. No. 63/369,705 filed Jul. 28, 2022, the entire contents of which are incorporated by reference herein.

### REFERENCE TO SEQUENCE LISTING SUBMITTED ELECTRONICALLY

The contents of the electronic sequence listing (25564-WO-SEQLIST-12May 2023.xml; Size: 260,840 bytes and Date of Creation May 12, 2023) are herein incorporated by reference in their entirety.

### FIELD

This disclosure relates to methods for treating cancer in a subject comprising administering an anti-ILT3 antigen binding protein, including an antibody or antigen binding fragment to the subject.

### BACKGROUND OF THE INVENTION

Chronic myelomonocytic leukemia (CMML) is a myeloid neoplasm characterized by dysplasia, abnormal production and accumulation of monocytic cells with an elevated risk of transforming into acute leukemia. It is characterized by 1) persistent monocytosis>1×10⁹/L in the peripheral blood with monocytes≥10% of white blood cell count; 2) along with dysplastic features in the bone marrow, lack of Philadelphia chromosome and BCR-ABL 1 fusion gene; 3) no rearrangement of PDGFRA or PDGFRB; 4) fewer than 20% blasts in peripheral blood and bone marrow; and 5) dysplasia involving one or more myeloid lineages [Arber, D. A., et al. 2016] [Orazi, A., et al. 2008].

Due to renewed evidence demonstrating clinical, morphological and molecular differences, the 2016 WHO classification recommended categorization of CMML into “proliferative” (MPN-CMML) and “dysplastic” (MDS-CMML) subtypes based on a white blood cell count of ≥13×10⁹/L for MPN-CMML [Arber, D. A., et al. 2016]. In addition, based on peripheral blood and bone marrow blast percentage, CMML can be classified into 3 subcategories; CMML-0, a category for cases with <2% blasts in peripheral blood and <5% blasts in bone marrow; CMML-1 for cases with 2% to 4% blasts in peripheral blood and/or 5% to 9% blasts in bone marrow; and CMML-2 for cases with 5% to 19% blasts in peripheral blood, 10% to 19% in bone marrow, and/or when any Auer rods are present [Arber, D. A., et al. 2016].

Therapeutic approaches in CMML have generally been the model for treating the other MDS/MPN, with hypomethylating agent treatment for intermediate- and higher-risk patients and using these agents as a bridge to allogeneic hematopoietic cell transplantation for those patients deemed to be transplant eligible [National Comprehensive Cancer Network 2022][Patnaik, M. M. 2018]. Allogeneic stem cell transplantation remains the only curative option for patients with CMML. This modality is however fraught with complications including, acute and chronic GVHD, non-relapse mortality and posttransplant disease relapse [Patnaik, M. M. 2018]. Reported median survival time is 20 to 40 months and progression to AML occurs in approximately 15% to 30% [Orazi, A., et al. 2017][Geissler, K. 2021]. These patients have strong unmet medical need.

Acute myeloid leukemia (AML) is a heterogenous hematologic malignancy characterized by the clonal expansion of myeloid blasts in the bone marrow, peripheral blood, and potentially other tissues [Dohner, H., et al. 2015]. AML is the most common form of adult acute leukemia in the US [Carter, J. L., et al. 2020] with a median age of around 68 years at the initial diagnosis [Shallis, R. M., et al. 2019]. In 2021 in the US, the estimated number of new AML cases is ˜20,240 and the estimated number of deaths due to AML is ˜11,400 [Siegel, R. L., et al. 2021]. Despite increasing understanding of the underlying biology of AML and the development of several new therapies, the 5-year relative survival rate remains low, at about 26% based on 2021 estimate from SEER [American Cancer Society 2021].

Immunoglobulin-like transcript 3 (ILT3), designated CD85k and also known as Leukocyte Immunoglobulin-Like Receptor subfamily B member 4 (LILRB4) and Leukocyte Immunoglobulin-like Receptor 5 (LIR-5), is a type I membrane protein that contains cytoplasmic immunoreceptor tyrosine-based inhibition motif (ITIM) motifs and is involved in the down-regulation of immune responses (Cella et al., J Exp Med. 185 (10): 1743-51 (1997); Samaridis et al., Eur J Immunol. 27 (3): 660-665 (1997). Expression of ILT3 is up-regulated on tolerogenic dendritic cells. This gene is a member of the leukocyte immunoglobulin-like receptor (LIR) family, which is found in a gene cluster at chromosomal region 19q13.4. The encoded protein belongs to the subfamily B class of LIR receptors, which contain two or four extracellular immunoglobulin domains, a transmembrane domain, and two to four ITIMs.

Expression of ILT3 has been reported on dendritic cells, monocytic myeloid cells, macrophages, progenitor mast cells, endothelial cells and osteoclasts. The expression of ILT3 on myeloid cells and dendritic cells is thought to be involved in immune suppression and antigen-specific immune tolerance and is considered to be contributing to the immunosuppressive tumor microenvironments in various human cancers [Kang, X., et al. 2016].

Further evaluation by Li et al. [Li, Z., et al. 2020] suggested that the intracellular ITIM domain of activated ILT3 recruits SHP-2, which activates NFκB. Activation of NFκB results in regulation of downstream effectors including uPAR and ARG1, leading to inhibition of T-cell proliferation and infiltration of AML cells into tissues. Gui et al. developed a humanized antibody to ILT3 h128-3. Disrupting ILT3/APOE interaction using h128-3 could reverse T-cell suppression and block AML development in mouse models [Gui, X., et al. 2019].

The ILT3 pathway may be a key regulatory element responsible for the induction and maintenance of tumor immune tolerance. Inhibitors of ILT3 may provide an innovative and tractable method to treat CMML.

### SUMMARY OF THE INVENTION

In a first aspect, the present disclosure provides a method for treating chronic myelomonocytic leukemia (CMML) in a subject comprising administering to a subject a therapeutically effective dose of a pharmaceutical composition comprising an anti-ILT3 antigen binding protein or antigen binding fragment and a pharmaceutically acceptable excipient.

In some embodiments of the first aspect, the subject has a confirmed diagnosis of CMML. In some embodiments, the subject has confirmed CMML as determined by a) persistent monocytosis>1×10⁹/L in the peripheral blood with monocytes≥10% of white blood cell count; b) dysplastic features in the bone marrow, lack of Philadelphia chromosome and BCR-ABL 1 fusion gene; c) no rearrangement of PDGFRA or PDGFRB; d) fewer than 20% blasts in peripheral blood and bone marrow; and e) dysplasia involving one or more myeloid lineages. In some embodiments, the subject is a human.

In some embodiments, the anti-ILT3 antigen-binding protein or antigen-binding fragment is an anti-ILT3 antibody or antigen-binding fragment. In some embodiments, the anti-ILT3 antigen binding protein or antigen binding fragment comprises: a heavy chain (HC) wherein the heavy chain variable domain (VH) comprises a heavy chain complementarity determining region (HC-CDR) 3 having an amino acid sequence selected from the group consisting of SEQ ID NO: 15, 42, 50, 58, 66, 74, 82, 90, and 98, or having an amino acid sequence that has 3, 2, or 1 differences with an amino acid sequence selected from the group consisting of SEQ ID NO: 15, 42, 50, 58, 66, 74, 82, 90, and 98.

In some embodiments, the anti-ILT3 antibody or antigen binding fragment comprises: (a) a heavy chain (HC) having a variable domain (VH) comprising a variable domain complementarity determining region (HC-CDR) 1 having the amino acid sequence set forth in SEQ ID NO: 10, 40, 48, 56, 64, 72, 80, 88, or 96; an HC-CDR2 having the amino acid sequence set forth in SEQ ID NO: 11, 41, 48, 57, 64, 73, 81, 89, or 97; and an HC-CDR3 having the amino acid sequence set forth in SEQ ID NO: 16, 42, 50, 58, 66, 74, 82, 90, or 98; and, variants thereof wherein one or more of the HC-CDRs has one, two, or three amino acid substitutions, additions, deletions, or combinations thereof; and (b) a light chain (LC) having variable domain (VL) comprising a variable domain complementarity determining region (LC-CDR) 1 having the amino acid sequence set forth in SEQ ID NO: 20, 43, 51, 59, 67, 75, 83, 91, or 99; an LC-CDR2 having the amino acid sequence set forth in SEQ ID NO: 36, 44, 52, 60, 68, 76, 84, 92, or 100; and an LC-CDR3 having the amino acid sequence set forth in SEQ ID NO: 37, 45, 53, 61, 69, 77, 85, 93, or 101; and, variants thereof wherein one or more of the LC-CDRs has one, two, or three amino acid substitutions, additions, deletions, or combinations thereof.

In some embodiments, (a) the HC-CDR1 has the amino acid sequence set forth in SEQ ID NO: 10; the HC-CDR2 has the amino acid sequence set forth in SEQ ID NO: 12, 13, or 14; the HC-CDR3 has the amino acid sequence set forth in SEQ ID NO: 16; and (b) the LC-CDR1 has the amino acid sequence set forth in SEQ ID NO: 27, 28, 29, 30, 31, 32, 33, 34, or 35; the LC-CDR2 has the amino acid sequence set forth in SEQ ID NO: 36; and, the LC-CDR3 has the amino acid sequence set forth in SEQ ID NO: 37.

In some embodiments, (a) the HC-CDR1 has the amino acid sequence set forth in SEQ ID NO: 10; the HC-CDR2 has the amino acid sequence set forth in SEQ ID NO: 13; and the HC-CDR3 has the amino acid sequence set forth in SEQ ID NO: 16; and (b) the LC-CDR1 has the amino acid sequence set forth in SEQ ID NO: 34; the LC-CDR2 has the amino acid sequence set forth in SEQ ID NO: 36; and, the LC-CDR3 has the amino acid sequence set forth in SEQ ID NO: 37.

In some embodiments, the VH comprises a framework selected from the group consisting of human V_{H}1, V_{H}2, V_{H}3, V_{H}4, V_{H}5, and V_{H}6, and variants thereof having 1, 2, 3, 4, 5, 6, 7, 8, 9, or 10 amino acid substitutions, additions, deletions, or combinations thereof, and, the VL comprises a framework selected from the group consisting of human V_{K}1, V_{K}2, V_{K}3, V_{K}4, V_{K}5, V_{K}6, V_{λ}1, V_{λ}2, V_{λ}3, V_{λ}4, V_{λ}5, V_{λ}6, V_{λ}7, V_{λ}8, V_{λ}9, and V_{λ}10, and variants thereof having 1, 2, 3, 4, 5, 6, 7, 8, 9, or 10 amino acid substitutions, additions, deletions, or combinations thereof.

In some embodiments, the antibody comprises an HC having a human IgG1, IgG2, IgG3, or IgG4 HC constant domain or variant thereof having 1, 2, 3, 4, 5, 6, 7, 8, 9, or 10 amino acid substitutions, additions, deletions, or combinations thereof compared to the amino acid sequence of the native IgG1, IgG2, IgG3, or IgG4 isotype constant domain.

In some embodiments, the antibody comprises an LC having a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda light chain constant domain.

In some embodiments, the antibody comprises: (i) a VH having a framework selected from human V_{H}1, V_{H}2, V_{H}3, V_{H}4, V_{H}5, and V_{H}6 and a human IgG1 or IgG4 HC constant domain or variant thereof comprising 1, 2, 3, 4, 5, 6, 7, 8, 9, or 10 amino acid substitutions, additions, deletions, or combinations thereof compared to the amino acid sequence of the native IgG1 or IgG4 isotype HC constant domain; and, (ii) a VL having a framework selected from human V_{K}1, V_{K}2, V_{K}3, V_{K}4, V_{K}5, V_{K}6, V_{λ}1, V_{λ}2, V_{λ}3, V_{λ}4, V_{λ}5, V_{λ}6, V_{λ}7, V_{λ}8, V_{λ}9, and V_{λ}10 and a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda LC constant domain.

In some embodiments, the antibody or antigen binding fragment comprises a V_{H} and a V_{L} having the amino acid sequences set forth in SEQ ID NO: 8 and SEQ ID NO: 9, respectively; SEQ ID NO:38 and SEQ ID NO: 39, respectively; SEQ ID NO: 46 and SEQ ID NO: 47, respectively; SEQ ID NO: 54 and SEQ ID NO: 55, respectively; SEQ ID NO: 62 and SEQ ID NO: 63, respectively; SEQ ID NO: 70 and SEQ ID NO: 71, respectively; SEQ ID NO: 78 and SEQ ID NO: 79, respectively; SEQ ID NO: 86 and SEQ ID NO: 87, respectively; or SEQ ID NO:94 and SEQ ID NO: 95, respectively.

In some embodiments, the antibody or antigen binding fragment comprises a V_{H} having the amino acid sequence set forth in SEQ ID NO: 110, 111, 112, 116, 117, or 118 and a V_{L} having the amino acid sequence set forth in SEQ ID NO: 119, 120, 121, 122, 123, 124, 125, 126, 127, 128, 129, 130, 131, 132, 133, or 134.

In some embodiments, the antibody or antigen binding fragment comprises a VH having the amino acid sequence set forth in SEQ ID NO: 111 and a VL having the amino acid sequence set forth in SEQ ID NO: 133.

In some embodiments, the antibody comprises a heavy chain (HC) constant domain comprising the amino acid sequence set forth in SEQ ID NO: 2, 3, 4, 5, or 6.

In some embodiments, the antibody comprises a light chain (LC) constant domain comprising the amino acid sequence set forth in SEQ ID NO: 7.

In some embodiments, the antibody comprises a heavy chain (HC) comprising the amino acid sequence of SEQ ID NO: 135, 136, 137, 141, 142, 143, 160, 161, 162, 163, 167, 168, 169, 170, 171, 175, 176, 177, 178, 179, 180, 184, 185, or 186.

In some embodiments, the antibody comprises a light chain (LC) comprising the amino acid sequence set forth in SEQ ID NO: 144, 145, 146, 147, 148, 149, 150, 151, 152, 153, 154, 155, 156, 157, 158, or 159.

In some embodiments, the antibody comprises a heavy chain (HC) comprising the amino acid sequence set forth in SEQ ID NO: 136 and a light chain (LC) comprising the amino acid sequence set forth in SEQ ID NO: 158, and variants thereof wherein the HC lacks a C-terminal Lysine residue or a C-terminal glycine-lysine.

In some embodiments, the therapeutically effective amount of the anti-ILT3 antigen binding protein or antigen binding fragment is between about 7.5 mg and about 2250 mg. In some embodiments, the therapeutically effective amount of anti-ILT3 antigen binding protein or antigen binding fragment selected from the group consisting of: 7.5 mg; 25 mg; 75 mg; 225 mg; 750 mg; and 2250 mg. In some embodiments, the therapeutically effective amount of anti-ILT3 antigen binding protein or antigen binding fragment is 7.5 mg. In some embodiments, the therapeutically effective amount of anti-ILT3 antigen binding protein or antigen binding fragment is 25 mg. In some embodiments, the therapeutically effective amount of anti-ILT3 antigen binding protein or antigen binding fragment is 75 mg. In some embodiments, the therapeutically effective amount of anti-ILT3 antigen binding protein or antigen binding fragment is 225 mg. In some embodiments, the therapeutically effective amount of anti-ILT3 antigen binding protein or antigen binding fragment is 750 mg. In some embodiments, the therapeutically effective amount of anti-ILT3 antigen binding protein or antigen binding fragment is 2250 mg.

In some embodiments, the anti-ILT3 antibody or antigen binding fragment are administered every three weeks (Q3W) of a 21-day cycle.

In some embodiments, the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: (a) the HC-CDR1 comprises the amino acid sequence set forth in SEQ ID NO: 10; the HC-CDR2 comprises the amino acid sequence set forth in SEQ ID NO: 12; the HC-CDR3 comprises the amino acid sequence set forth in SEQ ID NO: 16; the LC-CDR1 comprises the amino acid sequence set forth in SEQ ID NO: 36; the LC-CDR2 comprises the amino acid sequence set forth in SEQ ID NO: 36; and the LC-CDR3 comprises the amino acid sequence set forth in SEQ ID NO: 37; (b) the HC-CDR1 has the amino acid sequence set forth in SEQ ID NO: 10; the HC-CDR2 has the amino acid sequence set forth in SEQ ID NO: 13; the HC-CDR3 has the amino acid sequence set forth in SEQ ID NO: 16; the LC-CDR1 has the amino acid sequence set forth in SEQ ID NO: 32; the LC-CDR2 has the amino acid sequence set forth in SEQ ID NO: 36; and, the LC-CDR3 has the amino acid sequence set forth in SEQ ID NO: 37; (c) the HC-CDR1 has the amino acid sequence set forth in SEQ ID NO: 10; the HC-CDR2 has the amino acid sequence set forth in SEQ ID NO: 14; the HC-CDR3 has the amino acid sequence set forth in SEQ ID NO: 16; the LC-CDR1 has the amino acid sequence set forth in SEQ ID NO: 33; the LC-CDR2 has the amino acid sequence set forth in SEQ ID NO: 36; and, the LC-CDR3 has the amino acid sequence set forth in SEQ ID NO: 37; (d) the HC-CDR1 has the amino acid sequence set forth in SEQ ID NO: 10; the HC-CDR2 has the amino acid sequence set forth in SEQ ID NO: 13; the HC-CDR3 has the amino acid sequence set forth in SEQ ID NO: 16; the LC-CDR1 has the amino acid sequence set forth in SEQ ID NO: 34; the LC-CDR2 has the amino acid sequence set forth in SEQ ID NO: 36; and, the LC-CDR3 has the amino acid sequence set forth in SEQ ID NO: 37; or (e) the HC-CDR1 has the amino acid sequence set forth in SEQ ID NO: 10; the HC-CDR2 has the amino acid sequence set forth in SEQ ID NO: 12; the HC-CDR3 has the amino acid sequence set forth in SEQ ID NO: 16; the LC-CDR1 has the amino acid sequence set forth in SEQ ID NO: 35; the LC-CDR2 has the amino acid sequence set forth in SEQ ID NO: 36; and, the LC-CDR3 has the amino acid sequence set forth in SEQ ID NO: 37.

In some embodiments, the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 comprises the amino acid sequence set forth in SEQ ID NO: 10; the HC-CDR2 comprises the amino acid sequence set forth in SEQ ID NO: 12; the HC-CDR3 comprises the amino acid sequence set forth in SEQ ID NO: 16; the LC-CDR1 comprises the amino acid sequence set forth in SEQ ID NO: 31; the LC-CDR2 comprises the amino acid sequence set forth in SEQ ID NO: 36; and the LC-CDR3 comprises the amino acid sequence set forth in SEQ ID NO: 37.

In some embodiments, the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 10; the HC-CDR2 has the amino acid sequence set forth in SEQ ID NO: 13; the HC-CDR3 has the amino acid sequence set forth in SEQ ID NO: 16; the LC-CDR1 has the amino acid sequence set forth in SEQ ID NO: 32; the LC-CDR2 has the amino acid sequence set forth in SEQ ID NO: 36; and, the LC-CDR3 has the amino acid sequence set forth in SEQ ID NO: 37. In some embodiments, the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 10; the HC-CDR2 has the amino acid sequence set forth in SEQ ID NO: 14; the HC-CDR3 has the amino acid sequence set forth in SEQ ID NO: 16; the LC-CDR1 has the amino acid sequence set forth in SEQ ID NO: 33; the LC-CDR2 has the amino acid sequence set forth in SEQ ID NO: 36; and, the LC-CDR3 has the amino acid sequence set forth in SEQ ID NO: 37.

In some embodiments, the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 10; the HC-CDR2 has the amino acid sequence set forth in SEQ ID NO: 13; the HC-CDR3 has the amino acid sequence set forth in SEQ ID NO: 16; the LC-CDR1 has the amino acid sequence set forth in SEQ ID NO: 34; the LC-CDR2 has the amino acid sequence set forth in SEQ ID NO: 36; and, the LC-CDR3 has the amino acid sequence set forth in SEQ ID NO: 37.

In some embodiments, the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 10; the HC-CDR2 has the amino acid sequence set forth in SEQ ID NO: 12; the HC-CDR3 has the amino acid sequence set forth in SEQ ID NO: 16; the LC-CDR1 has the amino acid sequence set forth in SEQ ID NO: 35; the LC-CDR2 has the amino acid sequence set forth in SEQ ID NO: 36; and, the LC-CDR3 has the amino acid sequence set forth in SEQ ID NO: 37.

In some embodiments, the anti-ILT3 antigen binding protein or antigen binding fragment comprises: (a) a heavy chain of SEQ ID NO: 140 and a light chain of SEQ ID NO: 149; (b) a heavy chain of SEQ ID NO: 146 and a light chain of SEQ ID NO: 151; (c) a heavy chain of SEQ ID NO: 141 and a light chain of SEQ ID NO: 150; (d) a heavy chain of SEQ ID NO: 141 and a light chain of SEQ ID NO: 163; or (e) a heavy chain of SEQ ID NO: 144 and a light chain of SEQ ID NO: 150.

In some embodiments, the anti-ILT3 antigen binding protein or antigen binding fragment comprises a heavy chain of SEQ ID NO: 140 and a light chain of SEQ ID NO: 149. In some embodiments, the anti-ILT3 antigen binding protein or antigen binding fragment comprises a heavy chain of SEQ ID NO: 146 and a light chain of SEQ ID NO: 151. In some embodiments, the anti-ILT3 antigen binding protein or antigen binding fragment comprises a heavy chain of SEQ ID NO: 141 and a light chain of SEQ ID NO: 150. In some embodiments, the anti-ILT3 antigen binding protein or antigen binding fragment comprises a heavy chain of SEQ ID NO: 141 and a light chain of SEQ ID NO: 163. In some embodiments, the anti-ILT3 antigen binding protein or antigen binding fragment comprises a heavy chain of SEQ ID NO: 144 and a light chain of SEQ ID NO: 150.

In a second aspect, the disclosure provides a pharmaceutical composition comprising 0.02 mg to 2250 mg of an anti-ILT3 antigen binding protein or antigen binding fragment and a pharmaceutically acceptable excipient for use in the methods of any one of the above aspects and embodiments.

In another aspect, the disclosure provides the use of a pharmaceutical composition comprising 0.02 mg to 2250 mg of an anti-ILT3 antigen binding protein or antigen binding fragment and a pharmaceutically acceptable excipient in the manufacture of a medicament for use in any of the methods disclosed herein.

The summary of the technology described above is non-limiting and other features and advantages of the technology will be apparent from the following detailed description, and from the claims.

### BRIEF DESCRIPTION OF THE FIGURES

FIG. 1 shows a dot plot quantitating and comparing the percentage of 10 clusters of myeloid cell phenotypes between the 52B8 and hIgG4 isotype treatments. Filled circles represent cells treated with antibody 52B8, and empty circles are cells treated with control antibody (human IgG4). Cluster 1 represents the monocytic myeloid cell phenotype and Cluster 4 represents tumor blast phenotype.

FIG. 2A shows a graph of the mean fluorescence and standard error of the mean (SEM) for MV-4-11 luc cells inoculated in a humanized mouse, treated with anti-ILT3 antibody 52B8 or control human IgG4 antibody (hIgG4), and then harvested from bone marrow at days 7, 14, 21, 28, 35 following inoculation. Filled circles are cells treated with 52B8 at 10 mpk i.p. QW, open circles represent cells treated with hIgG4. FIG. 2B shows a dot plot of the percentage of MV-4-11 luc cells in bone marrow cells from the 52B8 antibody- and control antibody-treated groups.

FIGS. 3A and 3B show bar graphs of IFN-γ expression in human CD8⁺ T cells from two different human donors, following in vitro treatment with control antibody (10 μg/mL hIgG4) or various concentrations of 52B8 antibody (10 μg/mL, 1 μg/mL, and 0.1 μg/mL).

FIG. 4 is a schematic drawing of a clinical study design for treating AML and CMML patients with doses of anti-ILT3 antibody.

### DETAILED DESCRIPTION OF THE DISCLOSURE

### Definitions & Abbreviations

As used throughout the specification and appended claims, the following abbreviations apply:
ADA antidrug antibodies
AE adverse event
ALT alanine aminotransferase
AML acute myeloid leukemia
ANC absolute neutrophil count
APOE apolipoprotein E
AST aspartate aminotransferase
ATD accelerated titration design
BCG bacillus Calmette-Guerin
BLI bioluminescent imaging
C1D1 Cycle 1 Day 1
CBC complete blood count
CDR complementarity determining region
CDRH complementarity determining region in a heavy chain variable domain
CDRL complementarity determining region in a light chain variable domain
CMML chronic myelomonocytic leukemia
CNS central nervous system
CONSORT Consolidated Standards of Reporting Trials
CL clearance
CrCl creatinine clearance
CR complete remission or complete response
CRF Case Report Form
CRi complete remission without hematologic recovery
CSF Cerebrospinal fluid
NCI CTCAE 5.0 National Cancer Institute Common Terminology Criteria for Adverse Events, Version 5.0
DILI drug-induced liver injury
DL dose level
DLT dose-limiting toxicity
DNA deoxyribonucleic acid
ECI event of clinical interest
eCRF electronic Case Report Form
ECOG Eastern Cooperative Oncology Group
ELN European Leukemia Net
FISH fluorescence in situ hybridization
FR framework region
GCP Good Clinical Practice
G-CSF granulocyte-colony stimulating factor
GFR glomerular filtration rate
GM-CSF granulocyte-macrophage colony stimulating factor
GVHD graft versus host disease
HBsAg hepatitis B surface antigen
HBV hepatitis B virus
HCV hepatitis C virus
Hgb hemoglobin
HIV human immunodeficiency virus
IDH isocitrate dehydrogenase
Ig immunoglobulin
ILT3 immunoglobulin-like transcript 3
IP intraperitoneal
IV intravenous
IVRS interactive voice response system
IWRS integrated web response system
LILRB leukocyte immunoglobulin-like receptor subfamily B
luc luciferase
mAb monoclonal antibody
MDSC myeloid-derived suppressor cell
MDS/MPN myelodysplastic syndromes/myeloproliferative neoplasms
MLFS morphologic leukemia-free state
mpk milligrams per kilogram
MPN-SAF myeloproliferative neoplasm symptom assessment form
MTD maximum tolerated dose
mTPI modified Toxicity Probability Interval
NCI National Cancer Institute
NYHA New York Heart Association
OR objective response
OTC over-the-counter
PK pharmacokinetic
PR partial remission
Q3W every 3 weeks
RNA ribonucleic acid
RP2D recommended Phase 2 dose
R/R relapsed/refractory
SAE serious adverse event
SCT stem cell transplant
SEM standard error of the mean
SGOT serum glutamic oxaloacetic transaminase
SGPT serum glutamic-pyruvic transaminase
sILT3 soluble ILT3; part or all of an ILT3 extracellular domain that is not membrane bound
t½ half-life
TI transfusion independence
TLS Tumor Lysis Syndrome
TSS total symptom score
ULN upper limit of normal
VH immunoglobulin heavy chain variable region or domain
VL immunoglobulin light chain variable region or domain
WBC white blood cell
WHO World Health Organization
wk week
WOCBP woman/women of childbearing potential

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

Reference to “or” indicates either or both possibilities unless the context clearly dictates one of the indicated possibilities. In some cases, “and/or” was employed to highlight either or both possibilities.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

The term “about”, when modifying the quantity (e.g., mg) of a substance or composition, or the value of a parameter characterizing a step in a method, or the like, refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±10%.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, an anti-ILT3 antigen binding fragment that consists essentially of a recited amino acid sequence may also include one or more amino acids, including substitutions of one or more amino acid residues, which do not materially affect the properties of the binding compound.

“Administration” and “treatment,” as they apply to an animal, human, experimental subject, cell, tissue, organ, or biological fluid, refer to contact of an exogenous pharmaceutical, therapeutic, diagnostic agent, or composition to the animal, human, subject, cell, tissue, organ, or biological fluid. “Treat” or “treating” CMML, as used herein, means to administer an anti-ILT3 antigen binding protein (e.g., an antibody) or antigen-binding fragment, to a subject having CMML, to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, halting or delaying the growth of a tumor or blood cancer or progression of disease associated with ILT-3, ameliorating or abrogating the clinical manifestations of ILT-3-related disease, reducing the severity or duration of the clinical symptoms of ILT-3-related disease such as cancer, prolonging the survival of a patient relative to the expected survival in a similar untreated patient, and inducing complete or partial remission of a cancerous condition or other ILT-3-related disease.

Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the treatment methods, compositions and uses of the present invention may not be effective in achieving a positive therapeutic effect in every patient, it should do so in a statistically significant number of subjects as determined by any statistical test known in the art such as the Student's t-test, the chi2-test, the U-test according to Mann and Whitney. Positive therapeutic effects in a leukemia such as CMML may include measuring reductions in the number of CMML cells in a bone marrow sample. Detection of CMML cells may be accomplished using flow cytometric methods to identify cellular biomarkers, detection of RNA transcripts associated with CMML cells.

The terms “effective amount”, “therapeutically effective amount”, and “therapeutically effective dose” refer to an amount of an anti-ILT3 antigen binding protein or antigen binding fragment (e.g. an anti-ILT3 antibody) of the invention that, when administered alone or in combination with an additional therapeutic/prophylactic agent to a cell, tissue, or subject, is effective to prevent or cause a measurable improvement in one or more symptoms of the disease or condition being treated, e.g., CMML, as disclosed herein. An effective dose further refers to that amount of the anti-ILT3 antigen binding protein or antigen binding fragment sufficient to result in at least partial prevention or amelioration of symptoms of the disease or condition being treated, either alone or in combination with another compound.

The antigen binding proteins or antigen binding proteins disclosed herein may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound or compounds disclosed herein depend on the pharmacokinetic properties of that compound or compounds, such as absorption, distribution and half-life which can be determined by a skilled artisan. In addition, suitable dosing regimens, including the duration such regimens are administered, for a compound or compounds disclosed herein depend on the disease or condition being treated, the severity of the disease or condition, the age and physical condition of the subject being treated, the medical history of the subject being treated, the nature of concurrent therapy, the desired therapeutic effect, and like factors within the knowledge and expertise of the skilled artisan. It will be further understood by such skilled artisans that suitable dosing regimens may require adjustment given an individual subject's response to the dosing regimen or over time as the individual subject needs change. Typical daily dosages may vary depending upon the particular route of administration chosen.

The term “subject” (alternatively referred to as “patient” or “individual” herein) refers to a mammal (e.g., rat, mouse, dog, cat, rabbit) capable of being treated with the methods and compositions of the invention, most preferably a human. In some embodiments, the subject is an adult subject. In other embodiments, the subject is a pediatric subject.

“Biologic agent” or “biotherapeutic agent” means a biological molecule, such as an antibody or fusion protein, that blocks ligand/receptor signaling in any biological pathway that supports tumor maintenance and/or growth or suppresses the anti-tumor immune response. “Biologic therapy” or “biological therapy” refers to a cancer treatment using a protein.

“Targeted agent” or “targeted therapeutic agent” refers to a therapeutic agent (either a small molecule or protein) that affects a specific protein type or class of proteins that are associated with tumor cell growth or spread in a patient's body.

“Systemic therapy” refers to a cancer treatment using therapeutic agents injected in a patient's bloodstream that affect cells throughout the patient's body, including chemotherapy, biological therapy, and targeted therapy.

“Chemotherapy” refers to an anti-cancer treatment using one or more “chemotherapeutic agents”. A “chemotherapeutic agent” is a drug used to treat CMML, including, but not limited to: cytarabine (also called cytosine arabinoside or ara-C); an anthracycline, e.g., daunorubicin (also called daunomycin) or idarubicin; cladribine (2-CdA); fludarabine; mitoxantrone; etoposide (VP-16); 6-thioguanine (6-TG); hydroxyurea; corticosteroids, e.g., prednisone or dexamethasone; methotrexate (MTX); 6-mercaptopurine (6-MP); azacitidine; and decitabine.

As used herein, the term “neoplastic disease” is characterized by malignant growth or in disease states characterized by benign hyperproliferative and hyperplastic cells. The common medical meaning of the term “neoplasia” refers to “new cell growth” that results as a loss of responsiveness to normal growth controls, e.g., neoplastic cell growth.

As used herein, the terms “hyperproliferative”, “hyperplastic”, malignant” and “neoplastic” are used interchangeably, and refer to those cells in an abnormal state or condition characterized by rapid proliferation or neoplasia. The terms are meant to include all types of hyperproliferative growth, hyperplastic growth, cancerous growths or oncogenic processes, metastatic tissues or malignantly transformed cells, tissues, or organs, irrespective of histopathologic type or stage of invasiveness. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias may include “tumors,” which may be either benign, premalignant or malignant. Extramedullary leukemia (EML) is referred to as granulocytic sarcoma, myeloid sarcoma, and chloroma tumors which may precede or accompany development of AML (see Ohanian et al., Int J Cancer. 2013 Aug. 1; 133(3): 534-543). EML can occur during or following treatment, and during remission. The incidence of EML in patients with AML of all ages is estimated to be about 9% and EML in children with AML was detected in 40% of patients at diagnosis.

The terms “neoplasia,” “hyperplasia,” and “tumor” are often commonly referred to as “cancer.” which is a general name for more than 100 diseases that are characterized by uncontrolled, abnormal growth of cells.

### Antibodies

As used herein, the term “antigen binding protein” refers to a polypeptide or protein that binds to an antigen, e.g., ILT3 protein. An antigen binding protein includes, but is not limited to, a bivalent antibody tetramer (2H+2L), a monovalent antibody (H+L), a bi-specific antibody that targets an antigen and another target, a Fab fragment, a Fab′ fragment, a F(ab′)2 fragment, an Fv region, and an ScFv. Unless otherwise indicated, the antigen binding proteins herein bind to and inhibit the activity of ILT3.

The term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2^{nd} 15 ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health. Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The term “hypervariable region” refers to the amino acid residues of an antibody that are responsible for antigen-binding. The hypervariable region comprises amino acid residues from a “complementarity determining region” or “CDR” (i.e., CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5^{th} Ed. Public Health Service. National Institutes of Health, Bethesda, Md. (defining the CDR 35 regions of an antibody by sequence); see also Chothia and Lesk (1987) J. Mol. Biol. 196: 901-917 (defining the CDR regions of an antibody by structure). The term “framework” or “FR” residues refers to those variable domain residues other than the hypervariable region residues defined herein as CDR residues.

Unless otherwise indicated, an “antibody fragment” or “antigen binding fragment” refers to antigen binding fragments of antibodies, i.e., antibody fragments that retain the ability to specifically bind to the antigen bound by the full-length antibody, e.g., fragments that retain one or more CDR regions. Examples of antibody binding fragments include, but are not limited to, Fab, Fab′, F(ab′)2, and Fv fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies. or binding fragments thereof, useful in the present invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human ILT3 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“CDR” or “CDRs” means complementarity determining region(s) in an immunoglobulin variable region.

“Framework region” or “FR” as used herein means the immunoglobulin variable regions excluding the CDR regions.

“Isolated antibody” and “isolated antibody fragment” refers to the purification status and in such context means the named molecule is substantially free of other biological molecules such as nucleic acids, proteins, lipids, carbohydrates, or other material such as cellular 10 debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Variable regions” or “V region” as used herein means the segment of IgG chains which is variable in sequence between different antibodies. It extends to Kabat residue 109 in the light chain and 113 in the heavy chain.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The VH and VL regions can be further subdivided into regions of hypervariability, termed complementarity determining regions (CDR), interspersed with regions that are more conserved, termed framework regions (FR). Each VH and VL is composed of three CDR regions and four FR regions, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63: 78-85 (1969), and is shown for the IgG1, IgG2, IgG3, and IgG4 constant domains in Béranger, et al., Ibid.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following:
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al., ILAR Journal 37: 132-141 (1995);
Contact numbering scheme is based on an analysis of the available complex crystal structures (See www.bioinf.org.uk: Prof Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk: Prof Andrew C. R. Martin's Group and reproduced in Table 2 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 2
Antibody CDR Rules

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L51 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat | | | | |
| | Numbering)³ | | | | |
| H1 | H31--H35 | H26--H35 | H26--H32 | H30--H35 | H26--H33 |
| | (Chothia | | | | |
| | Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000).

### Anti-ILT3 Antibodies and Antigen Binding Fragments Useful in the Invention

An “anti-ILT3 antigen binding protein or antigen binding fragment” useful in the any of the treatment methods, compositions and uses of the present invention include monoclonal antibodies (mAb), or antigen binding fragments thereof, which specifically bind to human ILT3. Alternative names or synonyms for ILT3 include: LILRB4; LIR5; and CD85K. In any of the treatment methods, compositions and uses of the present invention in which a human individual is being treated, the anti-ILT3 antigen binding protein, antibody or antigen binding fragment binds to ILT3 and reduces the ability of MDSCs to suppress T-cell activation and proliferation. An anti-ILT3 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

The term “anti-ILT3 antigen binding protein” refers to a protein that binds the extracellular domain (amino acids 22-259) of GenPept Acc. No. Q8NHJ6.3:

(SEQ ID NO: 1)
QAGPLPKPTLWAEPGSVISWGNSVTIWCQGTLEAREYRLDKEESPAPWD
RQNPLEPKNKARFSIPSMTEDYAGRYRCYYRSPVGWSQPSDPLELVMTG
AYSKPTLSALPSPLVTSGKSVTLLCQSRSPMDTFLLIKERAAHPLLHLR
SEHGAQQHQAEFPMSPVTSVHGGTYRCFSSHGFSHYLLSHPSDPLELIV
SGSLEDPRPSPTRSVSTAAGPEDQPLMPTGSVPHSGLRRHWE

Examples of mAbs that bind to human ILT3, useful in the treatment methods and uses of the invention are described in WO2019/099597 (incorporated by reference herein) and summarized below in Table 3.

TABLE 3
Exemplary anti-ILT3 antibody regions and constant regions

| SEQ ID | | |
|---|---|---|
| NO: | Description | Sequence |
| 2 | Human IgG4 HC | ASTKGPSVFPLAPCSRSTSESTAALGCLVKDYFPEPVTVS |
| | Constant domain | WNSGALTSGVHTFPAVLQSSGLYSLSSVVTVPSSSLGTKT |
| | Residue 108 | YTCNVDHKPSNTKVDKRVESKYGPPCPPCPAPEFLGGPSV |
| | corresponds with | FLFPPKPKDTLMISRTPEVTCVVVDVSQEDPEVQFNWYVD |
| | S228P | GVEVHNAKTKPREEQFNSTYRVVSVLTVLHQDWLNGKE |
| | | YKCKVSNKGLPSSIEKTISKAKGQPREPQVYTLPPSQEEMT |
| | | KNQVSLTCLVKGFYPSDIAVEWESNGQPENNYKTTPPVL |
| | | DSDGSFFLYSRLTVDKSRWQEGNVFSCSVMHEALHNHYT |
| | | QKSLSLSLGK |
| 3 | Human IgG4 HC | ASTKGPSVFPLAPCSRSTSESTAALGCLVKDYFPEPVTVS |
| | Constant domain | WNSGALTSGVHTFPAVLQSSGLYSLSSVVTVPSSSLGTKT |
| | Residue 108 | YTCNVDHKPSNTKVDKRVESKYGPPCPPCPAPEFLGGPSV |
| | corresponds with | FLFPPKPKDTLMISRTPEVTCVVVDVSQEDPEVQFNWYVD |
| | S228P | GVEVHNAKTKPREEQFNSTYRVVSVLTVLHQDWLNGKE |
| | (lacks C-terminal | YKCKVSNKGLPSSIEKTISKAKGQPREPQVYTLPPSQEEMT |
| | K, (herein | KNQVSLTCLVKGFYPSDIAVEWESNGQPENNYKTTPPVL |
| | referred to as “K−”) | DSDGSFFLYSRLTVDKSRWQEGNVFSCSVMHEALHNHYT |
| | | QKSLSLSLG |
| 4 | Human IgG1 HC | ASTKGPSVFPLAPSSKSTSGGTAALGCLVKDYFPEPVTVS |
| | constant domain | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSR |
| | | DELTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 5 | Human IgG1 HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | Residue 117 | YICNVNHKPSNTKVDKKVEPKSCDKTHTCPPCPAPEAAG |
| | corresponds with | GPSVFLFPPKPKDTLMISRTPEVTCVVVSVSHEDPEVKEN |
| | L234A, residue | WYVDGVEVHNAKTKPREEQYNSTYRVVSVLTVLHQDWL |
| | 118 corresponds | NGKEYKCKVSNKALPAPIEKTISKAKGQPREPQVYTLPPS |
| | with L235A, | RDELTKNQVSLTCLVKGFYPSDIAVEWESNGQPENNYKT |
| | residue 148 | TPPVLDSDGSFFLYSKLTVDKSRWQQGNVFSCSVMHEAL |
| | corresponds with | HNHYTQKSLSLSPGK |
| | D265S | |
| 6 | Human IgG1 HC | ASTKGPSVFPLAPSSKSTSGGTAALGCLVKDYFPEPVTVS |
| | Constant domain | WNSGALTSGVHTFPAVLQSSGLYSLSSVVTVPSSSLGTQT |
| | (K−) Residue 117 | YICNVNHKPSNTKVDKKVEPKSCDKTHTCPPCPAPEAAG |
| | corresponds with | GPSVFLFPPKPKDTLMISRTPEVTCVVVSVSHEDPEVKFN |
| | L234A, residue | WYVDGVEVHNAKTKPREEQYNSTYRVVSVLTVLHQDWL |
| | 118 corresponds | NGKEYKCKVSNKALPAPIEKTISKAKGQPREPQVYTLPPS |
| | with L235A, | RDELTKNQVSLTCLVKGFYPSDIAVEWESNGQPENNYKT |
| | residue 148 | TPPVLDSDGSFFLYSKLTVDKSRWQQGNVFSCSVMHEAL |
| | corresponds with | HNHYTQKSLSLSPG |
| | D265S | |
| 7 | Human LC | RTVAAPSVFIFPPSDEQLKSGTASVVCLLNNFYPREAKVQ |
| | Kappa Constant | WKVDNALQSGNSQESVTEQDSKDSTYSLSSTLTLSKADY |
| | domain | EKHKVYACEVTHQGLSSPVTKSFNRGEC |
| 8 | Anti-ILT3 52B8 | EVQLVESGGDLVKPGGSLKLSCAASGFTFSNYGMSWVRQ |
| | parental HC | TPDRRLEWVATISGGGDYTNYPDSMRGRFTISRDNAKNT |
| | variable domain | LYLQMSSLKSEDTAMYYCGRRLWFRSLYYAMDYWGQG |
| | | TSVTVSS |
| 9 | Anti-ILT3 52B8 | NIVLTQSPASLAVSLGQRATISCRASEKVDSFGNSFMHWY |
| | parental LC | QQKPGQPPKLLIYLTSNLDSGVPARFSGSGSRTDFALTIDP |
| | variable domain | VEADDAATYYCQQNNEDPYTFGGGTKLEIK |
| 10 | 52B8 HC-CDR1 | NYGMS |
| 11 | 52B8 HC-CDR2 | TISGGGDYTNYPDSXRG |
| | (Wherein Xaa15 | |
| | is M, V, or L) | |
| 12 | 52B8 HC-CDR2 | TISGGGDYTNYPDSMRG |
| | M | |
| 13 | 52B8 HC-CDR2 | TISGGGDYTNYPDSVRG |
| | V | |
| 14 | 52B8 HC-CDR2 | TISGGGDYTNYPDSLRG |
| | L | |
| 15 | 52B8 HC-CDR3 | RLXFRSLYYAMDY |
| | (Wherein Xaa3 is | |
| | W, Y, Q, or F) | |
| 16 | 52B8 HC-CDR3 | RLWFRSLYYAMDY |
| 17 | 52B8 HC-CDR3 | RLYFRSLYYAMDY |
| 18 | 52B8 HC-CDR3 | RLQFRSLYYAMDY |
| 19 | 52B8 HC-CDR3 | RLFFRSLYYAMDY |
| 20 | 52B8 LC-CDR1 | RASEKVDSFGXXFMH |
| | (Wherein Xaa11 | |
| | is N, D, or Q and | |
| | Xaa12 is S, N, or | |
| | A) | |
| 21 | 52B8 LC-CDR1 | RASEKVDSFGNXFMH |
| | N (Wherein | |
| | Xaa12 is S, N, or | |
| | A) | |
| 22 | 52B8 LC-CDR1 | RASEKVDSFGDXFMH |
| | D (Wherein | |
| | Xaa12 is S, N, or | |
| | A) | |
| 23 | 52B8 LC-CDR1 | RASEKVDSFGQXFMH |
| | Q (Wherein | |
| | Xaa12 is S, N, or | |
| | A) | |
| 24 | 52B8 LC-CDR1 | RASEKVDSFGXSFMH |
| | S | |
| | (Wherein Xaa11 | |
| | is N, D, or Q) | |
| 25 | 52B8 LC-CDR1 | RASEKVDSFGXNFMH |
| | N (Wherein | |
| | Xaa11 is N, D, or | |
| | Q) | |
| 26 | 52B8 LC-CDR1 | RASEKVDSFGXAFMH |
| | A (Wherein | |
| | Xaa11 is N, D, or | |
| | Q) | |
| 27 | 52B8 LC-CDR1 | RASEKVDSFGNNFMH |
| | (NN) | |
| 28 | 52B8 LC-CDR1 | RASEKVDSFGDNFMH |
| | (DN) | |
| 29 | 52B8 LC-CDR1 | RASEKVDSFGQNFMH |
| | (QN) | |
| 30 | 52B8 LC-CDR1 | RASEKVDSFGNSFMH |
| | (NS) | |
| 31 | 52B8 LC-CDR1 | RASEKVDSFGDSFMH |
| | (DS) | |
| 32 | 52B8 LC-CDR1 | RASEKVDSFGNAFMH |
| | (NA) | |
| 33 | 52B8 LC-CDR1 | RASEKVDSFGDAFMH |
| | (DA) | |
| 34 | 52B8 LC-CDR1 | RASEKVDSFGQSFMH |
| | (QS) | |
| 35 | 52B8 LC-CDR1 | RASEKVDSFGQAFMH |
| | (AF) | |
| 36 | 52B8 LC-CDR2 | LTSNLDS |
| 37 | 52B8 LC-CDR3 | QQNNEDPYT |
| 38 | Anti-ILT3 40A6 | QVQLKESGPGLVQASETLSLTCTVSGFSLTSYSINWVRQSS |
| | parental HC | GKGPEWMGRFWYDEGIAYNLTLESRLSISGDTSKNQVFL |
| | variable domain | KMNSLRTGDTGTYYCTRDRDTVGITGWFAYWGQGTLVT |
| | | VSS |
| 39 | Anti-ILT3 40A6 | ETVMTQSPTSLSASIGERVTLNCKASQSVGVNVDWYQQT |
| | parental LC | PGQSPKLLIYGSANRHTGVPDRFTGSGFGSDFTLTISDVEP |
| | variable domain | EDLGVYYCLQYGSVPYTFGAGTKLELK |
| 40 | 40A6 HC-CDR1 | SYSIN |
| 41 | 40A6 HC-CDR2 | RFWYDEGIAYNLTLES |
| 42 | 40A6 HC-CDR3 | DRDTVGITGWFAY |
| 43 | 40A6 LC-CDR1 | KASQSVGVNVD |
| 44 | 40A6 LC-CDR2 | GSANRHT |
| 45 | 40A6 LC-CDR3 | LQYGSVPYT |
| 46 | Anti-ILT3 16B1 | QVQLKESGPGLVQASETLSLTCTVSGFSLTNYCVNWVRQ |
| | parental HC | PSGKGPEWLGRFWFDEGKAYNLTLESRLSISGDTSKNQVF |
| | variable domain | LRMNSLRADDTGTYYCTRDRDTVGITGWFAYWGQGTLV |
| | | TVSS |
| 47 | Anti-ILT3 16B1 | ETVMTQSPTSLSASIGERVTLNCKASQSVGINVDWYQQTP |
| | parental LC | GQSPKLLIYGSANRHTGVPDRFTGSGFGSDFTLTISNVEPE |
| | variable domain | DLGVYYCLQYGSVPYTFGPGTKLELK |
| 48 | 16B1 HC-CDR1 | NYCVN |
| 49 | 16B1 HC-CDR2 | RFWFDEGKAYNLTLES |
| 50 | 16B1 HC-CDR3 | DRDTVGITGWFAY |
| 51 | 16B1 LC-CDR1 | KASQSVGINVD |
| 52 | 16B1 LC-CDR2 | GSANRHT |
| 53 | 16B1 LC-CDR3 | LQYGSVPYT |
| 54 | Anti-ILT3 11D1 | QVQLQQSGAELMKPGASVKISCKATGYTFRTYWIEWVKQ |
| | parental HC | RPGHGLEWIGEILPGNGNTHENENFKDKATFTADTSSNAA |
| | variable domain | YMQLSSLTSEDSAVYYCVRRLGRGPFDFWGQGTTLTVSS |
| 55 | Anti-ILT3 11D1 | DIQMTQSPSSLSVSLGGKVTITCKASQDINEYIGWYQRKP |
| | parental LC | GKGPRLLIHYTSTLQSGIPSRFSGSGSGRDYSLSISNLEPEDI |
| | variable domain | ATYYCLQYANPLPTFGGGTKLEIK |
| 56 | 11D1 HC-CDR1 | TYWIE |
| 57 | 11D1 HC-CDR2 | EILPGNGNTHENENFKD |
| 58 | 11DI HC-CDR3 | RRLGRGPFDF |
| 59 | 11D1 LC-CDR1 | KASQDINEYIG |
| 60 | 11D1 LC-CDR2 | YTSTLQS |
| 61 | 11D1 LC-CDR3 | LQYANPLPT |
| 62 | Anti-ILT3 | EVQLVESGGGLVQPGRSMKLSCAASGFTFSNFDMAWVR |
| | 17H12 parental | QAPTRGLEWVSSITYDGGSTSYRDSVKGRFTISRDNAKGT |
| | HC variable | LYLQMDSLRSEDTATYYCTTVESIATISTYFDYWGQGVM |
| | domain | VTVSS |
| 63 | Anti-ILT3 | DIVLTQSPALAVSLGQRATISCRASQSVSMSRYDLIHWYQ |
| | 17H12 parental | QKPGQQPKLLIFRASDLASGIPARFSGSGSGTDFTLTINPVQ |
| | LC variable | ADDIATYYCQQTRKSPPTFGGGTRLELK |
| | domain | |
| 64 | 17H12 HC- | NFDMA |
| | CDR1 | |
| 65 | 17H12 HC- | SITYDGGSTSYRDSVKG |
| | CDR2 | |
| 66 | 17H12 HC- | VESIATISTYFDY |
| | CDR3 | |
| 67 | 17H12 LC- | RASQSVSMSRYDLIH |
| | CDR1 | |
| 68 | 17H12 LC- | RASDLAS |
| | CDR2 | |
| 69 | 17H12 LC- | QQTRKSPPT |
| | CDR3 | |
| 70 | Anti-ILT3 37C8 | QVQLKESGPGLVQASETLSLTCTVSGFSLTSYCVNWVRQP |
| | parental HC | SGKGPEWLGRFWYDEGKVYNLTLESRLSISGDTSKNQVF |
| | variable domain | LKMNRLRTDDTGTYYCTRDRDTMGITGWFAYWGQGTLV |
| | | TVSS |
| 71 | Anti-ILT3 37C8 | ETVMTQSPTSLSASIGERVTLNCKASQSVGINVDWYQQTP |
| | parental LC | GQSPKLLIYGSANRHTGVPDRFTGSGFGSGFTLTISNVEPE |
| | variable domain | DLGVYYCLQYGSVPYTFGPGTKLELK |
| 72 | 37C8 HC-CDR1 | SYCVN |
| 73 | 37C8 HC-CDR2 | RFWYDEGKVYNLTLES |
| 74 | 37C8 HC-CDR3 | DRDTMGITGWFAY |
| 75 | 37C8 LC-CDR1 | KASQSVGINVD |
| 76 | 37C8 LC-CDR2 | GSANRHT |
| 77 | 37C8 LC-CDR3 | LQYGSVPYT |
| 78 | Anti-ILT3 1G12 | QVQMQQSGTELMKPGASMKISCKATGYTFSTYWIQWIKQ |
| | parental HC | RPGHGLEWIGEILPGSGTTNYNENFKGKATFSADTSSNTA |
| | variable domain | YIHLSSLTSEDSAVFYCARRLGRGPFDYWGQGTTLTVSS |
| 79 | Anti-ILT3 1G12 | DIQMTQSPSSLSASLGGKVTITCEASQDINKHIDWYQHQP |
| | parental LC | GRGPSLLIHYASILQPGIPSRFSGSGSGRDYSFSITSLEPEDI |
| | variable domain | ATYYCLQYDNLLPTFGGGTKLEIK |
| 80 | 1G12 HC-CDR1 | TYWIQ |
| 81 | 1G12 HC-CDR2 | EILPGSGTTNYNENFKG |
| 82 | 1G12 HC-CDR3 | RLGRGPFDY |
| 83 | 1G12 LC-CDR1 | EASQDINKHID |
| 84 | 1G12 LC-CDR2 | YASILQP |
| 85 | 1G12 LC-CDR3 | LQYDNLLPT |
| 86 | Anti-ILT3 20E4 | QVQLKESGPGLVQASETLSLTCTVSGFSLTSYSVNWVRQP |
| | parental HC | SGKGLEWMGRFWYDGGTAYNSTLESRLSISGDTSKNQVF |
| | variable domain | LKMNSLQTDDTGTYYCTRDRDTMGITGWFAYWGQGTLV |
| | | TVSP |
| 87 | Anti-ILT3 20E4 | ETVMTQSPTSLSASIGERVTLNCKASQSVGVNVDWYQQT |
| | parental LC | PGQSPKLLIYGSANRHTGVPDRFTGSGFGSDFTLTISNVEP |
| | variable domain | EDLGVYYCLQYGSVPYTFGAGTKLELK |
| 88 | 20E4 HC-CDR1 | SYSVN |
| 89 | 20E4 HC-CDR2 | RFWYDGGTAYNSTLES |
| 90 | 20E4 HC-CDR3 | DRDTMGITGWFAY |
| 91 | 20E4 LC-CDR1 | KASQSVGVNVD |
| 92 | 20E4 LC-CDR2 | GSANRHT |
| 93 | 20E4 LC-CDR3 | LQYGSVPYT |
| 94 | Anti-ILT3 24A4 | QVQLKESGPGLVQASETLSLTCTVSGFSLTSYCVNWVRQP |
| | parental HC | SGKGPEWLGRFWYDEGKVYNLTLESRLSISGDTSKNQVF |
| | variable domain | LKMNRLRTDDTGTYYCTRDRDTLGITGWFAYWGQGTLV |
| | | TVSS |
| 95 | Anti-ILT3 24A4 | ETVMTQSPTSLSASIGERVTLNCKASQSVGINVDWYQQTP |
| | parental LC | GQSPKLLIYGSANRHTGVPDRFTGSGFGSGFTLTISNVEPE |
| | variable domain | DLGVYYCLQYGSVPYTFGPGTKLELK |
| 96 | 24A4 HC-CDR1 | SYCVN |
| 97 | 24A4 HC-CDR2 | RFWYDEGKVYNLTLES |
| 98 | 24A4 HC-CDR3 | DRDTLGITGWFAY |
| 99 | 24A4 LC-CDR1 | KASQSVGINVD |
| 100 | 24A4 LC-CDR2 | GSANRHT |
| 101 | 24A4 LC-CDR3 | LQYGSVPYT |
| 102 | Leader sequence | MEWSWVFLFFLSVTTGVHS |
| | A | |
| 103 | Leader sequence | MSVPTQVLGLLLLWLTDARC |
| | B | |
| 104 | Mouse Anti- | EVQLVESGGDLVKPGGSLKLSCAASGFTFSNYGMSWVRQ |
| | ILT3 p52B8 | TPDRRLEWVATISGGGDYTNYPDSMRGRFTISRDNAKNT |
| | parental HC: | LYLQMSSLKSEDTAMYYCGRRLWFRSLYYAMDYWGQG |
| | Murine IgG2a | TSVTVSSAKTTAPSVYPLAPVCGDTTGSSVTLGCLVKGYF |
| | heavy chain | PEPVTLTWNSGSLSSGVHTFPAVLQSDLYTLSSSVTVTSST |
| | | WPSQSITCNVAHPASSTKVDKKIEPRGPTIKPCPPCKCPAP |
| | | NLLGGPSVFIFPPKIKDVLMISLSPIVTCVVVDVSEDDPDV |
| | | QISWFVNNVEVHTAQTQTHREDYNSTLRVVSALPIQHQD |
| | | WMSGKEFKCKVNNKDLPAPIERTISKPKGSVRAPQVYVLP |
| | | PPEEEMTKKQVTLTCMVTDFMPEDIYVEWTNNGKTELNY |
| | | KNTEPVLDSDGSYFMYSKLRVEKKNWVERNSYSCSVVHE |
| | | GLHNHHTTKSFSRTPGK |
| 105 | Mouse Anti- | NIVLTQSPASLAVSLGQRATISCRASEKVDSFGNSFMHWY |
| | ILT3 p52B8 | QQKPGQPPKLLIYLTSNLDSGVPARFSGSGSRTDFALTIDP |
| | parental LC: | VEADDAATYYCQQNNEDPYTFGGGTKLEIKRADAAPTVS |
| | murine Kappa | IFPPSSEQLTSGGASVVCFLNNFYPKDINVKWKIDGSERQN |
| | light chain | GVLNSWTDQDSKDSTYSMSSTLTLTKDEYERHNSYTCEA |
| | | THKTSTSPIVKSFNRNEC |
| 106 | Chimeric Anti- | EVQLVESGGDLVKPGGSLKLSCAASGFTFSNYGMSWVRQ |
| | ILT3 mouse | TPDRRLEWVATISGGGDYTNYPDSMRGRFTISRDNAKNT |
| | 52B8 VH | LYLQMSSLKSEDTAMYYCGRRLWFRSLYYAMDYWGQG |
| | parental/human | TSVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | | SLGTKTYTCNVDHKPSNTKVDKRVESKYGPPCPPCPAPEF |
| | | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 107 | Chimeric Anti- | EVQLVESGGDLVKPGGSLKLSCAASGFTFSNYGMSWVRQ |
| | ILT3 mouse | TPDRRLEWVATISGGGDYTNYPDSVRGRFTISRDNAKNTL |
| | 52B8 VH | YLQMSSLKSEDTAMYYCGRRLWFRSLYYAMDYWGQGT |
| | M64V/human | SVTVSSASTKGPSVFPLAPCSRSTSESTAALGCLVKDYFPE |
| | IgG4 (S228P) | PVTVSWNSGALTSGVHTFPAVLQSSGLYSLSSVVTVPSSS |
| | | LGTKTYTCNVDHKPSNTKVDKRVESKYGPPCPPCPAPEFL |
| | | GGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 108 | Mouse Anti- | EVQLVESGGDLVKPGGSLKLSCAASGFTFSNYGMSWVRQ |
| | ILT3 52B8 VH | TPDRRLEWVATISGGGDYTNYPDSLRGRFTISRDNAKNTL |
| | M64L/human | YLQMSSLKSEDTAMYYCGRRLWFRSLYYAMDYWGQGT |
| | IgG4 (S228P) | SVTVSSASTKGPSVFPLAPCSRSTSESTAALGCLVKDYFPE |
| | | PVTVSWNSGALTSGVHTFPAVLQSSGLYSLSSVVTVPSSS |
| | | LGTKTYTCNVDHKPSNTKVDKRVESKYGPPCPPCPAPEFL |
| | | GGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 109 | Chimeric Anti- | NIVLTQSPASLAVSLGQRATISCRASEKVDSFGNSFMHWY |
| | ILT3 mouse | QQKPGQPPKLLIYLTSNLDSGVPARFSGSGSRTDFALTIDP |
| | 52B8 parental | VEADDAATYYCQQNNEDPYTFGGGTKLEIKRTVAAPSVFI |
| | VL/human | FPPSDEQLKSGTASVVCLLNNFYPREAKVQWKVDNALQS |
| | Kappa | GNSQESVTEQDSKDSTYSLSSTLTLSKADYEKHKVYACEV |
| | | THQGLSSPVTKSFNRGEC |
| 110 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain VH1 | LYLQMNSLRAEDTAVYYCGRRLWFRSLYYAMDYWGQG |
| | | TLVTVSS |
| 111 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLWFRSLYYAMDYWGQG |
| | (M64V) | TLVTVSS |
| 112 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH1 | YLQMNSLRAEDTAVYYCGRRLWFRSLYYAMDYWGQGT |
| | (M64L) | LVTVSS |
| 113 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLFFRSLYYAMDYWGQGT |
| | (M64V, W101F) | LVTVSS |
| 114 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLYFRSLYYAMDYWGQG |
| | (M64V, W101Y) | TLVTVSS |
| 115 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLQFRSLYYAMDYWGQG |
| | (M64V, W101Q) | TLVTVSS |
| 116 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain VH2 | LYLQMNSLKAEDTAVYYCGRRLWFRSLYYAMDYWGQG |
| | | TLVTVSS |
| 117 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH2 | LYLQMNSLKAEDTAVYYCGRRLWFRSLYYAMDYWGQG |
| | (M64V) | TLVTVSS |
| 118 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH2 | YLQMNSLKAEDTAVYYCGRRLWFRSLYYAMDYWGQGT |
| | (M64L) | LVTVSS |
| 119 | Humanized 52B8 | DIVLTQSPDSLAVSLGERATINCRASEKVDSFGNSFMHWY |
| | LC variable | QQKPGQPPKLLIYLTSNLDSGVPDRFSGSGSRTDFTLTISSL |
| | domain VL1 | QAEDVAVYYCQQNNEDPYTFGQGTKLEIK |
| 120 | Humanized 52B8 | DIVLTQSPDSLAVSLGERATINCRASEKVDSFGNSFMHWY |
| | LC variable | QQKPGQPPKLLIYLTSNLDSGVPDRFSGSGSGTDFTLTISSL |
| | domain VL2 | QAEDVAVYYCQQNNEDPYTFGQGTKLEIK |
| 121 | Humanized 52B8 | EIVLTQSPATLSLSPGERATLSCRASEKVDSFGNSFMHWY |
| | LC variable | QQKPGQAPRLLIYLTSNLDSGVPARFSGSGSRTDFTLTISSL |
| | domain VL3 | EPEDFAVYYCQQNNEDPYTFGQGTKLEIK |
| 122 | Humanized 52B8 | EIVLTQSPATLSLSPGERATLSCRASEKVDSFGNSFMHWY |
| | LC variable | QQKPGQAPRLLIYLTSNLDSGIPARFSGSGSGTDFTLTISSL |
| | domain VL4 | EPEDFAVYYCQQNNEDPYTFGQGTKLEIK |
| 123 | Humanized 52B8 | DIQLTQSPSSLSASVGDRVTITCRASEKVDSFGNSFMHWY |
| | LC variable | QQKPGKAPKLLIYLTSNLDSGVPSRFSGSGSGTDFTLTISSL |
| | domain VL5 | QPEDFATYYCQQNNEDPYTFGQGTKLEIK |
| 124 | Humanized 52B8 | DIQMTQSPSSLSASVGDRVTITCRASEKVDSFGNSFMHWY |
| | LC variable | QQKPGKAPKLLIYLTSNLDSGVPSRFSGSGSRTDFTLTISSL |
| | domain VL6 | QPEDFATYYCQQNNEDPYTFGQGTKLEIK |
| 125 | Humanized 52B8 | DIQLTQSPSSLSASVGDRVTITCRASEKVDSFGNSFMHWY |
| | LC variable | QQKPGKAPKLLIYLTSNLDSGVPSRFSGSGSRTDFTLTISSL |
| | domain VL7 | QPEDFATYYCQQNNEDPYTFGQGTKLEIK |
| 126 | Humanized 52B8 | DIQLTQSPSSLSASVGDRVTITCRASEKVDSFGNSFMHWY |
| | LC variable | QQKPGKAPKLLIYLTSNLDSGVPARFSGSGSRTDFTLTISS |
| | domain VL8 | LQPEDFATYYCQQNNEDPYTFGQGTKLEIK |
| 127 | Humanized 52B8 | DIVLTQSPDSLAVSLGERATINCRASEKVDSFGNAFMHWY |
| | LC variable | QQKPGQPPKLLIYLTSNLDSGVPDRFSGSGSGTDFTLTISSL |
| | domain VL2, | QAEDVAVYYCQQNNEDPYTFGQGTKLEIK |
| | (S35A) | |
| 128 | Humanized 52B8 | DIVLTQSPDSLAVSLGERATINCRASEKVDSFGNNFMHWY |
| | LC variable | QQKPGQPPKLLIYLTSNLDSGVPDRFSGSGSGTDFTLTISSL |
| | domain VL2, | QAEDVAVYYCQQNNEDPYTFGQGTKLEIK |
| | (S35N) | |
| 129 | Humanized 52B8 | DIVLTQSPDSLAVSLGERATINCRASEKVDSFGQSFMHWY |
| | LC variable | QQKPGQPPKLLIYLTSNLDSGVPDRFSGSGSGTDFTLTISSL |
| | domain VL2, | QAEDVAVYYCQQNNEDPYTFGQGTKLEIK |
| | (N34Q) | |
| 130 | Humanized 52B8 | DIVLTQSPDSLAVSLGERATINCRASEKVDSFGDSFMHWY |
| | LC variable | QQKPGQPPKLLIYLTSNLDSGVPDRFSGSGSGTDFTLTISSL |
| | domain VL2, | QAEDVAVYYCQQNNEDPYTFGQGTKLEIK |
| | (N34D) | |
| 131 | Humanized 52B8 | DIQLTQSPSSLSASVGDRVTITCRASEKVDSFGNAFMHWY |
| | LC variable | QQKPGKAPKLLIYLTSNLDSGVPSRFSGSGSGTDFTLTISSL |
| | domain VL5, | QPEDFATYYCQQNNEDPYTFGQGTKLEIK |
| | (S35A) | |
| 132 | Humanized 52B8 | DIQLTQSPSSLSASVGDRVTITCRASEKVDSFGNNFMHWY |
| | LC variable | QQKPGKAPKLLIYLTSNLDSGVPSRFSGSGSGTDFTLTISSL |
| | domain VL5, | QPEDFATYYCQQNNEDPYTFGQGTKLEIK |
| | (S35N) | |
| 133 | Humanized 52B8 | DIQLTQSPSSLSASVGDRVTITCRASEKVDSFGQSFMHWY |
| | LC variable | QQKPGKAPKLLIYLTSNLDSGVPSRFSGSGSGTDFTLTISSL |
| | domain VL5 | QPEDFATYYCQQNNEDPYTFGQGTKLEIK |
| | (N34Q) | |
| 134 | Humanized 52B8 | DIQLTQSPSSLSASVGDRVTITCRASEKVDSFGDSFMHWY |
| | LC variable | QQKPGKAPKLLIYLTSNLDSGVPSRFSGSGSGTDFTLTISSL |
| | domain VL5, | QPEDFATYYCQQNNEDPYTFGQGTKLEIK |
| | (N34D) | |
| 135 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain | LYLQMNSLRAEDTAVYYCGRRLWFRSLYYAMDYWGQG |
| | VH1/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | constant domain | SLGTKTYTCNVDHKPSNTKVDKRVESKYGPPCPPCPAPEF |
| | | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 136 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLWFRSLYYAMDYWGQG |
| | (M64V)/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | constant domain | SLGTKTYTCNVDHKPSNTKVDKRVESKYGPPCPPCPAPEF |
| | | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 137 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH1 | YLQMNSLRAEDTAVYYCGRRLWFRSLYYAMDYWGQGT |
| | (M64L)/Human | LVTVSSASTKGPSVFPLAPCSRSTSESTAALGCLVKDYFPE |
| | IgG4 (S228P) | PVTVSWNSGALTSGVHTFPAVLQSSGLYSLSSVVTVPSSS |
| | constant domain | LGTKTYTCNVDHKPSNTKVDKRVESKYGPPCPPCPAPEFL |
| | | GGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 138 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLFFRSLYYAMDYWGQGT |
| | (M64V, | LVTVSSASTKGPSVFPLAPCSRSTSESTAALGCLVKDYFPE |
| | W101F)/Human | PVTVSWNSGALTSGVHTFPAVLQSSGLYSLSSVVTVPSSS |
| | IgG4 (S228P) | LGTKTYTCNVDHKPSNTKVDKRVESKYGPPCPPCPAPEFL |
| | constant domain | GGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 139 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLYFRSLYYAMDYWGQG |
| | (M64V, | TLVTVSSASTKGPSVFPLAPCSRSTSESTAALGCLVKDYFP |
| | W101Y)/Human | EPVTVSWNSGALTSGVHTFPAVLQSSGLYSLSSVVTVPSS |
| | IgG4 (S228P) | SLGTKTYTCNVDHKPSNTKVDKRVESKYGPPCPPCPAPEF |
| | constant domain | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 140 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLQFRSLYYAMDYWGQG |
| | (M64V, | TLVTVSSASTKGPSVFPLAPCSRSTSESTAALGCLVKDYFP |
| | W101Q)/Human | EPVTVSWNSGALTSGVHTFPAVLQSSGLYSLSSVVTVPSS |
| | IgG4 (S228P) | SLGTKTYTCNVDHKPSNTKVDKRVESKYGPPCPPCPAPEF |
| | constant domain | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 141 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain | LYLQMNSLKAEDTAVYYCGRRLWFRSLYYAMDYWGQG |
| | VH2/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | constant domain | SLGTKTYTCNVDHKPSNTKVDKRVESKYGPPCPPCPAPEF |
| | | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 142 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH2 | LYLQMNSLKAEDTAVYYCGRRLWFRSLYYAMDYWGQG |
| | (M64V)/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | constant domain | SLGTKTYTCNVDHKPSNTKVDKRVESKYGPPCPPCPAPEF |
| | | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 143 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH2 | YLQMNSLKAEDTAVYYCGRRLWFRSLYYAMDYWGQGT |
| | (M64L)/Human | LVTVSSASTKGPSVFPLAPCSRSTSESTAALGCLVKDYFPE |
| | IgG4 (S228P) | PVTVSWNSGALTSGVHTFPAVLQSSGLYSLSSVVTVPSSS |
| | constant domain | LGTKTYTCNVDHKPSNTKVDKRVESKYGPPCPPCPAPEFL |
| | | GGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLGK |
| 144 | Humanized 52B8 | DIVLTQSPDSLAVSLGERATINCRASEKVDSFGNSFMHWY |
| | LC variable | QQKPGQPPKLLIYLTSNLDSGVPDRFSGSGSRTDFTLTISSL |
| | domain | QAEDVAVYYCQQNNEDPYTFGQGTKLEIKRTVAAPSVFIF |
| | VL1/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 145 | Humanized 52B8 | DIVLTQSPDSLAVSLGERATINCRASEKVDSFGNSFMHWY |
| | LC variable | QQKPGQPPKLLIYLTSNLDSGVPDRFSGSGSGTDFTLTISSL |
| | domain | QAEDVAVYYCQQNNEDPYTFGQGTKLEIKRTVAAPSVFIF |
| | VL2/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 146 | Humanized 52B8 | EIVLTQSPATLSLSPGERATLSCRASEKVDSFGNSFMHWY |
| | LC variable | QQKPGQAPRLLIYLTSNLDSGVPARFSGSGSRTDFTLTISSL |
| | domain | EPEDFAVYYCQQNNEDPYTFGQGTKLEIKRTVAAPSVFIF |
| | VL3/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 147 | Humanized 52B8 | EIVLTQSPATLSLSPGERATLSCRASEKVDSFGNSFMHWY |
| | LC variable | QQKPGQAPRLLIYLTSNLDSGIPARFSGSGSGTDFTLTISSL |
| | domain | EPEDFAVYYCQQNNEDPYTFGQGTKLEIKRTVAAPSVFIF |
| | VL4/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 148 | Humanized 52B8 | DIQLTQSPSSLSASVGDRVTITCRASEKVDSFGNSFMHWY |
| | LC variable | QQKPGKAPKLLIYLTSNLDSGVPSRFSGSGSGTDFTLTISSL |
| | domain | QPEDFATYYCQQNNEDPYTFGQGTKLEIKRTVAAPSVFIF |
| | VL5/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 149 | Humanized 52B8 | DIQMTQSPSSLSASVGDRVTITCRASEKVDSFGNSFMHWY |
| | LC variable | QQKPGKAPKLLIYLTSNLDSGVPSRFSGSGSRTDFTLTISSL |
| | domain | QPEDFATYYCQQNNEDPYTFGQGTKLEIKRTVAAPSVFIF |
| | VL6/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 150 | Humanized 52B8 | DIQLTQSPSSLSASVGDRVTITCRASEKVDSFGNSFMHWY |
| | LC variable | QQKPGKAPKLLIYLTSNLDSGVPSRFSGSGSRTDFTLTISSL |
| | domain | QPEDFATYYCQQNNEDPYTFGQGTKLEIKRTVAAPSVFIF |
| | VL7/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 151 | Humanized 52B8 | DIQLTQSPSSLSASVGDRVTITCRASEKVDSFGNSFMHWY |
| | LC variable | QQKPGKAPKLLIYLTSNLDSGVPARFSGSGSRTDFTLTISS |
| | domain | LQPEDFATYYCQQNNEDPYTFGQGTKLEIKRTVAAPSVFI |
| | VL8/kappa | FPPSDEQLKSGTASVVCLLNNFYPREAKVQWKVDNALQS |
| | constant domain | GNSQESVTEQDSKDSTYSLSSTLTLSKADYEKHKVYACEV |
| | | THQGLSSPVTKSFNRGEC |
| 152 | Humanized 52B8 | DIVLTQSPDSLAVSLGERATINCRASEKVDSFGNAFMHWY |
| | LC variable | QQKPGQPPKLLIYLTSNLDSGVPDRFSGSGSGTDFTLTISSL |
| | domain VL2 | QAEDVAVYYCQQNNEDPYTFGQGTKLEIKRTVAAPSVFIF |
| | (S35A)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 153 | Humanized 52B8 | DIVLTQSPDSLAVSLGERATINCRASEKVDSFGNNFMHWY |
| | LC variable | QQKPGQPPKLLIYLTSNLDSGVPDRFSGSGSGTDFTLTISSL |
| | domain VL2 | QAEDVAVYYCQQNNEDPYTFGQGTKLEIKRTVAAPSVFIF |
| | (S35N)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 154 | Humanized 52B8 | DIVLTQSPDSLAVSLGERATINCRASEKVDSFGQSFMHWY |
| | LC variable | QQKPGQPPKLLIYLTSNLDSGVPDRFSGSGSGTDFTLTISSL |
| | domain VL2 | QAEDVAVYYCQQNNEDPYTFGQGTKLEIKRTVAAPSVFIF |
| | (N34Q)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 155 | Humanized 52B8 | DIVLTQSPDSLAVSLGERATINCRASEKVDSFGDSFMHWY |
| | LC variable | QQKPGQPPKLLIYLTSNLDSGVPDRFSGSGSGTDFTLTISSL |
| | domain VL2 | QAEDVAVYYCQQNNEDPYTFGQGTKLEIKRTVAAPSVFIF |
| | (N34D)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 156 | Humanized 52B8 | DIQLTQSPSSLSASVGDRVTITCRASEKVDSFGNAFMHWY |
| | LC variable | QQKPGKAPKLLIYLTSNLDSGVPSRFSGSGSGTDFTLTISSL |
| | domain VL5 | QPEDFATYYCQQNNEDPYTFGQGTKLEIKRTVAAPSVFIF |
| | (S35A)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 157 | Humanized 52B8 | DIQLTQSPSSLSASVGDRVTITCRASEKVDSFGNNFMHWY |
| | LC variable | QQKPGKAPKLLIYLTSNLDSGVPSRFSGSGSGTDFTLTISSL |
| | domain VL5 | QPEDFATYYCQQNNEDPYTFGQGTKLEIKRTVAAPSVFIF |
| | (S35N)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 158 | Humanized 52B8 | DIQLTQSPSSLSASVGDRVTITCRASEKVDSFGQSFMHWY |
| | LC variable | QQKPGKAPKLLIYLTSNLDSGVPSRFSGSGSGTDFTLTISSL |
| | domain VL5 | QPEDFATYYCQQNNEDPYTFGQGTKLEIKRTVAAPSVFIF |
| | (N34Q)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 159 | Humanized 52B8 | DIQLTQSPSSLSASVGDRVTITCRASEKVDSFGDSFMHWY |
| | LC variable | QQKPGKAPKLLIYLTSNLDSGVPSRFSGSGSGTDFTLTISSL |
| | domain VL5 | QPEDFATYYCQQNNEDPYTFGQGTKLEIKRTVAAPSVFIF |
| | (N34D)/kappa | PPSDEQLKSGTASVVCLLNNFYPREAKVQWKVDNALQSG |
| | constant domain | NSQESVTEQDSKDSTYSLSSTLTLSKADYEKHKVYACEVT |
| | | HQGLSSPVTKSFNRGEC |
| 160 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain VH1/ | LYLQMNSLRAEDTAVYYCGRRLWFRSLYYAMDYWGQG |
| | Human IgG1 HC | TLVTVSSASTKGPSVFPLAPSSKSTSGGTAALGCLVKDYFP |
| | (L234A L235A | EPVTVSWNSGALTSGVHTFPAVLQSSGLYSLSSVVTVPSS |
| | D265S) constant | SLGTQTYICNVNHKPSNTKVDKKVEPKSCDKTHTCPPCPA |
| | domain | PEAAGGPSVFLFPPKPKDTLMISRTPEVTCVVVSVSHEDPE |
| | | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 161 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLWFRSLYYAMDYWGQG |
| | (M64V)/Human | TLVTVSSASTKGPSVFPLAPSSKSTSGGTAALGCLVKDYFP |
| | IgG1 HC | EPVTVSWNSGALTSGVHTFPAVLQSSGLYSLSSVVTVPSS |
| | (L234A, L235A, | SLGTQTYICNVNHKPSNTKVDKKVEPKSCDKTHTCPPCPA |
| | D265S) constant | PEAAGGPSVFLFPPKPKDTLMISRTPEVTCVVVSVSHEDPE |
| | domain | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 162 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH1 | YLQMNSLRAEDTAVYYCGRRLWFRSLYYAMDYWGQGT |
| | (M64L)/Human | LVTVSSASTKGPSVFPLAPSSKSTSGGTAALGCLVKDYFPE |
| | IgG1 HC | PVTVSWNSGALTSGVHTFPAVLQSSGLYSLSSVVTVPSSS |
| | (L234A, L235A, | LGTQTYICNVNHKPSNTKVDKKVEPKSCDKTHTCPPCPAP |
| | D265S) constant | EAAGGPSVFLFPPKPKDTLMISRTPEVTCVVVSVSHEDPEV |
| | domain | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 163 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLFFRSLYYAMDYWGQGT |
| | (M64V, W101F)/ | LVTVSSASTKGPSVFPLAPSSKSTSGGTAALGCLVKDYFPE |
| | Human IgG1 HC | PVTVSWNSGALTSGVHTFPAVLQSSGLYSLSSVVTVPSSS |
| | (L234A, L235A, | LGTQTYICNVNHKPSNTKVDKKVEPKSCDKTHTCPPCPAP |
| | D265S) constant | EAAGGPSVFLFPPKPKDTLMISRTPEVTCVVVSVSHEDPEV |
| | domain | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 164 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLYFRSLYYAMDYWGQG |
| | (M64V, | TLVTVSSASTKGPSVFPLAPSSKSTSGGTAALGCLVKDYFP |
| | W101Y)/Human | EPVTVSWNSGALTSGVHTFPAVLQSSGLYSLSSVVTVPSS |
| | IgG1 HC | SLGTQTYICNVNHKPSNTKVDKKVEPKSCDKTHTCPPCPA |
| | (L234A, L235A, | PEAAGGPSVFLFPPKPKDTLMISRTPEVTCVVVSVSHEDPE |
| | D265S) constant | VKFNWYVDGVEVHNAKTKPREEQYNSTYRVVSVLTVLH |
| | domain | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 165 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLQFRSLYYAMDYWGQG |
| | (M64V, | TLVTVSSASTKGPSVFPLAPSSKSTSGGTAALGCLVKDYFP |
| | W101Q)/Human | EPVTVSWNSGALTSGVHTFPAVLQSSGLYSLSSVVTVPSS |
| | IgG1 HC | SLGTQTYICNVNHKPSNTKVDKKVEPKSCDKTHTCPPCPA |
| | (L234A, L235A, | PEAAGGPSVFLFPPKPKDTLMISRTPEVTCVVVSVSHEDPE |
| | D265S) constant | VKFNWYVDGVEVHNAKTKPREEQYNSTYRVVSVLTVLH |
| | domain | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 166 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain VH2/ | LYLQMNSLKAEDTAVYYCGRRLWFRSLYYAMDYWGQG |
| | Human IgG1 HC | TLVTVSSASTKGPSVFPLAPSSKSTSGGTAALGCLVKDYFP |
| | (L234A, L235A, | EPVTVSWNSGALTSGVHTFPAVLQSSGLYSLSSVVTVPSS |
| | D265S) constant | SLGTQTYICNVNHKPSNTKVDKKVEPKSCDKTHTCPPCPA |
| | domain | PEAAGGPSVFLFPPKPKDTLMISRTPEVTCVVVSVSHEDPE |
| | | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 167 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH2 | LYLQMNSLKAEDTAVYYCGRRLWFRSLYYAMDYWGQG |
| | (M64V)/Human | TLVTVSSASTKGPSVFPLAPSSKSTSGGTAALGCLVKDYFP |
| | IgG1 HC | EPVTVSWNSGALTSGVHTFPAVLQSSGLYSLSSVVTVPSS |
| | (L234A, L235A, | SLGTQTYICNVNHKPSNTKVDKKVEPKSCDKTHTCPPCPA |
| | D265S) constant | PEAAGGPSVFLFPPKPKDTLMISRTPEVTCVVVSVSHEDPE |
| | domain | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 168 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH2 | YLQMNSLKAEDTAVYYCGRRLWFRSLYYAMDYWGQGT |
| | (M64L)/Human | LVTVSSASTKGPSVFPLAPSSKSTSGGTAALGCLVKDYFPE |
| | IgG1 HC | PVTVSWNSGALTSGVHTFPAVLQSSGLYSLSSVVTVPSSS |
| | (L234A, L235A, | LGTQTYICNVNHKPSNTKVDKKVEPKSCDKTHTCPPCPAP |
| | D265S) constant | EAAGGPSVFLFPPKPKDTLMISRTPEVTCVVVSVSHEDPEV |
| | domain | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 169 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain | LYLQMNSLRAEDTAVYYCGRRLWFRSLYYAMDYWGQG |
| | VH1/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | (K−) constant | SLGTKTYTCNVDHKPSNTKVDKRVESKYGPPCPPCPAPEF |
| | domain | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 170 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLWFRSLYYAMDYWGQG |
| | (M64V)/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | (K−) constant | SLGTKTYTCNVDHKPSNTKVDKRVESKYGPPCPPCPAPEF |
| | domain | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 171 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH1 | YLQMNSLRAEDTAVYYCGRRLWFRSLYYAMDYWGQGT |
| | (M64L)/Human | LVTVSSASTKGPSVFPLAPCSRSTSESTAALGCLVKDYFPE |
| | IgG4 (S228P) | PVTVSWNSGALTSGVHTFPAVLQSSGLYSLSSVVTVPSSS |
| | (K−) constant | LGTKTYTCNVDHKPSNTKVDKRVESKYGPPCPPCPAPEFL |
| | domain | GGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 172 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLFFRSLYYAMDYWGQGT |
| | (M64V), | LVTVSSASTKGPSVFPLAPCSRSTSESTAALGCLVKDYFPE |
| | W101F/Human | PVTVSWNSGALTSGVHTFPAVLQSSGLYSLSSVVTVPSSS |
| | IgG4 (S228P) | LGTKTYTCNVDHKPSNTKVDKRVESKYGPPCPPCPAPEFL |
| | (K−) constant | GGPSVFLFPPKPKDTLMISRTPEVTCVVVDVSQEDPEVQF |
| | domain | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 173 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLYFRSLYYAMDYWGQG |
| | (M64V, | TLVTVSSASTKGPSVFPLAPCSRSTSESTAALGCLVKDYFP |
| | W101Y)/Human | EPVTVSWNSGALTSGVHTFPAVLQSSGLYSLSSVVTVPSS |
| | IgG4 (S228P) | SLGTKTYTCNVDHKPSNTKVDKRVESKYGPPCPPCPAPEF |
| | (K−) constant | LGGPSVFLFPPKPKDTLMISRTPEVTCVVVDVSQEDPEVQF |
| | domain | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 174 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLQFRSLYYAMDYWGQG |
| | (M64V, | TLVTVSSASTKGPSVFPLAPCSRSTSESTAALGCLVKDYFP |
| | 101Q)/Human | EPVTVSWNSGALTSGVHTFPAVLQSSGLYSLSSVVTVPSS |
| | IgG4 (S228P) | SLGTKTYTCNVDHKPSNTKVDKRVESKYGPPCPPCPAPEF |
| | (K−) constant | LGGPSVFLFPPKPKDTLMISRTPEVTCVVVDVSQEDPEVQF |
| | domain | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 175 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain | LYLQMNSLKAEDTAVYYCGRRLWFRSLYYAMDYWGQG |
| | VH2/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | (K−) constant | SLGTKTYTCNVDHKPSNTKVDKRVESKYGPPCPPCPAPEF |
| | domain | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 176 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH2 | LYLQMNSLKAEDTAVYYCGRRLWFRSLYYAMDYWGQG |
| | (M64V)/Human | TLVTVSSASTKGPSVFPLAPCSRSTSESTAALGCLVKDYFP |
| | IgG4 (S228P) | EPVTVSWNSGALTSGVHTFPAVLQSSGLYSLSSVVTVPSS |
| | (K−) constant | SLGTKTYTCNVDHKPSNTKVDKRVESKYGPPCPPCPAPEF |
| | domain | LGGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 177 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH2 | YLQMNSLKAEDTAVYYCGRRLWFRSLYYAMDYWGQGT |
| | (M64L)/Human | LVTVSSASTKGPSVFPLAPCSRSTSESTAALGCLVKDYFPE |
| | IgG4 (S228P) | PVTVSWNSGALTSGVHTFPAVLQSSGLYSLSSVVTVPSSS |
| | (K−) constant | LGTKTYTCNVDHKPSNTKVDKRVESKYGPPCPPCPAPEFL |
| | domain | GGPSVFLFPPKPKDTLMISRTPEVTCVVVDVSQEDPEVQF |
| | | NWYVDGVEVHNAKTKPREEQFNSTYRVVSVLTVLHQDW |
| | | LNGKEYKCKVSNKGLPSSIEKTISKAKGQPREPQVYTLPPS |
| | | QEEMTKNQVSLTCLVKGFYPSDIAVEWESNGQPENNYKT |
| | | TPPVLDSDGSFFLYSRLTVDKSRWQEGNVFSCSVMHEAL |
| | | HNHYTQKSLSLSLG |
| 178 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain VH1/ | LYLQMNSLRAEDTAVYYCGRRLWFRSLYYAMDYWGQG |
| | Human IgG1 HC | TLVTVSSASTKGPSVFPLAPSSKSTSGGTAALGCLVKDYFP |
| | (L234A, L235A, | EPVTVSWNSGALTSGVHTFPAVLQSSGLYSLSSVVTVPSS |
| | D265S) (K−) | SLGTQTYICNVNHKPSNTKVDKKVEPKSCDKTHTCPPCPA |
| | constant domain | PEAAGGPSVFLFPPKPKDTLMISRTPEVTCVVVSVSHEDPE |
| | | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPG |
| 179 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLWFRSLYYAMDYWGQG |
| | (M64V)/Human | TLVTVSSASTKGPSVFPLAPSSKSTSGGTAALGCLVKDYFP |
| | IgGI HC | EPVTVSWNSGALTSGVHTFPAVLQSSGLYSLSSVVTVPSS |
| | (L234A, L235A, | SLGTQTYICNVNHKPSNTKVDKKVEPKSCDKTHTCPPCPA |
| | D265S) (K−) | PEAAGGPSVFLFPPKPKDTLMISRTPEVTCVVVSVSHEDPE |
| | constant domain | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPG |
| 180 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH1 | YLQMNSLRAEDTAVYYCGRRLWFRSLYYAMDYWGQGT |
| | (M64L)/Human | LVTVSSASTKGPSVFPLAPSSKSTSGGTAALGCLVKDYFPE |
| | IgG1 HC | PVTVSWNSGALTSGVHTFPAVLQSSGLYSLSSVVTVPSSS |
| | (L234A, L235A, | LGTQTYICNVNHKPSNTKVDKKVEPKSCDKTHTCPPCPAP |
| | D265S) (K−) | EAAGGPSVFLFPPKPKDTLMISRTPEVTCVVVSVSHEDPEV |
| | constant domain | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPG |
| 181 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLFFRSLYYAMDYWGQGT |
| | (M64V, W101F)/ | LVTVSSASTKGPSVFPLAPSSKSTSGGTAALGCLVKDYFPE |
| | Human IgG1 HC | PVTVSWNSGALTSGVHTFPAVLQSSGLYSLSSVVTVPSSS |
| | (L234A, L235A, | LGTQTYICNVNHKPSNTKVDKKVEPKSCDKTHTCPPCPAP |
| | D265S) (K−) | EAAGGPSVFLFPPKPKDTLMISRTPEVTCVVVSVSHEDPEV |
| | constant domain | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPG |
| 182 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLYFRSLYYAMDYWGQG |
| | (M64V, | TLVTVSSASTKGPSVFPLAPSSKSTSGGTAALGCLVKDYFP |
| | W101Y)/Human | EPVTVSWNSGALTSGVHTFPAVLQSSGLYSLSSVVTVPSS |
| | IgG1 HC | SLGTQTYICNVNHKPSNTKVDKKVEPKSCDKTHTCPPCPA |
| | (L234A, L235A, | PEAAGGPSVFLFPPKPKDTLMISRTPEVTCVVVSVSHEDPE |
| | D265S) (K−) | VKFNWYVDGVEVHNAKTKPREEQYNSTYRVVSVLTVLH |
| | constant domain | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPG |
| 183 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLQFRSLYYAMDYWGQG |
| | (M64V, | TLVTVSSASTKGPSVFPLAPSSKSTSGGTAALGCLVKDYFP |
| | W101Q)/Human | EPVTVSWNSGALTSGVHTFPAVLQSSGLYSLSSVVTVPSS |
| | IgG1 HC | SLGTQTYICNVNHKPSNTKVDKKVEPKSCDKTHTCPPCPA |
| | (L234A, L235A, | PEAAGGPSVFLFPPKPKDTLMISRTPEVTCVVVSVSHEDPE |
| | D265S) (K−) | VKFNWYVDGVEVHNAKTKPREEQYNSTYRVVSVLTVLH |
| | constant domain | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPG |
| 184 | Humanized 52B8 | EVQLVESGGGLVQPGGSLRLSCAASGFTFSNYGMSWVRQ |
| | HC variable | APGKGLEWVATISGGGDYTNYPDSMRGRFTISRDNAKNS |
| | domain VH2/ | LYLQMNSLKAEDTAVYYCGRRLWFRSLYYAMDYWGQG |
| | Human IgG1 HC | TLVTVSSASTKGPSVFPLAPSSKSTSGGTAALGCLVKDYFP |
| | (L234A, L235A, | EPVTVSWNSGALTSGVHTFPAVLQSSGLYSLSSVVTVPSS |
| | D265S) (K−) | SLGTQTYICNVNHKPSNTKVDKKVEPKSCDKTHTCPPCPA |
| | constant domain | PEAAGGPSVFLFPPKPKDTLMISRTPEVTCVVVSVSHEDPE |
| | | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPG |
| 185 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH2 | LYLQMNSLKAEDTAVYYCGRRLWFRSLYYAMDYWGQG |
| | M64V/Human | TLVTVSSASTKGPSVFPLAPSSKSTSGGTAALGCLVKDYFP |
| | IgG1 HC | EPVTVSWNSGALTSGVHTFPAVLQSSGLYSLSSVVTVPSS |
| | (L234A, L235A, | SLGTQTYICNVNHKPSNTKVDKKVEPKSCDKTHTCPPCPA |
| | D265S) (K−) | PEAAGGPSVFLFPPKPKDTLMISRTPEVTCVVVSVSHEDPE |
| | constant domain | VKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPG |
| 186 | Humanized 52B8 | EVQLVESGGGLVQPGGSLRLSCAASGFTFSNYGMSWVRQ |
| | HC variable | APGKGLEWVATISGGGDYTNYPDSLRGRFTISRDNAKNSL |
| | domain VH2 | YLQMNSLKAEDTAVYYCGRRLWFRSLYYAMDYWGQGT |
| | M64L/Human | LVTVSSASTKGPSVFPLAPSSKSTSGGTAALGCLVKDYFPE |
| | IgGI HC | PVTVSWNSGALTSGVHTFPAVLQSSGLYSLSSVVTVPSSS |
| | (L234A, L235A, | LGTQTYICNVNHKPSNTKVDKKVEPKSCDKTHTCPPCPAP |
| | D265S) (K−) | EAAGGPSVFLFPPKPKDTLMISRTPEVTCVVVSVSHEDPEV |
| | constant domain | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPG |
| 187 | Chimeric Anti- | QVQLKESGPGLVQASETLSLTCTVSGFSLTSYSINWVRQSS |
| | ILT3 rat 40A6 | GKGPEWMGRFWYDEGIAYNLTLESRLSISGDTSKNQVFL |
| | parental HC | KMNSLRTGDTGTYYCTRDRDTVGITGWFAYWGQGTLVT |
| | variable | VSSASTKGPSVFPLAPSSKSTSGGTAALGCLVKDYFPEPVT |
| | domain/human | VSWNSGALTSGVHTFPAVLQSSGLYSLSSVVTVPSSSLGT |
| | IgG4 (S228P) | QTYICNVNHKPSNTKVDKKVEPKSCDKTHTCPPCPAPEAA |
| | constant domain | GGPSVFLFPPKPKDTLMISRTPEVTCVVVSVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPS |
| | | RDELTKNQVSLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| 188 | Chimeric Anti- | ETVMTQSPTSLSASIGERVTLNCKASQSVGVNVDWYQQT |
| | ILT3 rat 40A6 | PGQSPKLLIYGSANRHTGVPDRFTGSGFGSDFTLTISDVEP |
| | parental LC | EDLGVYYCLQYGSVPYTFGAGTKLELKRTVAAPSVFIFPP |
| | variable | SDEQLKSGTASVVCLLNNFYPREAKVQWKVDNALQSGN |
| | domain/human | SQESVTEQDSKDSTYSLSSTLTLSKADYEKHKVYACEVTH |
| | kappa | QGLSSPVTKSFNRGEC |
| 189 | Chimeric Anti- | QVQLKESGPGLVQASETLSLTCTVSGFSLTNYCVNWVRQ |
| | ILT3 rat 16B1 | PSGKGPEWLGRFWFDEGKAYNLTLESRLSISGDTSKNQVF |
| | parental HC | LRMNSLRADDTGTYYCTRDRDTVGITGWFAYWGQGTLV |
| | variable | TVSSASTKGPSVFPLAPSSKSTSGGTAALGCLVKDYFPEPV |
| | domain/human | TVSWNSGALTSGVHTFPAVLQSSGLYSLSSVVTVPSSSLG |
| | IgG4 (S228P) | TQTYICNVNHKPSNTKVDKKVEPKSCDKTHTCPPCPAPEA |
| | constant domain | AGGPSVFLFPPKPKDTLMISRTPEVTCVVVSVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 190 | Chimeric Anti- | ETVMTQSPTSLSASIGERVTLNCKASQSVGINVDWYQQTP |
| | ILT3 rat 16B1 | GQSPKLLIYGSANRHTGVPDRFTGSGFGSDFTLTISNVEPE |
| | parental LC | DLGVYYCLQYGSVPYTFGPGTKLELKRTVAAPSVFIFPPS |
| | variable | DEQLKSGTASVVCLLNNFYPREAKVQWKVDNALQSGNS |
| | domain/human | QESVTEQDSKDSTYSLSSTLTLSKADYEKHKVYACEVTH |
| | kappa | QGLSSPVTKSFNRGEC |
| 191 | Chimeric Anti- | QVQLQQSGAELMKPGASVKISCKATGYTFRTYWIEWVKQ |
| | ILT3 mouse | RPGHGLEWIGEILPGNGNTHENENFKDKATFTADTSSNAA |
| | 11D1 parental | YMQLSSLTSEDSAVYYCVRRLGRGPFDFWGQGTTLTVSS |
| | HC variable | ASTKGPSVFPLAPSSKSTSGGTAALGCLVKDYFPEPVTVS |
| | domain/human | WNSGALTSGVHTFPAVLQSSGLYSLSSVVTVPSSSLGTQT |
| | IgG4 (S228P) | YICNVNHKPSNTKVDKKVEPKSCDKTHTCPPCPAPEAAG |
| | constant domain | GPSVFLFPPKPKDTLMISRTPEVTCVVVSVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPS |
| | | RDELTKNQVSLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| 192 | Chimeric Anti- | DIQMTQSPSSLSVSLGGKVTITCKASQDINEYIGWYQRKP |
| | ILT3 mouse | GKGPRLLIHYTSTLQSGIPSRFSGSGSGRDYSLSISNLEPEDI |
| | 11D1 parental | ATYYCLQYANPLPTFGGGTKLEIKRTVAAPSVFIFPPSDEQ |
| | LC variable | LKSGTASVVCLLNNFYPREAKVQWKVDNALQSGNSQES |
| | domain/human | VTEQDSKDSTYSLSSTLTLSKADYEKHKVYACEVTHQGL |
| | kappa | SSPVTKSFNRGEC |
| 193 | Chimeric Anti- | EVQLVESGGGLVQPGRSMKLSCAASGFTFSNFDMAWVR |
| | ILT3 rat 17H12 | QAPTRGLEWVSSITYDGGSTSYRDSVKGRFTISRDNAKGT |
| | parental HC | LYLQMDSLRSEDTATYYCTTVESIATISTYFDYWGQGVM |
| | variable | VTVSSASTKGPSVFPLAPSSKSTSGGTAALGCLVKDYFPEP |
| | domain/human | VTVSWNSGALTSGVHTFPAVLQSSGLYSLSSVVTVPSSSL |
| | IgG4 (S228P) | GTQTYICNVNHKPSNTKVDKKVEPKSCDKTHTCPPCPAPE |
| | constant domain | AAGGPSVFLFPPKPKDTLMISRTPEVTCVVVSVSHEDPEV |
| | | KFNWYVDGVEVHNAKTKPREEQYNSTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 194 | Chimeric Anti- | DIVLTQSPALAVSLGQRATISCRASQSVSMSRYDLIHWYQ |
| | ILT3 rat 17H12 | QKPGQQPKLLIFRASDLASGIPARFSGSGSGTDFTLTINPVQ |
| | parental LC | ADDIATYYCQQTRKSPPTFGGGTRLELKRTVAAPSVFIFPP |
| | variable | SDEQLKSGTASVVCLLNNFYPREAKVQWKVDNALQSGN |
| | domain/human | SQESVTEQDSKDSTYSLSSTLTLSKADYEKHKVYACEVTH |
| | kappa | QGLSSPVTKSFNRGEC |
| 195 | Chimeric Anti- | QVQLKESGPGLVQASETLSLTCTVSGFSLTSYCVNWVRQP |
| | ILT3 rat 37C8 | SGKGPEWLGRFWYDEGKVYNLTLESRLSISGDTSKNQVF |
| | parental HC | LKMNRLRTDDTGTYYCTRDRDTMGITGWFAYWGQGTLV |
| | variable | TVSSASTKGPSVFPLAPSSKSTSGGTAALGCLVKDYFPEPV |
| | domain/human | TVSWNSGALTSGVHTFPAVLQSSGLYSLSSVVTVPSSSLG |
| | IgG4 (S228P) | TQTYICNVNHKPSNTKVDKKVEPKSCDKTHTCPPCPAPEA |
| | constant domain | AGGPSVFLFPPKPKDTLMISRTPEVTCVVVSVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 196 | Chimeric Anti- | ETVMTQSPTSLSASIGERVTLNCKASQSVGINVDWYQQTP |
| | ILT3 rat 37C8 | GQSPKLLIYGSANRHTGVPDRFTGSGFGSGFTLTISNVEPE |
| | parental LC | DLGVYYCLQYGSVPYTFGPGTKLELKRTVAAPSVFIFPPS |
| | variable | DEQLKSGTASVVCLLNNFYPREAKVQWKVDNALQSGNS |
| | domain/human | QESVTEQDSKDSTYSLSSTLTLSKADYEKHKVYACEVTH |
| | kappa | QGLSSPVTKSFNRGEC |
| 197 | Chimeric Anti- | QVQMQQSGTELMKPGASMKISCKATGYTFSTYWIQWIKQ |
| | ILT3 mouse | RPGHGLEWIGEILPGSGTTNYNENFKGKATFSADTSSNTA |
| | 1G12 parental | YIHLSSLTSEDSAVFYCARRLGRGPFDYWGQGTTLTVSSA |
| | HC variable | STKGPSVFPLAPSSKSTSGGTAALGCLVKDYFPEPVTVSW |
| | domain/human | NSGALTSGVHTFPAVLQSSGLYSLSSVVTVPSSSLGTQTYI |
| | IgG4 (S228P) | CNVNHKPSNTKVDKKVEPKSCDKTHTCPPCPAPEAAGGP |
| | constant domain | SVFLFPPKPKDTLMISRTPEVTCVVVSVSHEDPEVKFNWY |
| | | VDGVEVHNAKTKPREEQYNSTYRVVSVLTVLHQDWLNG |
| | | KEYKCKVSNKALPAPIEKTISKAKGQPREPQVYTLPPSRDE |
| | | LTKNQVSLTCLVKGFYPSDIAVEWESNGQPENNYKTTPPV |
| | | LDSDGSFFLYSKLTVDKSRWQQGNVFSCSVMHEALHNHY |
| | | TQKSLSLSPGK |
| 198 | Chimeric Anti- | DIQMTQSPSSLSASLGGKVTITCEASQDINKHIDWYQHQP |
| | ILT3 mouse | GRGPSLLIHYASILQPGIPSRFSGSGSGRDYSFSITSLEPEDI |
| | 1G12 parental | ATYYCLQYDNLLPTFGGGTKLEIKRTVAAPSVFIFPPSDEQ |
| | LC variable | LKSGTASVVCLLNNFYPREAKVQWKVDNALQSGNSQES |
| | domain/human | VTEQDSKDSTYSLSSTLTLSKADYEKHKVYACEVTHQGL |
| | kappa | SSPVTKSFNRGEC |
| 199 | Chimeric Anti- | QVQLKESGPGLVQASETLSLTCTVSGFSLTSYSVNWVRQP |
| | ILT3 rat 20E4 | SGKGLEWMGRFWYDGGTAYNSTLESRLSISGDTSKNQVF |
| | parental HC | LKMNSLQTDDTGTYYCTRDRDTMGITGWFAYWGQGTLV |
| | variable | TVSPASTKGPSVFPLAPSSKSTSGGTAALGCLVKDYFPEPV |
| | domain/human | TVSWNSGALTSGVHTFPAVLQSSGLYSLSSVVTVPSSSLG |
| | IgG4 (S228P) | TQTYICNVNHKPSNTKVDKKVEPKSCDKTHTCPPCPAPEA |
| | constant domain | AGGPSVFLFPPKPKDTLMISRTPEVTCVVVSVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 200 | Chimeric Anti- | ETVMTQSPTSLSASIGERVTLNCKASQSVGVNVDWYQQT |
| | ILT3 rat 20E4 | PGQSPKLLIYGSANRHTGVPDRFTGSGFGSDFTLTISNVEP |
| | parental LC | EDLGVYYCLQYGSVPYTFGAGTKLELKRTVAAPSVFIFPP |
| | variable | SDEQLKSGTASVVCLLNNFYPREAKVQWKVDNALQSGN |
| | domain/human | SQESVTEQDSKDSTYSLSSTLTLSKADYEKHKVYACEVTH |
| | kappa | QGLSSPVTKSFNRGEC |
| 201 | Chimeric Anti- | QVQLKESGPGLVQASETLSLTCTVSGFSLTSYCVNWVRQP |
| | ILT3 rat 24A4 | SGKGPEWLGRFWYDEGKVYNLTLESRLSISGDTSKNQVF |
| | parental HC | LKMNRLRTDDTGTYYCTRDRDTLGITGWFAYWGQGTLV |
| | variable | TVSSASTKGPSVFPLAPSSKSTSGGTAALGCLVKDYFPEPV |
| | domain/human | TVSWNSGALTSGVHTFPAVLQSSGLYSLSSVVTVPSSSLG |
| | IgG4 (S228P) | TQTYICNVNHKPSNTKVDKKVEPKSCDKTHTCPPCPAPEA |
| | constant domain | AGGPSVFLFPPKPKDTLMISRTPEVTCVVVSVSHEDPEVKF |
| | | NWYVDGVEVHNAKTKPREEQYNSTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 202 | Chimeric Anti- | ETVMTQSPTSLSASIGERVTLNCKASQSVGINVDWYQQTP |
| | ILT3 rat 24A4 | GQSPKLLIYGSANRHTGVPDRFTGSGFGSGFTLTISNVEPE |
| | parental LC | DLGVYYCLQYGSVPYTFGPGTKLELKRTVAAPSVFIFPPS |
| | variable | DEQLKSGTASVVCLLNNFYPREAKVQWKVDNALQSGNS |
| | domain/human | QESVTEQDSKDSTYSLSSTLTLSKADYEKHKVYACEVTH |
| | kappa | QGLSSPVTKSFNRGEC |
| 203 | Humanized 52B8 | EVQLVESGGGLVQPGGSLRLSCAASGFTFSNYGMSWVRQ |
| | HC variable | APGKGLEWVATISGGGDYTNYPDSVRGRFTISRDNAKNS |
| | domain VH1 | LYLQMNSLRAEDTAVYYCGRRLWFRSLYYAMDYWGQG |
| | (M64V)/Human | TLVTVSSASTKGPSVFPLAPSSKSTSGGTAALGCLVKDYFP |
| | IgG1 HC | EPVTVSWNSGALTSGVHTFPAVLQSSGLYSLSSVVTVPSS |
| | (N297A) | SLGTQTYICNVNHKPSNTKVDKKVEPKSCDKTHTCPPCPA |
| | constant domain | PELLGGPSVFLFPPKPKDTLMISRTPEVTCVVVDVSHEDPE |
| | | VKFNWYVDGVEVHNAKTKPREEQYASTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| 204 | Human IgG1 HC | ASTKGPSVFPLAPSSKSTSGGTAALGCLVKDYFPEPVTVS |
| | constant domain | WNSGALTSGVHTFPAVLQSSGLYSLSSVVTVPSSSLGTQT |
| | (N297A) | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYASTYRVVSVLTVLHQDWLN |
| | | GKEYKCKVSNKALPAPIEKTISKAKGQPREPQVYTLPPSR |
| | | DELTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 205 | Chimeric anti- | QVQLKESGPGLVQASETLSLTCTVSGFSLTSYSINWVRQSS |
| | ILT3 40A6 rat | GKGPEWMGRFWYDEGIAYNLTLESRLSISGDTSKNQVFL |
| | VH/human IgG1 | KMNSLRTGDTGTYYCTRDRDTVGITGWFAYWGQGTLVT |
| | (N297A) | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 206 | Chimeric anti- | QVQLKESGPGLVQASETLSLTCTVSGFSLTNYCVNWVRQ |
| | ILT3 16B1 rat | PSGKGPEWLGRFWFDEGKAYNLTLESRLSISGDTSKNQVF |
| | VH/human IgG1 | LRMNSLRADDTGTYYCTRDRDTVGITGWFAYWGQGTLV |
| | (N297A) | TVSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 207 | Chimeric anti- | QVQLQQSGAELMKPGASVKISCKATGYTFRTYWIEWVKQ |
| | ILT3 11D1 | RPGHGLEWIGEILPGNGNTHFNENFKDKATFTADTSSNAA |
| | mouse VH/ | YMQLSSLTSEDSAVYYCVRRLGRGPFDFWGQGTTLTVSS |
| | human IgG1 | ASTKGPSVFPLAPSSKSTSGGTAALGCLVKDYFPEPVTVS |
| | (N297A) | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYASTYRVVSVLTVLHQDWLN |
| | | GKEYKCKVSNKALPAPIEKTISKAKGQPREPQVYTLPPSR |
| | | DELTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 208 | Chimeric anti- | EVQLVESGGGLVQPGRSMKLSCAASGFTFSNFDMAWVR |
| | ILT3 17H12 rat | QAPTRGLEWVSSITYDGGSTSYRDSVKGRFTISRDNAKGT |
| | VH/human IgG1 | LYLQMDSLRSEDTATYYCTTVESIATISTYFDYWGQGVM |
| | (N297A) | VTVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKKVEPKSCDKTHTCPPCPAPE |
| | | LLGGPSVFLFPPKPKDTLMISRTPEVTCVVVDVSHEDPEV |
| | | KFNWYVDGVEVHNAKTKPREEQYASTYRVVSVLTVLHQ |
| | | DWLNGKEYKCKVSNKALPAPIEKTISKAKGQPREPQVYT |
| | | LPPSRDELTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 209 | Chimeric anti- | QVQLKESGPGLVQASETLSLTCTVSGFSLTSYCVNWVRQP |
| | ILT3 37C8 rat | SGKGPEWLGRFWYDEGKVYNLTLESRLSISGDTSKNQVF |
| | VH/human IgG1 | LKMNRLRTDDTGTYYCTRDRDTMGITGWFAYWGQGTLV |
| | (N297A) | TVSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 210 | Chimeric anti- | QVQMQQSGTELMKPGASMKISCKATGYTFSTYWIQWIKQ |
| | ILT3 1G12 | RPGHGLEWIGEILPGSGTTNYNENFKGKATFSADTSSNTA |
| | mouse VH/ | YIHLSSLTSEDSAVFYCARRLGRGPFDYWGQGTTLTVSSA |
| | human IgG1 | STKGPSVFPLAPSSKSTSGGTAALGCLVKDYFPEPVTVSW |
| | (N297A) | NSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLMISRTPEVTCVVVDVSHEDPEVKFNWYV |
| | | DGVEVHNAKTKPREEQYASTYRVVSVLTVLHQDWLNGK |
| | | EYKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGK |
| 211 | Chimeric anti- | QVQLKESGPGLVQASETLSLTCTVSGFSLTSYSVNWVRQP |
| | ILT3 20E4 rat | SGKGLEWMGRFWYDGGTAYNSTLESRLSISGDTSKNQVF |
| | VH/human IgG1 | LKMNSLQTDDTGTYYCTRDRDTMGITGWFAYWGQGTLV |
| | (N297A) | TVSPASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 212 | Chimeric anti- | QVQLKESGPGLVQASETLSLTCTVSGFSLTSYCVNWVRQP |
| | ILT3 24A4 rat | SGKGPEWLGRFWYDEGKVYNLTLESRLSISGDTSKNQVF |
| | VH/human IgG1 | LKMNRLRTDDTGTYYCTRDRDTLGITGWFAYWGQGTLV |
| | (N297A) | TVSSASTKGPSVFPLAPSSKSTSGGTAALGCLVKDYFPEPV |
| | | TVSWNSGALTSGVHTFPAVLQSSGLYSLSSVVTVPSSSLG |
| | | TQTYICNVNHKPSNTKVDKKVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| 213 | Chimeric anti- | QVQLKESGPGLVQASETLSLTCTVSGFSLTSYSINWVRQSS |
| | ILT3 40A6 rat | GKGPEWMGRFWYDEGIAYNLTLESRLSISGDTSKNQVFL |
| | VH/human IgG1 | KMNSLRTGDTGTYYCTRDRDTVGITGWFAYWGQGTLVT |
| | (N297A) | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKF |
| | | NWYVDGVEVHNAKTKPREEQYASTYRVVSVLTVLHQD |
| | | WLNGKEYKCKVSNKALPAPIEKTISKAKGQPREPQVYTLP |
| | | PSRDELTKNQVSLTCLVKGFYPSDIAVEWESNGQPENNYK |
| | | TTPPVLDSDGSFFLYSKLTVDKSRWQQGNVFSCSVMHEA |
| | | LHNHYTQKSLSLSPGK |
| | Residues after | FGXG |
| | LC-CDR3 | |
| | Xaa is any amino | |
| | acid | |
| | Residues before | CXXX |
| | HC-CDR1 | |
| | Xaa is any amino | |
| | acid | |
| 216 | Residues before | LEWIG |
| | HC-CDR1 | |
| | Residues after | WGXG |
| | HC-CDR3 | |
| | Xaa is any | |
| | residue | |
| 218 | Human IgG1 HC | ASTKGPSVFPLAPSSKSTSGGTAALGCLVKDYFPEPVTVS |
| | constant domain | WNSGALTSGVHTFPAVLQSSGLYSLSSVVTVPSSSLGTQT |
| | Residue 148 | YICNVNHKPSNTKVDKKVEPKSCDKTHTCPPCPAPELLGG |
| | corresponds with | PSVFLFPPKPKDTLMISRTPEVTCVVVAVSHEDPEVKFNW |
| | N297A, residue | YVDGVEVHNAKTKPREEQYASTYRVVSVLTVLHQDWLN |
| | 180 corresponds | GKEYKCKVSNKALPAPIEKTISKAKGQPREPQVYTLPPSR |
| | with D265A | DELTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| | Residues after | FGXG |
| | LC-CDR3 | |
| | Xaa is any amino | |
| | acid | |
| | Residues before | CXXX |
| | HC-CDR1 | |
| | Xaa is any amino | |
| | acid | |
| 221 | Residues before | LEWIG |
| | HC-CDR1 | |
| | Residues after | WGXG |
| | HC-CDR3 | |
| | Xaa is any | |
| | residue | |
| 223 | Human IgG1 HC | ASTKGPSVFPLAPSSKSTSGGTAALGCLVKDYFPEPVTVS |
| | constant domain | WNSGALTSGVHTFPAVLQSSGLYSLSSVVTVPSSSLGTQT |
| | Residue 148 | YICNVNHKPSNTKVDKKVEPKSCDKTHTCPPCPAPELLGG |
| | corresponds with | PSVFLFPPKPKDTLMISRTPEVTCVVVAVSHEDPEVKFNW |
| | N297A, residue | YVDGVEVHNAKTKPREEQYASTYRVVSVLTVLHQDWLN |
| | 180 corresponds | GKEYKCKVSNKALPAPIEKTISKAKGQPREPQVYTLPPSR |
| | with D265A | DELTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |

In specific embodiments, the treatment methods and uses of the present invention provides the anti-ILT3 antibodies shown in Table 4 below. With the exception of those antibodies comprising a replacement of the tryptophan residue at position 101 of the V_{H}, the antibodies disclosed herein bind human ILT3.

TABLE 4
Exemplary anti-ILT3 antibodies

| | | SEQ ID NO: | |
|---|---|---|---|
| | | Heavy | Light |
| mAb No. | Description | Chain | Chain |
| 1 | Humanized anti-ILT3 mAb (52B8 VH1/VL1) | 135 | 144 |
| | IgG4 S228P/Kappa | | |
| 2 | Humanized anti-ILT3 mAb (52B8 VH1/VL2) | 135 | 145 |
| | IgG4 S228P/Kappa | | |
| 3 | Humanized anti-ILT3 mAb (52B8 VH1/VL3) | 135 | 146 |
| | IgG4 S228P/Kappa | | |
| 4 | Humanized anti-ILT3 mAb (52B8 VH1/VL4) | 135 | 147 |
| | IgG4 S228P/Kappa | | |
| 5 | Humanized anti-ILT3 mAb (52B8 VH2/VL1) | 141 | 144 |
| | IgG4 S228P/Kappa | | |
| 6 | Humanized anti-ILT3 mAb (52B8 VH2/VL2) | 141 | 145 |
| | IgG4 S228P/Kappa | | |
| 7 | Humanized anti-ILT3 mAb (52B8 VH2/VL3) | 141 | 146 |
| | IgG4 S228P/Kappa | | |
| 8 | Humanized anti-ILT3 mAb (52B8 VH2/VL4) | 141 | 147 |
| | IgG4 S228P/Kappa | | |
| 9 | Humanized anti-ILT3 mAb (52B8 VH1 | 136 | 144 |
| | M64V/VL1) IgG4 S228P/Kappa | | |
| 10 | Humanized anti-ILT3 mAb (52B8 VH1 | 136 | 145 |
| | M64V/VL2) IgG4 S228P/Kappa | | |
| 11 | Humanized anti-ILT3 mAb (52B8 VH1 | 136 | 146 |
| | M64V/VL3) IgG4 S228P/Kappa | | |
| 12 | Humanized anti-ILT3 mAb (52B8 VH1 | 136 | 147 |
| | M64V/VL4) IgG4 S228P/Kappa | | |
| 13 | Humanized anti-ILT3 mAb (52B8 VH2 | 142 | 144 |
| | M64V/VL1) IgG4 S228P/Kappa | | |
| 14 | Humanized anti-ILT3 mAb (52B8 VH2 | 142 | 145 |
| | M64V/VL2) IgG4 S228P/Kappa | | |
| 15 | Humanized anti-ILT3 mAb (52B8 VH2 | 142 | 146 |
| | M64V/VL3) IgG4 S228P/Kappa | | |
| 16 | Humanized anti-ILT3 mAb (52B8 VH2 | 142 | 147 |
| | M64V/VL4) IgG4 S228P/Kappa | | |
| 17 | Humanized anti-ILT3 mAb (52B8 VH1 | 137 | 144 |
| | M64L/VL1) IgG4 S228P/Kappa | | |
| 18 | Humanized anti-ILT3 mAb (52B8 VH1 | 137 | 145 |
| | M64L/VL2) IgG4 S228P/Kappa | | |
| 19 | Humanized anti-ILT3 mAb (52B8 VH1 | 137 | 146 |
| | M64L/VL3) IgG4 S228P/Kappa | | |
| 20 | Humanized anti-ILT3 mAb (52B8 VH1 | 137 | 148 |
| | M64L/VL4) IgG4 S228P/Kappa | | |
| 21 | Humanized anti-ILT3 mAb (52B8 VH2 | 143 | 144 |
| | M64L/VL1) IgG4 S228P/Kappa | | |
| 22 | Humanized anti-ILT3 mAb (52B8 VH2 | 143 | 145 |
| | M64L/VL2) IgG4 S228P/Kappa | | |
| 23 | Humanized anti-ILT3 mAb (52B8 VH2 | 143 | 146 |
| | M64L/VL3) IgG4 S228P/Kappa | | |
| 24 | Humanized anti-ILT3 mAb (52B8 VH2 | 143 | 147 |
| | M64L/VL4) IgG4 S228P/Kappa | | |
| 25 | Humanized anti-ILT3 mAb ((52B8 VH1 | 162 | 145 |
| | M64V/VL2) L234A L235A D265S) | | |
| | IgG1/Kappa | | |
| 26 | Humanized anti-ILT3 mAb ((52B8 VH1 | 162 | 148 |
| | M64V/VL5) L234A L235A D265S) | | |
| | IgG1/Kappa | | |
| 27 | Humanized anti-ILT3 mAb ((52B8 VH1 | 162 | 149 |
| | M64V/VL6) L234A L235A D265S) | | |
| | IgG1/Kappa | | |
| 28 | Humanized anti-ILT3 mAb ((52B8 VH1 | 152 | 150 |
| | M64V/VL7) L234A L235A D265S) | | |
| | IgG1/Kappa | | |
| 29 | Humanized anti-ILT3 mAb ((52B8 VH1 | 162 | 151 |
| | M64V/VL8) L234A L235A D265S) | | |
| | IgG1/Kappa | | |
| 30 | Humanized anti-ILT3 mAb (52B8 VH1 | 136 | 148 |
| | M64V/VL5) IgG4 S228P/Kappa | | |
| 31 | Humanized anti-ILT3 mAb (52B8 VH1 | 136 | 149 |
| | M64V/VL6) IgG4 S228P/Kappa | | |
| 32 | Humanized anti-ILT3 mAb (52B8 VH1 | 136 | 150 |
| | M64V/VL7) IgG4 S228P/Kappa | | |
| 33 | Humanized anti-ILT3 mAb (52B8 VH1 | 136 | 151 |
| | M64V/VL8) IgG4 S228P/Kappa | | |
| 34 | Humanized anti-ILT3 mAb (52B8 VH1 | 138 | 145 |
| | M64V W101F/VL2) IgG4 S228P/Kappa | | |
| 35 | Humanized anti-ILT3 mAb (52B8 VH1 | 139 | 145 |
| | M64V W101Y/VL2) IgG4 S228P/Kappa | | |
| 36 | Humanized anti-ILT3 mAb (52B8 VH1 | 140 | 145 |
| | M64V W101Q/VL2) IgG4 S228P/Kappa | | |
| 37 | Humanized anti-ILT3 mAb ((52B8 VH1 | 138 | 145 |
| | M64V W101F/VL2) L234A L235A D265S) | | |
| | IgG1/Kappa | | |
| 38 | Humanized anti-ILT3 mAb ((52B8 VH1 | 139 | 145 |
| | M64V W101Y/VL2) L234A L235A D265S) | | |
| | IgG1/Kappa | | |
| 39 | Humanized anti-ILT3 mAb ((52B8 VH1 | 140 | 145 |
| | M64V W101Q/VL2) L234A L235A D265S) | | |
| | IgG1/Kappa | | |
| 40 | Humanized anti-ILT3 mAb (52B8 VH1 | 136 | 152 |
| | M64V/VL2 S35A) IgG4 S228P/Kappa | | |
| 41 | Humanized anti-ILT3 mAb (52B8 VH1 | 136 | 153 |
| | M64V/VL2 S35N) IgG4 S228P/Kappa | | |
| 42 | Humanized anti-ILT3 mAb (52B8 VH1 | 136 | 154 |
| | M64V/VL2 N34Q) IgG4 S228P/Kappa | | |
| 43 | Humanized anti-ILT3 mAb (52B8 VH1 | 136 | 155 |
| | M64V/VL2 N34D) IgG4 S228P/Kappa | | |
| 44 | Humanized anti-ILT3 mAb (52B8 VH1 | 136 | 156 |
| | M64V/VL5 S35A) IgG4 S228P/Kappa | | |
| 45 | Humanized anti-ILT3 mAb (52B8 VH1 | 136 | 157 |
| | M64V/VL5 S35N) IgG4 S228P/Kappa | | |
| 46 | Humanized anti-ILT3 mAb (52B8 VH1 | 136 | 158 |
| | M64V/VL5 N34Q) IgG4 S228P/Kappa | | |
| 47 | Humanized anti-ILT3 mAb (52B8 VH1 | 136 | 159 |
| | M64V/VL5 N34D) IgG4 S228P/Kappa | | |
| 48 | Humanized anti-ILT3 mAb (52B8 VH1 | 138 | 148 |
| | M64V W101F/VL5) IgG4 S228P/Kappa | | |
| 49 | Humanized anti-ILT3 mAb (52B8 VH1 | 139 | 148 |
| | M64V W101Y/VL5) IgG4 S228P/Kappa | | |
| 50 | Humanized anti-ILT3 mAb (52B8 VH1 | 140 | 148 |
| | M64V W101Q/VL5) IgG4 S228P/Kappa | | |
| 51 | Humanized anti-ILT3 mAb (52B8 VH1 | 138 | 156 |
| | M64V W101F/VL5 S35A) IgG4 S228P/Kappa | | |
| 52 | Humanized anti-ILT3 mAb (52B8 VH1 | 138 | 157 |
| | M64V W101F/VL5 S35N) IgG4 S228P/Kappa | | |
| 53 | Humanized anti-ILT3 mAb (52B8 VH1 | 138 | 158 |
| | M64V W101F/VL5 N34Q) IgG4 S228P/Kappa | | |
| 54 | Humanized anti-ILT3 mAb (52B8 VH1 | 138 | 159 |
| | M64V W101F/VL5 N34D) IgG4 S228P/Kappa | | |
| 55 | Humanized anti-ILT3 mAb (52B8 VH1 | 139 | 156 |
| | M64V W101Y/VL5 S35A) IgG4 S228P/Kappa | | |
| 56 | Humanized anti-ILT3 mAb (52B8 VH1 | 139 | 157 |
| | M64V W101Y/VL5 S35N) IgG4 S228P/Kappa | | |
| 57 | Humanized anti-ILT3 mAb (52B8 VH1 | 139 | 158 |
| | M64V W101Y/VL5 N34Q) IgG4 S228P/Kappa | | |
| 58 | Humanized anti-ILT3 mAb (52B8 VH1 | 139 | 159 |
| | M64V W101Y/VL5 N34D) IgG4 S228P/Kappa | | |
| 59 | Humanized anti-ILT3 mAb (52B8 VH1 | 140 | 156 |
| | M64V W101Q/VL5 S35A) IgG4 S228P/Kappa | | |
| 60 | Humanized anti-ILT3 mAb (52B8 VH1 | 140 | 157 |
| | M64V W101Q/VL5 S35N) IgG4 S228P/Kappa | | |
| 61 | Humanized anti-ILT3 mAb (52B8 VH1 | 140 | 158 |
| | M64V W101Q/VL5 N34Q) IgG4 S228P/Kappa | | |
| 62 | Humanized anti-ILT3 mAb (52B8 VH1 | 140 | 159 |
| | M64V W101Q/VL5 N34D) IgG4 S228P/Kappa | | |
| 63 | Humanized anti-ILT3 mAb (52B8 VH1 | 203 | 119 |
| | M64V/VL1 N34Q) IgG1 N297A/Kappa | | |
| 64 | Humanized anti-ILT3 mAb (52B8 VH1 | 203 | 120 |
| | M64V/VL2 IgG1 N297A/Kappa | | |
| 65 | Humanized anti-ILT3 mAb (52B8 VH1 | 203 | 154 |
| | M64V/VL2 N34Q) IgG1 N297A/Kappa | | |
| 66 | Humanized anti-ILT3 mAb (52B8 VH1 | 203 | 121 |
| | M64V/VL3 N34Q) IgG1 N297A/Kappa | | |
| 67 | Humanized anti-ILT3 mAb (52B8 VH1 | 203 | 122 |
| | M64V/VL4 N34Q) IgG1 N297A/Kappa | | |
| 68 | Humanized anti-ILT3 mAb (52B8 VH1 | 204 | 123 |
| | M64V/VL5 IgG1 N297A/Kappa | | |
| 69 | Humanized anti-ILT3 mAb (52B8 VH1 | 203 | 158 |
| | M64V/VL5 N34Q) IgG1 N297A/Kappa | | |
| 70 | Humanized anti-ILT3 mAb (52B8 VH1 | 203 | 124 |
| | M64V/VL6 N34Q) IgG1 N297A/Kappa | | |
| 71 | Humanized anti-ILT3 mAb (52B8 VH1 | 203 | 125 |
| | M64V/VL7 N34Q) IgG1 N297A/Kappa | | |
| 72 | Humanized anti-ILT3 mAb (52B8 VH1 | 203 | 126 |
| | M64V/VL8 N34Q) IgG1 N297A/Kappa | | |
| 73 | Chimeric anti-ILT3 52B8 mouse | 106 | 109 |
| | VH/human IgG4 (S228P): mouse | | |
| | VL/human Kappa | | |
| 74 | Chimeric anti-ILT3 52B8 mouse VH | 107 | 109 |
| | M64V/human IgG4 (S228P): mouse | | |
| | VL/human Kappa | | |
| 75 | Chimeric anti-ILT3 52B8 mouse VH | 108 | 109 |
| | M64L/human IgG4 (S228P): mouse | | |
| | VL/human Kappa | | |
| 76 | Chimeric anti-ILT3 52B8 mouse | Residues 1-122 | 109 |
| | VH/human IgG1 (N297A): mouse | of SEQ ID | |
| | VL/human Kappa | NO: 106 | |
| | | And SEQ ID | |
| | | NO: 204 | |
| 77 | Chimeric anti-ILT3 52B8 mouse VH | Residues 1-122 | 109 |
| | M64V/human IgG1 (N297A): mouse | of SEQ ID | |
| | VL/human Kappa | NO: 107 | |
| | | And SEQ ID | |
| | | NO: 204 | |
| 78 | Chimeric anti-ILT3 52B8 mouse | Residues 1-122 | 109 |
| | VH/human IgG1: mouse | of SEQ ID | |
| | VL/human Kappa | NO: 106 | |
| | | And SEQ ID | |
| | | NO: 4 | |
| 79 | Chimeric anti-ILT3 52B8 mouse VH | Residues 1-122 | 109 |
| | M64V/human IgG1: mouse | of SEQ ID | |
| | VL/human Kappa | NO: 107 | |
| | | And SEQ ID | |
| | | NO: 4 | |
| 80 | Chimeric anti-ILT3 40A6 rat VH/human | 187 | 188 |
| | IgG4 (S228P): rat VL/human Kappa | | |
| 81 | Chimeric anti-ILT3 16B1 rat VH/human | 189 | 190 |
| | IgG4 (S228P): rat VL/human Kappa | | |
| 82 | Chimeric anti-ILT3 11D1 mouse VH/human | 191 | 192 |
| | IgG4 (S228P): mouse VL/human Kappa | | |
| 83 | Chimeric anti-ILT3 17H12 rat VH/human | 193 | 194 |
| | IgG4 (S228P): rat VL/human Kappa | | |
| 84 | Chimeric anti-ILT3 37C8 rat VH/human | 195 | 196 |
| | IgG4 (S228P): rat VL/human Kappa | | |
| 85 | Chimeric anti-ILT3 1G12 mouse VH/human | 197 | 198 |
| | IgG4 (S228P): mouse VL/human Kappa | | |
| 86 | Chimeric anti-ILT3 20E4 rat VH/human | 199 | 200 |
| | IgG4 (S228P): rat VL/human Kappa | | |
| 87 | Chimeric anti-ILT3 24A4 rat VH/human | 201 | 202 |
| | IgG4 (S228P): rat VL/human Kappa | | |
| 88 | Chimeric anti-ILT3 40A6 rat VH/human | 205 | 188 |
| | IgG1 (N297A): rat VL/human Kappa | | |
| 89 | Chimeric anti-ILT3 16B1 rat VH/human | 206 | 190 |
| | IgG1 (N297A): rat VL/human Kappa | | |
| 90 | Chimeric anti-ILT3 11D1 mouse VH/human | 207 | 192 |
| | IgG1 (N297A): mouse VL/human Kappa | | |
| 91 | Chimeric anti-ILT3 17H12 rat VH/human | 208 | 194 |
| | IgG1 (N297A): rat VL/human Kappa | | |
| 92 | Chimeric anti-ILT3 37C8 rat VH/human | 209 | 196 |
| | IgG1 (N297A): rat VL/human Kappa | | |
| 93 | Chimeric anti-ILT3 1G12 mouse VH/human | 210 | 198 |
| | IgG1 (N297A): mouse VL/human Kappa | | |
| 94 | Chimeric anti-ILT3 20E4 rat VH/human | 211 | 200 |
| | IgG1 (N297A): rat VL/human Kappa | | |
| 95 | Chimeric anti-ILT3 24A4 rat VH/human | 212 | 202 |
| | IgG1 (N297A): rat VL/human Kappa | | |
| 96 | Chimeric anti-ILT3 40A6 rat VH/human | 210 | 188 |
| | IgG1 (N297A): rat VL/human Kappa | | |

In particular embodiments of the invention, the anti-ILT3 antigen binding protein or fragment is a human or humanized anti-ILT3 antibody or antigen binding fragment or a chimeric anti-ILT3 antibody or antigen binding fragment that comprises HC-CDR1, HC-CDR2, HC-CDR3, LC-CDR1, LC-CDR2, and LC-CDR3 of an anti-ILT3 antibody molecule disclosed herein or in Table 5 below.

TABLE 5
ILT3 antibody CDRs

| | | SEQ | | SEQ | | SEQ |
|---|---|---|---|---|---|---|
| | | ID | | ID | | ID |
| mAb | HC-CDR1 | NO. | HC-CDR2 | NO. | HC-CDR3 | NO. |
| 52B8 | NYGMS | 10 | TISGGGDYTNY | 13 | RLWFRSLYYAM | 16 |
| | | | PDSVRG | | DY | |
| 40A6 | SYSIN | 40 | RFWYDEGIAYN | 41 | DRDTVGITGWF | 42 |
| | | | LTLES | | AY | |
| 16B1 | NYCVN | 48 | RFWFDEGKAYN | 49 | DRDTVGITGWF | 50 |
| | | | LTLES | | AY | |
| 11D1 | TYWIE | 56 | EILPGNGNTHEN | 57 | RRLGRGPFDF | 58 |
| | | | ENFKD | | | |
| 17H12 | NFDMA | 64 | SITYDGGSTSYR | 65 | VESIATISTYFD | 66 |
| | | | DSVKG | | Y | |
| 37C8 | SYCVN | 72 | RFWYDEGKVY | 73 | DRDTMGITGWF | 74 |
| | | | NLTLES | | AY | |
| 1G12 | TYWIQ | 80 | EILPGSGTTNYN | 81 | RLGRGPFDY | 82 |
| | | | ENFKG | | | |
| 20E4 | SYSVN | 88 | RFWYDGGTAY | 89 | DRDTMGITGWF | 90 |
| | | | NSTLES | | AY | |
| 24A4 | SYCVN | 96 | RFWYDEGKVY | 97 | DRDTLGITGWF | 98 |
| | | | NLTLES | | AY | |
| mAb | LC-CDR1 | | LC-CDR2 | | LC-CDR3 | |
| 52B8 | RASEKVDS | 34 | LTSNLDS | 36 | QQNNEDPYT | 37 |
| | FGQSFMH | | | | | |
| 40A6 | KASQSVGV | 43 | GSANRHT | 44 | LQYGSVPYT | 45 |
| | NVD | | | | | |
| 16B1 | KASQSVGI | 51 | GSANRHT | 52 | LQYGSVPYT | 53 |
| | NVD | | | | | |
| 11D1 | KASQDINE | 59 | YTSTLQS | 60 | LQYANPLPT | 61 |
| | YIG | | | | | |
| 17H12 | RASQSVSM | 67 | RASDLAS | 68 | QQTRKSPPT | 69 |
| | SRYDLIH | | | | | |
| 37C8 | KASQSVGI | 75 | GSANRHT | 76 | LQYGSVPYT | 77 |
| | NVD | | | | | |
| 1G12 | EASQDINK | 83 | YASILQP | 84 | LQYDNLLPT | 85 |
| | HID | | | | | |
| 20E4 | KASQSVGV | 91 | GSANRHT | 92 | LQYGSVPYT | 93 |
| | NVD | | | | | |
| 24A4 | KASQSVGI | 99 | GSANRHT | 100 | LQYGSVPYT | 101 |
| | NVD | | | | | |

### Anti-PD-1 Antigen Binding Proteins and Antigen Binding Fragments Useful in the Invention

An “anti-PD-1 antigen binding protein or antigen binding fragment” useful in the any of the treatment methods, compositions and uses of the present invention include monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, compositions and uses of the present invention in which a human individual is being treated, the PD-1 antigen binding protein or antigen binding fragment is a PD-1 antagonist that blocks binding of human PD-L1 to human PD-1, or blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

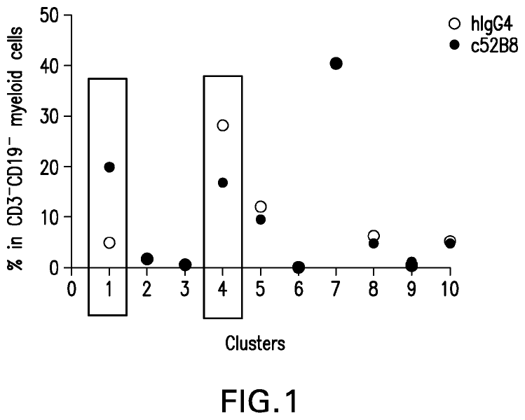

Examples of mAbs that bind to human PD-1, useful in the treatment methods and uses of the invention, are described in U.S. Pat. Nos. 7,521,051, 8,008,449, and 8,354,509. Specific anti-human PD-1 mAbs useful as a PD-1 antagonist in the treatment methods, compositions, and uses of the present invention include: pembrolizumab (formerly known as MK-3475, SCH 900475 and lambrolizumab), a humanized IgG4 mAb with the structure described in WHO Drug Information, Vol. 27, No. 2, pages 161-162 (2013) and which comprises the heavy and light chain amino acid sequences shown in FIG. 1, and the humanized antibodies h409A11, h409A16 and h409A17, which are described in WO 2008/156712 and in Table 6.

In some embodiments of the treatment methods, compositions, kits and uses of the present invention, the anti-PD-1 antigen binding protein, antibody, or antigen binding fragment, comprises: (a) light chain CDRs comprising a sequence of amino acids as set forth in SEQ ID NOs: 1, 2 and 3 and heavy chain CDRs comprising a sequence of amino acids as set forth in SEQ ID NOs: 6, 7 and 8; or (b) light chain CDRs comprising a sequence of amino acids as set forth in SEQ ID NOs: 11, 12 and 13 and heavy chain CDRs comprising a sequence of amino acids as set forth in SEQ ID NOs: 14, 15 and 16. In some embodiments, the anti-PD-1 antigen binding protein, antibody or antigen binding fragment is a human antibody. In other embodiments, the anti-PD-1 antigen binding protein, antibody or antigen binding fragment is a humanized antibody. In other embodiments, the anti-PD-1 antigen binding protein, antibody or antigen binding fragment is a chimeric antibody. In specific embodiments, the anti-PD-1 antigen binding protein, antibody or antigen binding fragment is a monoclonal antibody.

In other embodiments of the treatment methods, compositions, and uses of the present invention, the anti-PD-1 antigen binding protein, antibody, or antigen binding fragment, specifically binds to human PD-1 and comprises (a) a heavy chain variable region comprising an amino acid sequence as set forth in SEQ ID NO: 24, or a variant thereof, and (b) a light chain variable region comprising an amino acid sequence selected from the group consisting of SEQ ID NO: 25 or a variant thereof; SEQ ID NO: 26 or a variant thereof; and SEQ ID NO: 27 or a variant thereof.

In another embodiment of the treatment methods, compositions, and uses of the present invention, the anti-PD-1 antigen binding protein or antigen binding fragment is a monoclonal antibody which specifically binds to human PD-1 and comprises (a) a heavy chain comprising or consisting of a sequence of amino acids as set forth in SEQ ID NO: 28, or a variant thereof, and (b) a light chain comprising or consisting of a sequence of amino acids as set forth in SEQ ID NO: 29, or a variant thereof; SEQ ID NO: 30, or a variant thereof, or SEQ ID NO: 31, or a variant thereof.

In yet another embodiment of the treatment methods, compositions and uses of the invention, the anti-PD-1 antigen binding protein or antigen binding fragment is a monoclonal antibody which specifically binds to human PD-1 and comprises (a) a heavy chain comprising or consisting of a sequence of amino acids as set forth in SEQ ID NO: 28 and (b) a light chain comprising or consisting of a sequence of amino acids as set forth in SEQ ID NO: 29.

Table 6 and Table 7 below provides a list of the amino acid sequences of exemplary anti-PD-1 mAbs for use in the treatment methods, compositions, kits and uses of the present invention.

TABLE 6
Exemplary anti-human PD-1 antibodies
A. Comprises light and heavy chain CDRs of hPD-1.09A in WO2008/156712 (light
and heavy chain CDRs of pembrolizumab)

| | |
|---|---|
| CDRL1 | RASKGVSTSGYSYLH |
| | SEQ ID NO: 224 |
| CDRL2 | LASYLES |
| | SEQ ID NO: 225 |
| CDRL3 | QHSRDLPLT |
| | SEQ ID NO: 226 |
| CDRH1 | NYYMY |
| | SEQ ID NO: 227 |
| CDRH2 | GINPSNGGTNFNEKFKN |
| | SEQ ID NO: 228 |
| CDRH3 | RDYRFDMGFDY |
| | SEQ ID NO: 229 |

B. Comprises light and heavy chain CDRs of hPD-1.08A in WO2008/156712

| | |
|---|---|
| CDRL1 | RASKSVSTSGFSYLH |
| | SEQ ID NO: 230 |
| CDRL2 | LASNLES |
| | SEQ ID NO: 231 |
| CDRL3 | QHSWELPLT |
| | SEQ ID NO: 232 |
| CDRH1 | SYYLY |
| | SEQ ID NO: 233 |
| CDRH2 | GVNPSNGGTNFSEKFKS |
| | SEQ ID NO: 234 |
| CDRH3 | RDSNYDGGFDY |
| | SEQ ID NO: 235 |

C. Comprises the mature h109A heavy chain variable (VH) region and one of the
mature K09A light chain variable (VL) regions in WO 2008/156712

| | |
|---|---|
| Heavy chain VH | QVQLVQSGVEVKKPGASVKVSCKASGYTFTNYYMYWVRQ |
| | APGQGLEWMGGINPSNGGTNFNEKFKNRVTLTTDSSTTTAY |
| | MELKSLQFDDTAVYYCARRDYRFDMGFDYWGQGTTVTVS |
| | S |
| | SEQ ID NO: 236 (VH of pembrolizumab) |
| Light chain VL | EIVLTQSPATLSLSPGERATLSCRASKGVSTSGYSYLHWYQQ |
| | KPGQAPRLLIYLASYLESGVPARFSGSGSGTDFTLTISSLEPE |
| | DFAVYYCQHSRDLPLTFGGGTKVEIK |
| | SEQ ID NO: 237 (VL of pembrolizumab) or |
| | EIVLTQSPLSLPVTPGEPASISCRASKGVSTSGYSYLHWYLQK |
| | PGQSPQLLIYLASYLESGVPDRFSGSGSGTDFTLKISRVEAED |
| | VGVYYCQHSRDLPLTFGQGTKLEIK |
| | (SEQ ID NO: 238) or |
| | DIVMTQTPLSLPVTPGEPASISCRASKGVSTSGYSYLHWYLQ |
| | KPGQSPQLLIYLASYLESGVPDRFSGSGSGTAFTLKISRVEAE |
| | DVGLYYCQHSRDLPLTFGQGTKLEIK |
| | SEQ ID NO: 239 |
| Heavy chain | QVQLVQSGVEVKKPGASVKVSCKASGYTFTNYYMYWVRQ |
| | APGQGLEWMGGINPSNGGTNFNEKFKNRVTLTTDSSTTTAY |
| | MELKSLQFDDTAVYYCARRDYRFDMGFDYWGQGTTVTVS |
| | SASTKGPSVFPLAPCSRSTSESTAALGCLVKDYFPEPVTVSW |
| | NSGALTSGVHTFPAVLQSSGLYSLSSVVTVPSSSLGTKTYTC |
| | NVDHKPSNTKVDKRVESKYGPPCPPCPAPEFLGGPSVFLFPP |
| | KPKDTLMISRTPEVTCVVVDVSQEDPEVQFNWYVDGVEVH |
| | NAKTKPREEQFNSTYRVVSVLTVLHQDWLNGKEYKCKVSN |
| | KGLPSSIEKTISKAKGQPREPQVYTLPPSQEEMTKNQVSLTCL |
| | VKGFYPSDIAVEWESNGQPENNYKTTPPVLDSDGSFFLYSRL |
| | TVDKSRWQEGNVFSCSVMHEALHNHYTQKSLSLSLGK |
| | SEQ ID NO: 240 (heavy chain of pembrolizumab) |
| Light chain | EIVLTQSPATLSLSPGERATLSCRASKGVSTSGYSYLHWYQQ |
| | KPGQAPRLLIYLASYLESGVPARFSGSGSGTDFTLTISSLEPE |
| | DFAVYYCQHSRDLPLTFGGGTKVEIKRTVAAPSVFIFPPSDE |
| | QLKSGTASVVCLLNNFYPREAKVQWKVDNALQSGNSQESV |
| | TEQDSKDSTYSLSSTLTLSKADYEKHKVYACEVTHQGLSSP |
| | VTKSFNRGEC |
| | SEQ ID NO: 241 (light chain of pembrolizumab) or |
| | EIVLTQSPLSLPVTPGEPASISCRASKGVSTSGYSYLHWYLQK |
| | PGQSPQLLIYLASYLESGVPDRFSGSGSGTDFTLKISRVEAED |
| | VGVYYCQHSRDLPLTFGQGTKLEIKRTVAAPSVFIFPPSDEQ |
| | LKSGTASVVCLLNNFYPREAKVQWKVDNALQSGNSQESVT |
| | EQDSKDSTYSLSSTLTLSKADYEKHKVYACEVTHQGLSSPV |
| | TKSFNRGEC |
| | SEQ ID NO: 242 or |
| | DIVMTQTPLSLPVTPGEPASISCRASKGVSTSGYSYLHWYLQ |
| | KPGQSPQLLIYLASYLESGVPDRFSGSGSGTAFTLKISRVEAE |
| | DVGLYYCQHSRDLPLTFGQGTKLEIKRTVAAPSVFIFPPSDE |
| | QLKSGTASVVCLLNNFYPREAKVQWKVDNALQSGNSQESV |
| | TEQDSKDSTYSLSSTLTLSKADYEKHKVYACEVTHQGLSSP |
| | VTKSFNRGEC |
| | SEQ ID NO: 243 |

TABLE 7
Nivolumab Heavy and Light Chains
Nivolumab Light Chain

| | |
|---|---|
| CDR1 | RASQSVSSYLA |
| | (SEQ ID NO: 244) |
| CDR2 | DASNRAT |
| | (SEQ ID NO: 245) |
| CDR3 | QQSSNWPRT |
| | (SEQ ID NO: 246) |
| Variable | EIVLTQSPATLSLSPGERATLSCRASQSVSSYLAWYQQKPGQAPRLL |
| Region | IYDASNRATGIPARFSGSGSGTDFTLTISSLEPEDFAVYYCQQSSNWP |
| | RTFGQGTKVEIK |
| | (SEQ ID NO: 247) |
| Light | EIVLTQSPATLSLSPGERATLSCRASQSVSSYLAWYQQKPGQAPRLLI |
| Chain | YDASNRATGIPARFSGSGSGTDFTLTISSLEPEDFAVYYCQQSSNWPR |
| | TFGQGTKVEIKRTVAAPSVFIFPPSDEQLKSGTASVVCLLNNFYPREA |
| | KVQWKVDNALQSGNSQESVTEQDSKDSTYSLSSTLTLSKADYEKHK |
| | VYACEVTHQGLSSPVTKSFNRGEC |
| | (SEQ ID NO: 248) |

Nivolumab Heavy Chain

| | |
|---|---|
| CDR1 | NSGMH |
| | (SEQ ID NO: 249) |
| CDR2 | VIWYDGSKRYYADSVKG |
| | (SEQ ID NO: 250) |
| CDR3 | NDDY |
| | (SEQ ID NO: 251) |
| Variable | QVQLVESGGGVVQPGRSLRLDCKASGITFSNSGMHWVRQAPGKGLE |
| Region | WVAVIWYDGSKRYYADSVKGRFTISRDNSKNTLFLQMNSLRAEDTA |
| | VYYCATNDDYWGQGTLVTVSS |
| | (SEQ ID NO: 252) |
| Heavy | QVQLVESGGGVVQPGRSLRLDCKASGITFSNSGMHWVRQAPGKGLE |
| Chain | WVAVIWYDGSKRYYADSVKGRFTISRDNSKNTLFLQMNSLRAEDTA |
| | VYYCATNDDYWGQGTLVTVSSASTKGPSVFPLAPCSRSTSESTAALG |
| | CLVKDYFPEPVTVSWNSGALTSGVHTFPAVLQSSGLYSLSSVVTVPS |
| | SSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHN |
| | AKTKPREEQFNSTYRVVSVLTVLHQDWLNGKEYKCKVSNKGLPSSI |
| | EKTISKAKGQPREPQVYTLPPSQEEMTKNQVSLTCLVKGFYPSDIAVE |
| | WESNGQPENNYKTTPPVLDSDGSFFLYSRLTVDKSRWQEGNVFSCS |
| | VMHEALHNHYTQKSLSLSLGK |
| | (SEQ ID NO: 253) |

The anti-ILT3 antigen binding proteins or antigen binding fragments herein may be used alone or in combination with other therapies. For example, the combination therapy may include a composition comprising an anti-ILT3 antigen binding protein, antibody or antigen binding fragment co-formulated with, and/or co-administered with, one or more additional therapeutic agents. e.g., one or more anti-cancer agents, cytotoxic or cytostatic agents, hormone treatment, vaccines, chemotherapy, and/or other immunotherapies. In other embodiments, the anti-ILT3 antigen binding protein, antibody or antigen binding fragment is administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The anti-ILT3 antigen binding protein, antibody or antigen binding fragment may be administered concurrently with, prior to, or subsequent to, one or more other additional therapies or therapeutic agents. The anti-ILT3 antigen binding protein, antibody or antigen binding fragment and the other agent or therapeutic protocol may be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In certain embodiments, an anti-ILT3 antigen binding protein or antigen binding fragment described herein is administered in combination with one or more check point inhibitors or antagonists of programmed death receptor 1 (PD-1) or its ligand PD-L1 and PD-L2. The inhibitor or antagonist may be an antigen binding protein, an antibody, an antigen binding fragment, an immunoadhesin, a fusion protein, or oligopeptide. In some embodiments, the anti-PD-1 antibody is chosen from nivolumab (OPDIVO®, Bristol Myers Squibb, New York, New York), pembrolizumab (KEYTRUDA®, Merck Sharp & Dohme Corp, Kenilworth, NJ USA), cetiplimab (Regeneron, Tarrytown, NY) or pidilizumab (CT-011). In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 inhibitor is AMP-224. In some embodiments, the PD-L1 inhibitor is anti-PD-L1 antibody such durvalumab (IMFINZIR, AstraZeneca, Wilmington, DE), atezolizumab (TECENTRIQ®. Roche, Zurich, CH), or avelumab (BAVENCIO®, EMD Serono, Billerica, MA). In some embodiments, the anti-PD-L1 binding antagonist is chosen from YW243.55.S70. MPDL3280A. MEDI-4736. MSB-0010718C. or MDX-1105.

MDX-1105, also known as BMS-936559, is an anti-PD-L1 antibody described in WO2007/005874. Antibody YW243.55.S70 is an anti-PD-L1 described in WO 2010/077634 (heavy and light chain variable region sequences shown in SEQ ID NOs. 20 and 21, respectively).

Nivolumab, also known as OPDIVO®, MDX-1106-04, ONO-4538, or BMS-936558, is a fully human IgG4 anti-PD-1 antibody described in WO2006/121168 and U.S. Pat. No. 8,008,449.

Pembrolizumab, also known as KEYTRUDA®, lambrolizumab, MK-3475 or SCH-900475, is a humanized anti-PD-1 antibody described in U.S. Pat. No. 8,354,509 and WO2009/114335 and disclosed, e.g., in Hamid, et al., New England J. Med. 369 (2): 134-144 (2013). The heavy and light chains for pembrolizumab are shown by the amino acid sequences set forth in SEQ ID Nos: 225 and 226, respectively.

Pidilizumab, also known as CT-011 (Cure Tech) is a humanized IgG1 monoclonal antibody that binds to PD-1. Pidilizumab and other humanized anti-PD-1 monoclonal antibodies are disclosed in WO2009/101611. Other anti-PD-1 antibodies include AMP 514 (Amplimmune), among others, e.g., anti-PD-1 antibodies disclosed in U.S. Pat. No. 8,609,089; U.S Publication No. 2010028330; and U.S Publication No. 20120114649.

AMP-514 (MEDI0680; MedImmune LLC, Gaithersburg, MD) is a monoclonal antibody that binds PD-1.

PDR001 (spartalizumab; Novartis) is a monoclonal antibody that binds PD-1 and is disclosed in U.S. Pat. No. 9,683,048.

BGB-A317 (tislelizumab; Beigene) is a monoclonal antibody that binds PD-1 and is disclosed in U.S. Pat. No. 8,735,553.

MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No. 20120039906.

MGA012 (MacroGenics, Rockville, MD) a monoclonal antibody that binds PD-1.

AMP-224 (B7-DCIg; Amplimmune; e.g., disclosed in WO2010/027827 and WO2011/066342), is a PD-L2 Fc fusion soluble receptor that blocks the interaction between PD-1 and B7-H1.

Other anti-PD-L1 binding agents include YW243.55.S70 (heavy and light chain variable regions are shown in SEQ ID NOs 20 and 21 in WO2010/077634) and MDX-1105 (also referred to as BMS-936559). It and other anti-PD-L1 binding agents are disclosed in WO2007/005874).

In some embodiments, the ILT3 antigen binding proteins or antigen binding fragments herein and the PD-1 or PD-L1 antagonist may be used in combination with one or more additional therapeutic agents, e.g., one or more anti-cancer agents, cytotoxic or cytostatic agents, hormone treatment, vaccines, chemotherapy, and/or other immunotherapies. In other embodiments, the anti-ILT3 antigen binding protein, antibody or antigen binding fragment is administered in combination with other therapeutic treatment modalities, including surgery, radiation, cryosurgery, and/or thermotherapy.

### Dosing and Administration

Provided herein are dosing regimens and routes of administration for treating cancer and in specific embodiments, CMML using an anti-ILT3 antigen binding protein or antigen binding fragment (e.g. any of the mAbs in Table 4), or a combination of an anti-ILT3 antigen binding protein or antigen binding fragment (e.g. any of the mAbs in Table 4).

The anti-ILT3 antigen binding protein or antigen binding fragment and the anti-PD1 antigen binding protein or antigen binding fragment disclosed herein may be administered by continuous infusion, or by doses administered, e.g., daily, 1-7 times per week, weekly, bi-weekly, tri-weekly, every four weeks, every five weeks, every 6 weeks, monthly, bimonthly, quarterly, semiannually, annually, etc., either concurrently or consecutively. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. A total dose for a treatment interval is generally at least 0.05 μg/kg body weight, more generally at least 0.2 g/kg, 0.5 g/kg, 1 g/kg, 10 g/kg, 100 g/kg, 0.25 mg/kg, 1.0 mg/kg, 2.0 mg/kg, 5.0 mg/ml, 10 mg/kg, 25 mg/kg, 50 mg/kg or more. Doses may also be provided to achieve a pre-determined target concentration of the antigen binding protein (e.g., anti-ILT3 antibody) or antigen binding fragment in the subject's serum, such as 0.1, 0.3, 1, 3, 10, 30, 100, 300 μg/mL or more. In some embodiments, the anti-ILT3 antigen binding protein or antigen binding fragment is administered intravenously, on a weekly, biweekly, triweekly, every 3 weeks, every 4 weeks, every 5 weeks, every 6 weeks, monthly, bimonthly, or quarterly basis at 10, 20, 50, 80, 100, 200, 300, 400, 500, 1000 or 2500 mg/subject.

In some embodiments, the anti-ILT3 antigen binding protein or antigen binding fragment is administered intravenously, on a weekly, biweekly, triweekly, every 4 weeks, every 5 weeks, every 6 weeks, monthly, bimonthly, or quarterly basis at 10, 20, 50, 80, 100, 200, 500, 1000 or 2500 mg/subject. In some specific methods, the dose of the anti-ILT3 antigen binding protein or antigen binding fragment is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of an anti-ILT3 antigen binding protein or antigen binding fragment may be between about 0.2 mg and about 2 mg. In some specific methods, the dose of an anti-ILT3 antigen binding protein or antigen binding fragment may be between 0.2 mg and 2 mg. In some specific methods, the dose of an anti-ILT3 antigen binding protein or antigen binding fragment may be between about 0.2 mg and about 2250 mg. In some specific methods, the dose of an anti-ILT3 antigen binding protein or antigen binding fragment may be between about 0.2 mg and about 750 mg. In some specific methods, the dose of an anti-ILT3 antigen binding protein or antigen binding fragment may be between 0.2 mg and 2250 mg. In some specific methods, the dose of an anti-ILT3 antigen binding protein or antigen binding fragment may be between 0.2 mg and 750 mg. In some specific methods, the dose of an anti-ILT3 antigen binding protein or antigen binding fragment may be between about 7.5 mg and about 2250 mg. In some specific methods, the dose of an anti-ILT3 antigen binding protein or antigen binding fragment may be between about 7.5 mg and about 750 mg. In some specific methods, the dose of an anti-ILT3 antigen binding protein or antigen binding fragment may be between 7.5 mg and 2250 mg. In some specific methods, the dose of an anti-ILT3 antigen binding protein or antigen binding fragment may be between 7.5 mg and 750 mg. In some specific methods, the dose of an anti-ILT3 antigen binding protein or antigen binding fragment may be between about 25 mg and about 750 mg. In some specific methods, the dose of an anti-ILT3 antigen binding protein or antigen binding fragment may be between 25 mg and 750 mg. In some specific methods, the dose of an anti-ILT3 antigen binding protein or antigen binding fragment may be between about 75 mg and about 750 mg. In some specific methods, the dose of an anti-ILT3 antigen binding protein or antigen binding fragment may be between 75 mg and 750 mg. In some specific methods, the dose of an anti-ILT3 antigen binding protein or antigen binding fragment may be between about 225 mg and about 750 mg. In some specific methods, the dose of an anti-ILT3 antigen binding protein or antigen binding fragment may be between 225 mg and 750 mg. In some specific methods, the dose of an anti-ILT3 antigen binding protein or antigen binding fragment may be about 0.2 mg, about 0.7 mg, or about 2 mg. In some specific methods, the dose of an anti-ILT3 antigen binding protein or antigen binding fragment may be about 7.5 mg, about 25 mg, about 75 mg, about 225 mg, about 750 mg, or about 2250 mg. In some specific methods, the dose of an anti-ILT3 antigen binding protein or antigen binding fragment may be 0.2 mg, 0.7 mg, or 2 mg. In some specific methods, the dose of an anti-ILT3 antigen binding protein or antigen binding fragment may be 7.5 mg, 25 mg, 75 mg, 225 mg, 750 mg, or 2250 mg. In some specific methods, the dose of an anti-ILT3 antigen binding protein or antigen binding fragment may be about 750 mg. In some specific methods, the dose of an anti-ILT3 antigen binding protein or antigen binding fragment may be 750 mg.

### General Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA). Standard methods also appear in Ausubel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Shepherd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies or antigen binding fragments can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Standard methods of histology of the immune system are described (see, e.g., Muller-Hanmelink (ed.) (1986) Human Thymus: Histopathology and Pathology, Springer Verlag, New York, NY; Hiatt, et al. (2000) Color Atlas ofHistology, Lippincott, Williams, and Wilkins, Phila, PA; Louis, et al. (2002) Basic Histology: Text and Atlas, McGraw-Hill, New York, NY). Software packages and databases for determining, e.g., antigenic fragments, leader sequences, protein folding, functional domains, glycosylation sites, and sequence alignments, are available (see, e.g., GenBank, VECTOR NTI Suite (Informax, Inc, Bethesda, MD); GCG Wisconsin Package (Accelrys, Inc., San Diego, CA); DECYPHER (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

### Example 1: Effect of Anti-ILT3 Parental Antibody 52B8 on AML Patient PBMC

The effect of anti-ILT3 parental antibody 52B8 on AML patient PBMC was assessed in vitro. AML patient PBMC (761L) with high ILT3 expression on myeloid cells was treated with 52B8 or with human IgG4 (hIgG4). AML PBMC were treated with 52B8 or hIgG4 isotype control (1 mg/ml) for 24 hours in vitro. Treated PBMC were stained with Abs (see Table 8 below, listing the staining panel and antibody sources; Fluidigm, South San Francisco, CA, USA; Invitrogen, Waltham, MA, USA; eBioscience, Waltham, MA, USA; R&D Systems, Minneapolis, MN, USA) and profiled and quantitated using cytometry by time of flight (CyTOF) to detect PBMC phenotypes. Table 9 below lists the CyTOF phenotype of myeloid cell clusters 1 & 4 in AML PBMC.

TABLE 8
Staining panel for CyTOF analysis

| Antibody | metal-tag | Cat.# |
|---|---|---|
| CD3 | 154Sm | 3154003B (Fluidigm) |
| CD4 | 145Nd | 3145001B (Fluidigm) |
| CD7 | 153Eu | 3153014B (Fluidigm) |
| CD8 | 162Dy | 3162015B (Fluidigm) |
| CD11b | 209Bi | 3209003B (Fluidigm) |
| CD13 | 160Gd | 3160014B (Fluidigm) |
| CD14 | 175Lu | 3175015B (Fluidigm) |
| CD19 | 142Nd | 3142001B (Fluidigm) |
| CD20 | 147Sm | 3147001B (Fluidigm) |
| CD25 | 169Tm | 3169003B (Fluidigm) |
| CD33 | 163Dy | 3163023B (Fluidigm) |
| CD34 | 148Nd | 3148001B (Fluidigm) |
| CD38 | 167Er | 3167001B (Fluidigm) |
| CD44 | 166Er | 3166001B (Fluidigm) |
| CD45 | 89Y | 3089003B (Fluidigm) |
| CD56 | 155Gd | 3155008B (Fluidigm) |
| CD64 | 146Nd | 3146006B (Fluidigm) |
| CD69 | 144Nd | 3144018B (Fluidigm) |
| CD86 | 150Nd | 3150020B (Fluidigm) |
| CD117 | 143Nd | 3143001B (Fluidigm) |
| CD123 | 151Eu | 3151001B (Fluidigm) |
| CD127 | 149Sm | 3149011B (Fluidigm) |
| CD135(FLT3) | 158Gd | 3158019B (Fluidigm) |
| CD184(CXCR4) | 156Gd | 3156029B (Fluidigm) |
| TCR Vd2 | 152Sm | 331402 (Biolegend) |
| TCR Vd1 | 168Er | TCR1730 (Invitrogen) |
| HLA-DR | 170Er | 3154003B (Fluidigm) |
| TCRab | 176Yb | 3176015B (Fluidigm) |
| CD274(PD-L1) | 159Tb | 3159029B (Fluidigm) |
| CD279(PD-1) | 174Yb | 3174020B (Fluidigm) |
| PI-16 | 141Pr | in-house |
| ILT3(CD85k) | 173Yb | 16-5139-85 (eBioscience) |
| ILT4 | 164Dy | MAB2078 (R&D Systems) |
| IFNg | 165Ho | 3165002B (Fluidigm) |
| Ki67 | 161Dy | 3161007B (Fluidigm) |
| GranzymeB | 171Yb | 3171002B(Fluidigm) |
| Myeloperoxidase (2C7) | 172Yb | MA1-80878 (Invitrogen) |

TABLE 9
CyTOF phenotype of myeloid cell clusters 1 & 4 in AML PBMC

| Cluster/ | | | | | | | | HLA- |
|---|---|---|---|---|---|---|---|---|
| Intensity | CD11b | CD13 | CD4 | CD33 | CD14 | CD64 | CD34 | DR |
| 1 | 606 | 762 | 43 | 101 | 12 | 204 | 15 | 139 |
| 4 | 523 | 365 | 34 | 40 | 4 | 148 | 1 | 158 |

Cluster 1 (monocytic myeloid cells):
CD11b⁺CD13⁺CD4⁺CD33⁺CD34⁺CD14⁺CD64⁺HLA⁻DR⁺
Cluster 4 (tumor blasts):
CD11b⁺CD13⁺CD4⁺CD33⁺CD34⁻CD14⁻CD64^{low}HLA⁻DR⁺

FIG. 1 shows a dot plot quantitating and comparing the percentage of 10 clusters of myeloid cells between the 52B8 and hIgG4 isotype treatments. As shown in FIG. 1, treatment of AML PBMCs with 52B8 mAb (filled circles) decreased the frequency of tumor blasts (cluster 4) and increased the monocytic myeloid population (cluster 1, filled circles).

### Example 2: Anti-ILT3 Antibody Inhibits Growth of AML Cells In Vivo

The antitumor efficacy of anti-ILT3 parental antibody 52B8 as a single agent was assessed in the systemic MV-4-11 myelomonocytic leukemia model in humanized mouse. NOD. Cg-Prkdcscid Il2rgtm1 Wjl/SzJ (NSG™) mice were inoculated with human PBMC (10⁶/mouse) and MV-4-11 luc cells (10⁶/mouse) by IV injection.

For generation of MV-4-11 luc cells, a live luciferase reporter virus was generated using Clontech GP2-293 packaging cells (Takara Bio, Mountain View, CA, USA), transfected with pLXSN-Luc and pVSV-G vectors using FuGENE HD Transfection Reagent (Roche, Mannheim, Germany). MV-4-11 cells were infected with the luciferase reporter virus and luciferase-positive cells selected with Geneticin selection antibiotic (G418) (Invitrogen, Carlsbad, CA, USA). Luciferase activity was checked in vitro using bioluminescent imaging (BLI). Cells were cryopreserved in liquid nitrogen using cell culture freezing media prior to culturing for inoculation.

For assessment of 52B8 efficacy, animals were assigned to two treatment groups at 10 mice per group one week after the cell inoculation. 52B8 or a hIgG4 isotype control was administered IP at 10 mg/kg on days 7, 14, 21, 28, and 35. MV-4-11 luc cell growth in vivo was measured by BLI using the IVIS® Spectrum In Vivo Imaging System (Perkin Elmer, Waltham, MA, USA). Measurements were taken weekly for the first 4 weeks after inoculation, then twice weekly. Statistical analysis between the two groups was performed with two-way ANOVA with the Geisser-Greenhouse correction. Post hoc analysis was done with a Sidak's Multiple Comparison Test. **: p<0.01; *: p<0.05. Terminal bone marrow (BM) samples from the treated groups were profiled by CyTOF. MV-4-11 cells were identified by human CD3⁻CD19⁻CD45⁺.

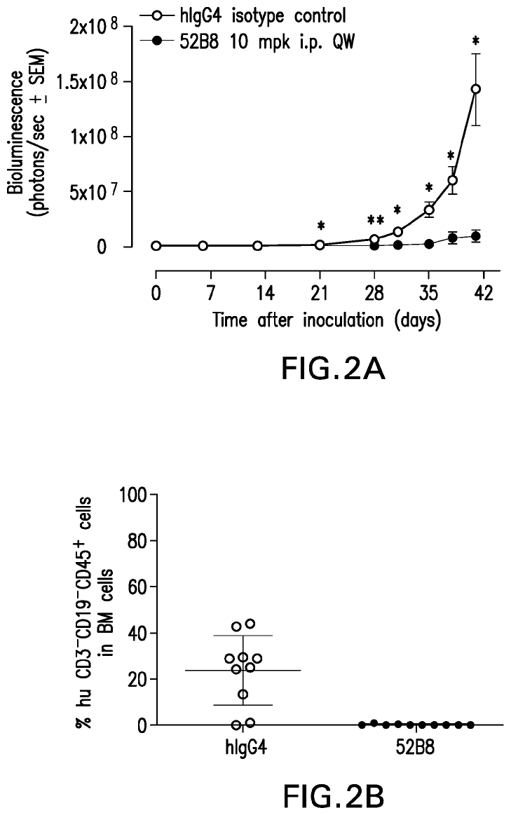

As seen in FIG. 2A, mice receiving hIgG4 isotype control antibody starting on day post engraftment of the tumor cells showed a statistically significant increase in MV-4-11 luc cell growth. However, mice receiving 52B8 treatment starting on day 7 post engraftment of the tumor cells diminished MV-4-11 growth in vivo. FIG. 2B shows a dot plot of the percentage of MV-4-11 luc cells as a percentage of bone marrow cells from each of the treated groups. BM samples from mice treated with 52B8 starting on day 7 post-engraftment of the tumor cells showed no MV-4-11 luc cells, while BM samples from mice treated with hIgG4 showed large percentages of MV-4-11 luc cells.

### Example 3: Anti-ILT3 Antibody and IFNγ Production by Donor T Cells

The ability of anti-ILT3 antibody to affect IFN-gamma production by T cells was examined. Anti ILT-3 antibody c52B8 or control human IgG4 antibody (hIgG4) was incubated with co-cultures of human CD8+ T cells from different human donors and irradiated THP-1 cells (human monocyte cell line from an acute monocytic leukemia patient) at a T cell:THP-1 ratio of 8:1. Control hIgG4 antibody was incubated at a concentration of 10 μg/mL, and 52B8 mAb was incubated at 10, 1, and 0.1 μg/mL. The incubated co-cultures were then stimulated with anti-CD3/CD28 coated beads. Cell culture supernatants were then assayed for IFN-γ expression using a V-PLEX human IFN-γ assay kit (Mesoscale Discovery, Rockville, MD, USA).

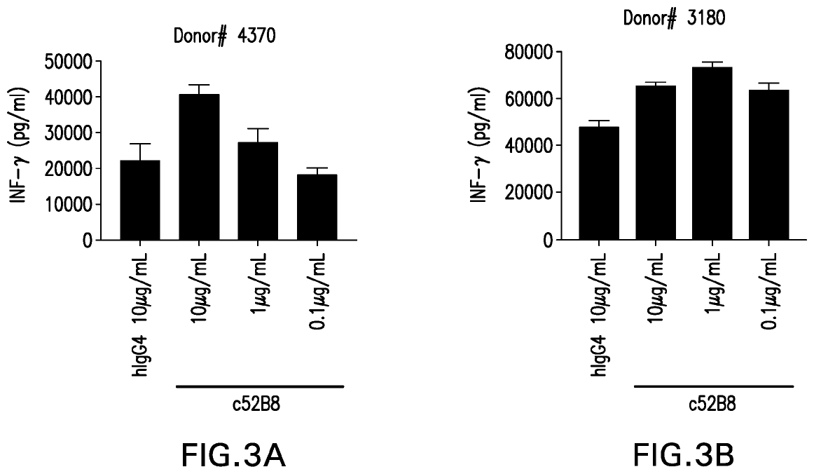

FIGS. 3A and 3B each show bar graphs of IFN-γ expression in CD8+ T cells from two different donors. The anti-ILT3 antibody 52B8 greatly enhanced the production of pro-inflammatory cytokine IFN-γ, with 10 μg/mL of 52B8 causing an increase in IFN-γ well above that of control antibody.

The following examples using mAb number 46 as a representative anti-ILT3 antibody are meant to be illustrative and should not be construed as further limiting. The contents of the figures and all references, patents, and published patent applications cited throughout this application are expressly incorporated herein by reference.

### Example 4: Rationale for Treating Patients with CMML

CMML is a myeloid neoplasm characterized by dysplasia, abnormal production and accumulation of monocytic cells with ˜30% to 40% risk of transforming into AML. Expression of ILT3 and role in CMML and MDS are unknown. However, Chien et al. [Chien, K. S., et al. 2020] recently investigated LILRB4 expression in 19 CMML, 27 MDS-CMML patients, and a few healthy volunteers. The study team found that LILRB4 RNA expression was increased in CMML patients when compared with MDS patients and healthy controls and slightly increased in patients who responded to hypomethylating agents. This study also showed that interferon gamma response and CTLA4 signaling genes positively correlated with LILRB4 expression in CMML patients. Given that there are no effective treatments available for CMML patients with refractory or relapsed disease, the inventors decided to explore the clinical effects of an anti-ILT3 mAb in this myeloid malignancy.

### Example 5: Phase 1b Study to Evaluate Anti-ILT3 Antibody for Relapsed/Refractory AML and CMML

### Study Design

This is a multicenter, open-label, Phase 1b study to evaluate safety, tolerability, PK and pharmacodynamics of anti-ILT3 antibody in participants with relapsed/refractory AML. The study will enroll participants with AML subtypes of acute myelomonocytic leukemia or acute monoblastic/monocytic leukemia per 2016 WHO classification [Arber, D. A., et al. 2016] or CMML.

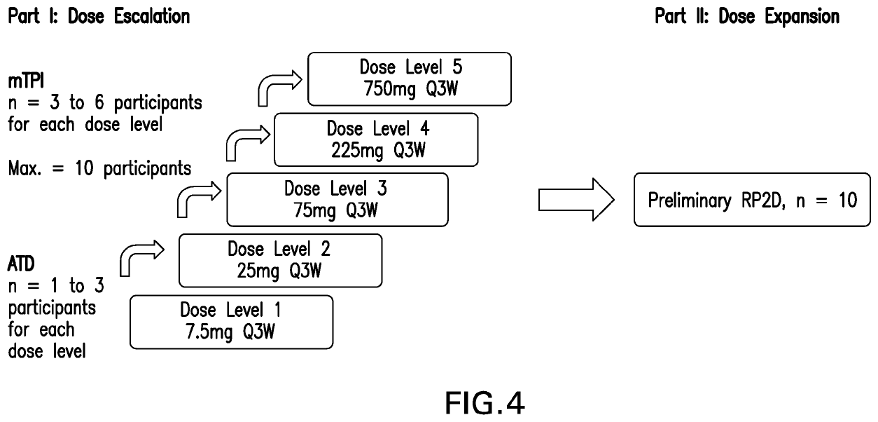

There are 2 parts in this study: Dose Escalation (Part 1) and Dose Expansion (Part 2). For Part 1, initial dose escalation will follow an accelerated titration design (ATD) to evaluate 2 low dose levels (DL): DL1 of 7.5 mg and DL2 of 25 mg, with each group enrolling 1 to 3 participants. Once the study passes DL2, further dose escalation will follow the mTPI design [Ji, Y. et al. 2013] to evaluate dose levels of 75 mg, 225 mg, and 750 mg anti-ILT3 antibody, respectively, in accordance with dose levels evaluated in the solid tumor study. During this study, a higher dose level up to 2250 mg may be explored depending on the combined safety, PK, and pharmacodynamics data available. Each dose level under mTPI will enroll 3 to 6 participants initially (with at least 2 participants with AML) with potential expansion to a maximum of 10 participants (with at least 5 participants with AML). FIG. 4 shows a schematic drawing of the study design. Intermediate or higher dose levels may be evaluated. The maximum treatment duration is 35 cycles (approximately 24 months). Intraparticipant dose escalation is allowed for participants enrolled to ATD dose levels up to 75 mg per dose.

Progression from one DL to the next higher DL is based on the evaluation of DLT. The ATD cohort will end early if a Grade 2 or higher treatment-related toxicity occurs. In that situation, the dose level will be evaluated per mTPI. During dose escalation, a higher dose level cannot be initiated until the previous lower dose level has cleared DLT.

Dose finding in Part 1 will end after 10 participants have been treated at any dose level. The pool-adjacent-violators algorithm [Ji, Y. et al. 2013] will be used to estimate the DLT rates across doses in each treatment arm under the assumption of monotonicity between DLT rates and dose levels. The totality of the data including safety events that occur within or beyond the DLT window, tolerability, preliminary antitumor activity, PK, and pharmacodynamics across all the dose levels will be considered before deciding a preliminary RP2D for carrying forward to Part 2. Approximately 20 participants will be enrolled in Part 1.

Once a preliminary RP2D is identified in Part 1, approximately 10-15 additional participants with relapsed/refractory AML will be enrolled at the RP2D for Part 2. The study will enroll approximately 30-35 participants in total.

Study will include a screening period of maximum of 21 days. Eligible participants will receive study treatment and be monitored carefully via physical examinations and laboratory tests for safety. AEs will be evaluated by the investigator per NCI CTCAE 5.0.

Clinical activities will be evaluated for the changes in AML blasts in bone marrow as well as in peripheral blood in accordance with ELN 2017 response criteria listed below in Table 10. Clinical activities for CMML will be evaluated per 2015 Intemational Consortium Proposal of Uniform Response Criteria for MDS/MPN in Adults (see Table 11)

TABLE 10
ELN 2017 response criteria

| | | |
|---|---|---|
| Category | Definition | Comment |

Response

| | | |
|---|---|---|
| Complete remission (CR) | Bone marrow blasts <5%; | MRD⁺ or unknown |
| | absence of circulating | |
| | blasts and blasts with Auer | |
| | rods; absence of | |
| | extramedullary disease; | |
| | ANC ≥1.0 × 10⁹/L | |
| | (1000/μL); platelet | |
| | count ≥100 × 10⁹/L | |
| | (100,000/L) | |
| CR with incomplete | All CR criteria except for | |
| hematologic recovery | residual neutropenia | |
| (CRᵢ) | (<1.0 × 10⁹/L [1000/μL]) or | |
| | thrombocytopenia (<100 × | |
| | 10⁹/L [100,000/μL]) | |
| Morphologic leukemia- | Bone marrow blasts <5%; | Marrow should not merely |
| free state (MLFS) | absence of blasts with | be “aplastic”; at least 200 |
| | Auer rods; absence of | cells should be enumerated |
| | extramedullary disease; no | or cellularity should be at |
| | hematologic recovery | least 10% |
| | required | |
| Partial remission (PR) | All hematologic criteria of | Especially important in the |
| | CR; decrease of bone | context of Phase 1-2 |
| | marrow blast percentage to | clinical trials |
| | 5% to 25%; and decrease | |
| | of pre-treatment bone | |
| | marrow blast percentage | |
| | by at least 50% | |

Treatment failure

| | | |
|---|---|---|
| Primary Refractory | No CR or CRᵢ after 2 | Regimens containing |
| Disease | courses of intensive | higher doses of cytarabine |
| | induction treatment; | are generally considered as |
| | excluding patients with | the best option for patients |
| | death in aplasia or death | not responding to a first |
| | due to indeterminate cause | cycle of 7 + 3; the |
| | | likelihood of responding to |
| | | such regimens is lower |
| | | after failure of a first |
| Death in aplasia | | |
| Death from indeterminate | Deaths occurring before | |
| cause | completion of therapy, | |
| | or <7 days following its | |
| | completion; or deaths | |
| | occurring ≥7 days | |
| | following completion of | |
| | initial therapy with no | |
| | blasts in the blood, but no | |
| | bone marrow examination | |
| | available | |

Response criteria for clinical trials only

| | | |
|---|---|---|
| Stable disease | Absence of CR, CRi, PR, | Period of stable disease |
| | MLFS; and criteria for | should last at least 3 |
| | Progressive Disease | months. |
| | not met | |
| Progressive disease | Evidence for an increase in | Category mainly applies |
| (PD)^{a, b} | bone marrow blast | for older participants given |
| | percentage and/or increase | low-intensity or single- |
| | of absolute blast counts in | agent “targeted therapies” |
| | the blood: | in clinical trials |
| | >50% increase in marrow | In general, at least 2 cycles |
| | blasts over baseline (a | of a novel agent should be |
| | minimum 15% point | administered |
| | increase is required in | |
| | cases with <30% blasts at | |
| | baseline); or persistent | |
| | marrow blast percentage | |
| | of >70% over at least 3 | |
| | months.; without at least a | |
| | 100% improvement in | |
| | ANC to an absolute level | |
| | (>0.5 × 10⁹/L [500/μL], | |
| | and/or platelet count | |
| | to >50 × 10⁹/L [50,000/μL] | |
| | nontransfused) or | |
| | >50% increase in | Some protocols may |
| | peripheral blasts (WBC × | require blast increase in 2 |
| | % blasts) to >25 × 10⁹/L | consecutive marrow |
| | (>25,000/μL) (in the | assessments at least 4 |
| | absence of differentiation | weeks apart; the date of |
| | syndrome)^{b} or | progression should then be |
| | | defined as of the first |
| | | observation date |
| | New extramedullary | Some protocols may allow |
| | disease | transient addition of |
| | | hydroxyurea to lower blast |
| | | counts |
| | | Some protocols may allow |
| | | transient addition of |
| | | hydroxyurea to lower blast |
| | | counts |
| Relapse | Bone marrow blasts ≥5%; | |
| | or reappearance of blasts | |
| | in the blood; or | |
| | development of extra | |
| | medullary disease. | |

^{a}This new provisional category is arbitrarily defined; the category aims to harmonize the various definitions used in different clinical trials.
^{b}Certain targeted therapies, for example, those inhibiting mutant IDH proteins, may cause a differentiation syndrome, that is, a transient increase in the percentage of bone marrow blasts and an absolute increase in blood blasts; in the setting of therapy with such compounds, an increase in blasts may not necessarily indicate progression of disease.
Source: [Dohner, H., et al. 2017]

TABLE 11
Criteria for measurement of treatment response in adult MDS/MPN

CR (presence of all of the following improvements) ^{a}
Bone marrow: ≤5% myeloblasts (including monocytic blast equivalent in case of CMML)
with normal maturation of all cell lines and return to normal cellularity ^{a}
Osteomyelofibrosis absent or equal to “mild reticulin fibrosis” (≤Grade 1 fibrosis) ^{b}
Peripheral blood ^{c}
WBC ≤10 × 10⁹ cells/L
Hgb ≥11 g/dL
Platelets ≥100 × 10⁹/L; ≤450 × 10⁹/L
Neutrophils ≥1.0 × 10⁹/L
Blasts 0%
Neutrophil precursors reduced to ≤2%
Monocytes ≤1 × 10⁹/L
Extramedullary disease: Complete resolution of extramedullary disease present before
therapy (e.g., (e.g., cutaneous disease, disease-related serous effusions), including palpable
hepatosplenomegaly
Provisional category of CR with resolution of symptoms: ^{c} CR as described above, and
complete resolution of disease-related symptoms as noted by the MPN-SAF TSS
Persistent low-level dysplasia is permitted given subjectivity of assignment of dysplasia ^{a}
Complete cytogenetic remission
Resolution of previously present chromosomal abnormality (known to be associated with
myelodysplastic, syndrome myeloproliferative neoplasms, or MDS/MPN), as seen on
classic karyotyping with minimal of 20 metaphases or FISH ^{d}
Partial remission
Normalization of peripheral counts and hepatosplenomegaly with bone marrow blasts (and
blast equivalents) reduced by 50%, but remaining >5% of cellularity except in cases of
MDS/MPN with ≤5% bone marrow blasts at baseline
Marrow response
Optimal marrow response: Presence of all marrow criteria necessary for CR without
normalization of peripheral blood indices as presented above
Partial marrow response: Bone marrow blasts (and blast equivalents) reduced by 50%, but
remaining >5% of cellularity, or reduction in grading of reticulin fibrosis from baseline on
at least 2 bone marrow evaluations spaced at least 2 months apart
Clinical benefit
Requires 1 of the following in the absence of progression or CR/partial response and
independent of marrow response (cord blood response must be verified at ≥8 wk) to be
considered a clinical benefit
Erythroid response
Hgb increase by ≥2.0 g/dL
TI for ≥8 wk for patients requiring at least 4 packed red blood cell transfusions in the
previous 8 wk
Only red blood cell transfusions given based on physician's judgment for a
pretreatment Hgb of ≤8.5 g/dL will count in the red blood cell TI response evaluation ^{e}
Platelet response
Transfusion independence when previously requiring platelet transfusions of at least a
rate of 4 platelet transfusions in the previous 8 wk
Pretreatment ≤20 × 10⁹/L: increase from <20 × 10⁹/L to >20 × 10⁹/L and by at least
100%
Pretreatment >20 × 10⁹/L but ≤100 × 10⁹/L: absolute increase of ≥30 × 10⁹/L ^{e}
Neutrophil response
Pretreatment ≤0.5 × 10⁹/L at least 100% increase and an absolute increase ≥0.5 ×
10⁹/L
Pretreatment, >0.5 × 10⁹/L and ≤1.0 × 10⁹/L At least 50% increase and an absolute
increase
≥0.5 × 10⁹/L ^{e}
Spleen response
Either a minimum 50% reduction in palpable splenomegaly of a spleen that is at least
10 cm at baseline or a spleen that is palpable at more than 5 cm at baseline becomes
not palpable
Symptom response
Improvement in symptoms as noted by decrease of ≥50% as per the MPN-SAF TSS
scoring <20 were not considered eligible for measuring clinical benefit. ^{f}
Combination of 2 major criteria, 1 major and 2 minor criteria, or 3 minor criteria
from list
Major criteria
Increase in blast count ^{g}
<5% blasts: ≥50% increase and to >5% blasts
5-10% blasts: ≥50% increase and to >10% blasts
10-20% blasts: ≥50% increase and to >20% blasts
20-30% blasts: ≥50% increase and to >30% blasts ^{h}
Evidence of cytogenetic evolution ⁱ
Appearance of a previously present or new cytogenetic abnormality in complete
cytogenetic remission via fluorescence in situ hybridization (FISH) or classic
karyotyping
Increase in cytogenetic burden of disease by ≥50% in partial cytogenetic remission via
FISH or classic karyotyping
New extramedullary disease
Worsening splenomegaly
Progressive splenomegaly that is defined by International Working Group for
Myelofibrosis Research and Treatment (IWG-MRT): the appearance of a previously
absent splenomegaly that is palpable at >5 cm below the left costal margin or a
minimum 100% increase in palpable distance for baseline splenomegaly of 5-10 cm or
a minimum 50% increase in palpable distance for baseline splenomegaly of >10 cm
Extramedullary disease outside of the spleen
To include new/worsening hepatomegaly, granulocytic sarcoma, skin lesions, etc.
Minor criteria
Transfusion dependence ^{j}
Significant loss of maximal response on cytopenias ≥50% decrement from maximum
remission/response in granulocytes or platelets
Reduction in hemoglobin (Hgb) by ≥1.5 g/dL from best response or from baseline as noted
on complete blood count
Increasing symptoms as noted by increase in ≥50% as per the myeloproliferative neoplasm
symptom assessment form (MPN-SAF) total symptom score (TSS) ^{k}
Evidence of clonal evolution (molecular) ^{l}
^{a} Presence of dysplastic changes, which may be interpreted within the scope of normal range of dysplastic changes, may still exist in the presence of CR as allowed in MDS IWG. Marrow should exhibit age-adjusted normocellularity in CR.
^{b} If there is no significant fibrosis present on the initial bone marrow biopsy, a second biopsy is not required to prove resolution of fibrosis. Grading of fibrosis in measurement of treatment response should be according to the European Consensus System.
^{c} Given the current lack of a validated tool to assess complete resolution of symptoms in MDS/MPN, “CR with resolution of symptoms” (a complete resolution of disease-related symptoms as noted by the MPN-SAF TSS in presence of CR) will be a provisional category of disease response.
^{d} Loss of cytogenetic burden of disease by (via FISH or classic karyotyping) known to adversely affect prognosis is required to reach complete cytogenetic remission. Decrease in the cytogenetic burden of disease must be by ≥50% (via FISH or classic karyotyping) to be indicative of a partial cytogenetic response. Given variability of fluorescent probes used in FISH, cytogenetic normalization via FISH will depend on the performance characteristics of the specific probes used.
^{e} Resolution of abnormal peripheral blood counts must persist for at least 2 separate analyses over at least 8 wk. In the case of proliferative MDS/MPN, CR will include resolution of thrombocytosis to a normal platelet count (150-450 × 10⁹/L) and resolution of leukocytosis to WBC ≤10 × 10⁹ cells/L but ≥1.5 × 10⁹/L. Hgb should be maintained >11 g/dL and platelets ≥100 × 10⁹/L without the support of transfusions. Clinical benefit may occur when these changes occur in absence of other changes required for CR or marrow response. Platelet and packed red blood cell TI would be considered for clinical benefit, and duration of TI should be monitored. Reduction in myeloid precursors (promyelocytes, myelocytes, metamyelocytes, nucleated red blood cells) to less than appreciable levels (≤2-3%) and/or 1 × 10⁹/L monocytosis in the absence of infection, cytokine treatment, or other reactive causes.
^{f} MPN-SAF TSS validation among patients with MDS/MPN is currently under way (R.A. Mesa, personal communication, 2014).
^{g} Blasts as measured from the bone marrow.
^{h} Patients with development of acute myeloid leukemia from MDS/MPN; 20-30% blasts may be allowed on some clinical trials for patients with MDS/MPN.
ⁱ Increase in cytogenetic burden of disease by ≥50% (via FISH or classic karyotyping). Given variability of fluorescent probes used in FISH, cytogenetic normalization via FISH will depend on specific probes used.
^{j} Transfusion dependency is defined by a history of at least 2 U of red blood cell transfusions in the past month for a hemoglobin level <8.5 g/dL that was not associated with clinically overt bleeding. Cytopenias resulting from therapy should not be considered in assessment of progression.
^{k} MPN-SAF TSS validation among patients with MDS/MPN is currently under way (R.A. Mesa, personal communication, 2014).
^{l} The identification of new abnormalities using single nucleotide polymorphism arrays or sequencing or a clearly significant increase in mutational burden of a previously detected abnormality. Precise criteria for defining new abnormalities and what exactly constitutes a significant increase in mutational burden are open to interpretation; we suggest that this criterion should be used conservatively based on current evidence.
Source: Adapted from [Savona, M. R., et al. 2015]

Overall survival is defined for all participants of atrial measured from the date of entry into a clinical trial or from the date of diagnosis (e.g., for correlative science studies) to the date of death from any cause, patients not known to have died at last follow-up are censored on the date they were last known to be alive [Dohner, H., et al. 2017].

Intraparticipant dose escalation is allowed for participants who are enrolled into the first 2 dose levels of Part 1 once they have completed DLT evaluation and once a higher dose level has been cleared for DLT if the participants have not progressed.

Anti-ILT3 antibody will be administered via IV infusion in a 3-week cycle. Participants will be treated until progressive disease, unacceptable toxicity, intercurrent illness that prevents further administration of treatment, investigator's decision to withdraw treatment, participant withdrawal of consent, pregnancy of the participant, noncompliance with study intervention or procedure requirements, participant completes treatment, or administrative reasons requiring cessation of treatment. Participants may receive study treatment for up to 35 cycles (24 months). In addition, if a participant has not achieved a partial or complete remission after 6 months of study treatment, the investigator should discuss the lack of response to the study treatment and other treatment options with the participant. If other alternative treatments with potential clinical benefits are available for the participant at that time, study treatment should be discontinued.

Participants who discontinue treatment for reasons other than confirmed progressive disease will be followed for disease status until disease progression, initiating a new anticancer therapy, withdrawing consent for study participation, or becoming lost to follow-up.

After confirmed progressive disease, each participant will be contacted by telephone every 12 weeks (84±14 days) for survival follow-up until withdrawal of consent to participate in the study, becoming lost to follow-up, death, or end of the study, whichever occurs first.

### Efficacy Endpoints

Since this is a Phase 1b study, clinical responses are included for efficacy evaluations as secondary endpoints, including rate of CR, rate of composite CR (CR+CRi) and objective response rate (CR+CRi and PR). The response criteria for AML as defined in the 2017 ELN international expert panel recommendations (see Table 10) are well adapted in the clinical field worldwide, which also include response parameters suitable for clinical studies such as definition of stable disease, progressive disease, and relapse etc. The assessments of these parameters are developed in accordance with the 2016 WHO classification of myeloid neoplasms and acute leukemia [Arber, D. A., et al. 2016]. The response criteria for CMML will be evaluated per 2015 International Consortium Proposal of Uniform Response Criteria for MDS/MPN in Adults as outlined in Table 11.

### Safety Endpoints

The primary objective of this study is to characterize the safety and tolerability of anti-ILT3 antibody as monotherapy. The primary safety analysis will be based on participants who experience toxicities as defined by NCI CTCAE Version 5.0 criteria. Safety will be assessed by quantifying the toxicities and grades of toxicities experienced by participants who have received anti-ILT3 antibody as monotherapy.

For AEs, attribution to drug, time-of-onset, duration of the event, its resolution, and any concomitant medications administered will be recorded. AEs that will be analyzed include, but are not limited to, all AEs, SAEs, fatal AEs, and laboratory changes.

### Pharmacokinetic Endpoints

A secondary objective of this study is to characterize the PK profile of anti-ILT3 antibody after administration as a single agent. The serum concentration of this agent will serve as the primary readout for the PK, and these data will be used to derive PK parameters of the agent. Furthermore, the results of these analyses will be used in conjunction with the pharmacodynamics, and safety and exploratory endpoint data to help assess future dosing strategies for anti-ILT3 antibody.

### Antidrug Antibodies

Formation of ADA can potentially confound drug exposures at therapeutic doses and prime for subsequent infusion-related toxicity. Antidrug antibody response at the beginning of each cycle will be determined to understand drug metabolism, exposure, and safety. The incidence of ADA and neutralizing antibodies (if applicable) will be evaluated and summarized over time by dose. Correlations between the presence/absence of positivity for ADAs and PK and pharmacodynamic markers, activity, and safety of anti-ILT3 antibody will be explored.

### Pharmacodynamic Endpoints

An exploratory objective of this study is to evaluate target engagement which will be used in conjunction with safety, PK, and additional pharmacodynamics biomarker data to guide dose escalation decisions and determine a RP2D. Target engagement will be assessed using a receptor occupancy assay that directly measures anti-ILT3 antibody binding to ILT3 on circulating CD14⁺ myeloid cells in peripheral blood and compares the receptor occupancy pre-administration and post-administration. In addition, receptor occupancy may be measured in bone marrow blasts if samples are adequate.

As preclinical evidence suggests a dose-dependent relationship between sILT3 concentrations and target binding, sILT3 will be measured using an enzyme-linked immunoassay, and the correlation of sILT3 levels with anti-ILT3 antibody treatment will be evaluated.

### Rationale for Starting and Maximum Dose of Anti-ILT3 Antibody

Anti-ILT3 antibody Q3W has been evaluated in advanced solid tumors as monotherapy at dose levels ranging from 0.2 mg to 2250 mg; and in combination with pembrolizumab 200 mg Q3W in dose levels ranging from 7.5 mg to 2250 mg during a previous clinical trial. Anti-ILT3 antibody was well tolerated in all the dose levels in monotherapy and had an acceptable safety profile in combination with pembrolizumab.

Preliminary PK data for the solid tumor clinical trial showed target-mediated drug disposition at lower anti-ILT3 antibody doses while linear PK was observed at tested doses≥75 mg. Near complete receptor occupancy was also observed in blood samples from participants treated with anti-ILT3 antibody at dose levels≥75 mg. Even with stringent assumptions, 750 mg anti-ILT3 antibody Q3W is likely to maintain complete receptor occupancy in the tumor.

While ADA was observed in 16 of 62 participants with evaluable data treated with anti-ILT3 antibody doses between 0.2 mg and 750 mg, there was no clear impact of ADA on PK or receptor occupancy. A dose-dependent increase in total soluble ILT3 (sILT3) concentration was seen in blood samples; however, based on internal investigations, there was no confirmed immunosuppressive activity for soluble ILT3.

ILT3 target expression levels in AML/CMML patient blood, relative to patients in other solid tumors is unknown. In AML/CMML patients, the safety profile resulting from ILT3 target binding is also unknown. Therefore, dose escalation will start at 7.5 mg to rule out any unforeseen adverse events. In patients with solid tumors, this dose yields minimal target engagement in blood at trough concentration (˜20%). This study will enroll 3 to 6 participants initially for each cohort at 75 mg, 225 mg, and 750 mg dose levels and will increase up to 10 participants as needed per mTPI design. Trough target engagement increases substantially between 7.5 and 75 mg in patients with solid tumors, and thus safety evaluations in more participants is warranted beyond 25 mg.

Based on the collective evaluation of data from safety, PK, and receptor occupancy, the 750 mg dose of anti-ILT3 antibody was selected as the RP2D in combination with pembrolizumab for further evaluation in advanced solid tumors. Complete target engagement is expected to be achieved by this dose; however, based on actual data from the dose escalation, a higher dose level may be evaluated, if warranted.

### Rationale for Dose Interval and Escalation Increments

Once complete target engagement is achieved, anti-ILT3 antibody exhibits a PK profile that is consistent with that of other monoclonal antibodies. Preliminary data from a study of anti-ILT3 antibody in solid tumors suggests that anti-ILT3 antibody has a half-life of approximately 17 days. A 3-week dose interval is expected to be adequate to maintain complete target engagement at trough in AML/CMML patients.

Approximately 3-fold dose escalation increments will be used. While the extent of population variability in exposure in AML/CMML patients is not known, a 3-fold difference between doses is expected to produce nonoverlapping exposures across doses.

### Accelerated Titration Design

The initial dose escalation will follow an ATD to minimize the number of participants treated at potentially subtherapeutic doses of anti-ILT3 antibody. Single participants will be enrolled sequentially into the escalating dose levels 7.5 mg and 25 mg, respectively. The transition from ATD to mTPI is planned at the next dose level of 75 mg.

Intraparticipant dose escalation will be allowed for participants in the ATD. Participants may undergo dose escalation up to the 75 mg dose level. Intermediate dose levels may be evaluated, if warranted. The dose to be tested in each group of participants will be communicated to the investigators or designees after the dose-escalation decision meeting for the previous dose. Enrollment of up to 3 participants per dose level at ATD is permitted on approval by the Sponsor's medical monitor or designee provided that the first 2 participants will receive anti-ILT3 antibody treatment at least 3 days apart. All participants enrolled at each dose level must complete the DLT period before the next dose level is initiated.

The ATD will end when at least 1 of the following occurs:
The highest dose level (up to 75 mg) has completed the DLT evaluation period and anti-ILT3 antibody has been determined to be safe and well tolerated in this cohort.
Occurrence of a Grade 2 or higher treatment-related toxicity according to NCI CTCAE 5.0 during Cycle 1 (ATD ends at that current dose level).

Any time a DLT occurs in the ATD phase, the dose level in which the DLT occurred will be expanded at this dose per mTPI guidelines below. If no DLT occurs in the ATD phase, then the ATD phase will proceed to the mTPI phase once 1 of the above triggers is met.

### Dose Finding Using a Modified Toxicity Probability Interval Design

Further dose finding will follow the mTPI design [Ji Y et al. 2007] with a target DLT rate of 25%. Dose escalation and de-escalation decisions are based on the mTPI design and depend on the number of participants enrolled and number of DLTs observed at the current dose level.

A minimum of 3 participants are required at each dose; however, depending on the accrual rate, 3 to 6 participants may be enrolled to an open dose level providing that the first participants receive the first dose at least 3 days apart. In Table 12, the columns indicate the numbers of participants treated at the current dose level, and the rows indicate the numbers of participants experiencing DLT. The entries of the table are the dose-finding decisions: E, S, D, and DU represent escalating the dose, staying at the same dose, de-escalating the dose, and excluding the dose from the study due to unacceptable toxicity, respectively. For example, if 0 of 3 participants at a given dose level develop a DLT, then the dose can escalate to the next level. If 2 participants of 3 develop a DLT, the dose will be de-escalated to the next lower dose level. If 3 of 3 participants develop a DLT, this indicates an unacceptable toxicity at this dose. The dose should be de-escalated, and the current dose will not be explored further. If 1 of 3 participants at a given dose level develop a DLT, then additional participants should be enrolled at that dose level following the rules below.

When adding participants to a dose level in response to a “stay” decision, the number of additional participants to be enrolled is capped to minimize the exposure to a dose that may be unacceptably toxic (denoted as DU in Table 12). Second, to determine how many more participants can be enrolled at the dose level, one can count steps in a diagonal direction (down and to the right) from the current cell to the first cell marked DU. For example, if 1 of 3 participants experienced a DLT at a given dose level, no more than an additional 3 participants should be enrolled at this dose level until additional DLT data are available. This dose level would be considered unacceptably toxic if all 3 of the additional participants experience a DLT (i.e., 4/6 participants with DLT in Table 12). The same principles will be applied whether 3, 4, 5, or 6 participants are initially enrolled at that dose level.

A D or DU decision at the lowest dose level will stop the study. An E decision at the highest dose level will result in staying at that level. During dose finding, it may be acceptable to de-escalate to an intermediate dose that was not predefined and not previously studied if evaluation of toxicity at such a dose is desired. If this approach is taken, 3 to 6 new participants may be enrolled at the new intermediate dose, and the aforementioned rules should be used to determine further enrollment at this dose level.

After 10 participants have been enrolled at any of the tested doses (including intermediate doses), dose finding will stop if the mTPI table indicates “S” for staying at current dose. Otherwise, up to 10 new participants may be enrolled at a lower dose if “D” or “DU” is indicated, or at a higher dose if “E” is indicated.

The pool-adjacent-violators algorithm [Ji, Y. et al. 2013] will be used to estimate the DLT rates across doses. The dose with an estimated DLT rate closest to 25% will be treated as a preliminary MTD. However, the totality of the data will be considered before deciding on the dose to carry forward to Part 2, and the escalation schedule may be adjusted based on pharmacodynamic, PK, and safety data emerging throughout the study.

Note that although 25% was the target toxicity rate used to generate the guidelines in Table 12, the observed rates of participants with DLTs at the MTD may be slightly above or below 25%.

TABLE 12
Dose-finding Rules per mTPI Design

| Number of | | | | | | | | |
|---|---|---|---|---|---|---|---|---|
| participants | | | | | | | | |
| with at | | | | | | | | |
| least 1 DLT | 3 | 4 | 5 | 6 | 7 | 8 | 9 | 10 |
| 0 | E | E | E | E | E | E | E | E |
| 1 | S | S | S | S | E | E | E | E |
| 2 | D | D | S | S | S | S | S | S |
| 3 | DU | DU | DU | D | S | S | S | S |
| 4 | | DU | DU | DU | DU | DU | D | S |
| 5 | | | DU | DU | DU | DU | DU | DU |
| 6 | | | | DU | DU | DU | DU | DU |
| 7 | | | | | DU | DU | DU | DU |
| 8 | | | | | | DU | DU | DU |
| 9 | | | | | | | DU | DU |
| 10 | | | | | | | | DU |

D = de-escalate to the next lower dose;
DLT = dose-limiting toxicity;
DU = the current dose is unacceptably toxic;
E = escalate to the next higher dose;
mTPI = modified toxicity probability interval;
S = stay at the current dose;
Target toxicity rate = 25%
Flat noninformative prior Beta (1, 1) is used as a prior and ε1 = ε2 = 0.03 [Ji Y et al. 2007] [Ji, Y. et al. 2013] [Ji, Y., et al. 2010]

### Clinical Criteria for Early Study Termination

Recruitment in the study or at particular study site may be stopped due to insufficient compliance with the protocol, GCP, and/or other applicable regulatory requirements, procedure-related problems or the number of discontinuations for administrative reasons is too high.

Early study termination will be the result of the criteria specified below:
1. Incidence or severity of adverse drug reactions in this or other studies suggest a potential health hazard to participants
2. Plans to modify or discontinue the development of the study medication

Ample notification will be provided in the event of Sponsor decision to no longer supply anti-ILT3 antibody.

### Study Population

Male/female participants at least 18 years of age with relapsed or refractory AML or CMML will be enrolled in this study.

This study includes participants of varying age (as applicable), race, ethnicity, and sex (as applicable). The collection and use of these demographic data will follow all local laws and participant confidentiality guidelines while supporting the study of the disease, its related factors, and the investigational medicinal product under investigation.

Prospective approval of protocol deviations to recruitment and enrollment criteria, also known as protocol waivers or exemptions, is not permitted.

### Inclusion Criteria

A participant will be eligible for inclusion in the study if the participant:

1. Has a confirmed diagnosis of AML with myelomonocytic or monoblastic/monocytic differentiation per WHO 2016 criteria and with confirmed refractory or relapsed disease (i.e., ≥5% blast in bone marrow or in peripheral blood) after treatment with available therapies known to benefit participant's AML subtypes;
OR
has known diagnosis of CMML per WHO criteria [Orazi, A., et al. 2017] with confirmed refractory or relapsed disease after treatment with available therapies known to be active for CMML.

2. Has a WBC count≤20×10⁹/L within 24 hours prior to the first dose of study treatment. Note: Hydroxyurea should be used to keep the WBC count maintained≤20×10⁹/L until the first dose of study treatment, to the extent that this is possible.

3. Has an ECOG performance status of 0 to 2 as assessed within 72 hours prior to the first dose of study treatment.

4. Has adequate organ function as defined in Table 13 below and as assessed within 72 hours prior to the first dose of study treatment.

TABLE 13
Adequate Organ Function Laboratory Values

| | |
|---|---|
| System | Laboratory Value |

Renal

| | |
|---|---|
| Creatinine OR | ≤1.5 × ULN or |
| Measured or calculated^{a} CrCl | ≥40 mL/min for participants with |
| (GFR can also be used in place of CrCl) | creatininelevels >1.5 × ULN |

Hepatic

| | |
|---|---|
| Total bilirubin | ≤1.5 × ULN OR direct bilirubin ≤ULN or |
| | ≤3 × ULN if deemed to be elevated due |
| | toGilbert's disease or leukemia |
| AST (SGOT) and ALT (SGPT) | ≤3 × ULN |

^{a}CrCl should be calculated per institutional standard.
Note:
This table includes eligibility-defining laboratory value requirements for treatment; laboratory valuerequirements should be adapted according to local regulations and guidelines for the administration of specific chemotherapies.

5. Is male or female, at least 18 years at the time of providing documented informed consent.

6. Is not pregnant or breastfeeding, and at least one of the following conditions applies:
Is not a WOCBP OR
Is a WOCBP and
   uses a contraceptive method that is highly effective (with a failure rate of <1% per year), or be abstinent from heterosexual intercourse as their preferred and usual lifestyle (abstinent on a long-term and persistent basis), during the intervention period and for at least 90 days after the last dose of study intervention. The investigator should evaluate the potential for contraceptive method failure (i.e., noncompliance, recently initiated) in relationship to the first dose of study intervention. Contraceptive use by women should be consistent with local regulations regarding the methods of contraception for those participating in clinical studies. Has a negative highly sensitive pregnancy test (urine, as required by local regulations) within 72 hours before the first dose of study intervention. If a urine test cannot be confirmed as negative (e.g., an ambiguous result), a serum pregnancy test is required. In such cases, the participant must be excluded from participation if the serum pregnancy result is positive.
   Abstains from breastfeeding during the study intervention period and for at least 90 days after study intervention.
   medical history, menstrual history, and recent sexual activity has been reviewed by the investigator to decrease the risk for inclusion of a woman with an early undetected pregnancy.

7. The participant (or legally acceptable representative) has provided documented informed consent for the study. The participant may also provide consent for future biomedical research. However, the participant may participate in the main study without participating in future biomedical research.

8. Has a bone marrow aspirate and biopsy sample performed within 14 days of treatment start date.

### Exclusion Criteria

The participant must be excluded from the study if the participant:

1. Has active CNS leukemia. Note: Participants with clinical signs of CNS involvement or with suspected CNS involvement must have CSF testing to confirm leukemic involvement.

2. Has isolated extramedullary disease, i.e., no leukemic involvement in bone marrow or peripheral blood.

3. Has diagnosis of acute promyelocytic leukemia or participants with known Ph+AML.

4. Has received previous allogeneic stem cell transplant or organ transplant within 60 days of the start of study treatment. Note: Participants with relapsed AML or CMML after allogeneic SCT, including those who have received donor lymphocyte infusions, are eligible if they have no active graft versus host disease (GVHD) and are off immunosuppression therapy or are taking a maintenance dose of <10 mg daily prednisone or equivalent. Note: Receipt of previous autologous transplant for AML or non-AML condition is allowed.

5. Has a history of a second malignancy, unless potentially curative treatment has been completed with no evidence of malignancy for 1 year. Note: The time requirement does not apply to participants who underwent successful definitive resection of basal cell carcinoma of the skin, squamous cell carcinoma of the skin, superficial bladder cancer, or carcinoma in situ (e.g., breast cancer in situ, cervical cancer in situ).

6. Has a history of any of the following cardiovascular conditions within 6 months of screening: myocardial infarction, unstable angina, cerebrovascular accident, transient ischemic attack, coronary artery bypass graft, or pulmonary embolism; has New York Heart Association (NYHA) Class III or IV congestive heart failure.

7. Has had a severe hypersensitivity reaction to treatment a mAb and or any components of the study intervention, anti-ILT3 antibody.

8. Has an active uncontrolled infection requiring directed therapy.

9. Has immediately life-threatening, severe complications of leukemia such as uncontrolled bleeding, pneumonia with hypoxia or shock, or disseminated intravascular coagulation.

10. Has known HIV and/or hepatitis B or C infections, or is known to be positive for HBsAg/HBV DNA or hepatitis C antibody or RNA. Active hepatitis C is defined by a known positive Hep C Ab result and known quantitative HCV RNA results greater than the lower limits of detection of the assay.

11. Has known psychiatric or substance abuse disorders (verbally reported) that would interfere with the participant's ability to cooperate with the requirements of the study.

12. Is pregnant or breast feeding or expecting to conceive or father children within the projected duration of the study, starting with the Screening Visit through 120 days after the last dose of study intervention.

13. Has received systemic anticancer therapy, radiotherapy, or surgery within 2 weeks before the start of study treatment. Note: Participants must have recovered from all AEs due to previous therapies to ≤Grade 1 or baseline.

14. Has received hematopoietic cytokines (G-CSF, GM-CSF, or erythropoietin) within 2 weeks prior to start of study treatment.

15. Has received a live or live attenuated vaccine within 30 days before the first dose of study medication. Note: Killed vaccines are allowed.

16. Has received prior treatment(s) with another agent targeting ILT3.

17. Is currently participating and receiving study intervention in a study of an investigational agent or has participated and received study intervention in a study of an investigational agent or has used an investigational device within 14 days of administration of anti-ILT3 antibody. Note: Participants who have entered the follow-up phase of an investigational study may participate as long as it has been 2 weeks since the last dose of the previous investigational agent.

18. Has a diagnosis of immunodeficiency or is receiving chronic systemic steroid therapy (in dosing exceeding 10 mg daily of prednisone equivalent) or any other form of immunosuppressive therapy within 7 days prior the first dose of study medication. Note: Participants who require intermittent use of non-systemic steroids such as ocular, inhaled, intranasal, topical steroids, or local steroid injections are not excluded from the study.

### Screen Failures

Screen failures are defined as participants who consent to participate in the clinical study, but are not subsequently entered in the study. A minimal set of screen-failure information is required to ensure transparent reporting of screen-failure participants to meet the CONSORT publishing requirements and to respond to queries from regulatory authorities. Minimal information includes demography, screen-failure details, eligibility criteria, and any AEs or SAEs meeting reporting requirements as outlined in the data entry guidelines.

### Participant Replacement Strategy

To adequately evaluate the safety of the doses administered in this study, all participants enrolled must meet the criteria for evaluability for Cycle 1. Participants are considered non-evaluable for DLT evaluation if:
They are allocated, but not treated.
They discontinue from the study before completing all the safety evaluations for reasons other than treatment-related AEs.
They receive <75% of the total anti-ILT3 antibody infusion in Cycle 1 (e.g., if the infusion had to be discontinued due to an infusion reaction) and did not experience a DLT.

Participants who are non-evaluable for DLT evaluation will be replaced unless accrual at the dose level has stopped. Non-evaluable participants will not be counted toward the total number of participants at the dose level for DLT evaluation.

If a participant experiences a DLT in Cycle 1, study intervention may be discontinued; however, if the participant is deriving clinical benefit from the study intervention, the participant may be allowed to continue after discussion with and approval by the Sponsor.

### Intervention Assignment

In Part 1 of the study, treatment will be allocated by nonrandom assignment using an IVRS/IWRS based on the dose level evaluated at the time. C1D1 treatment for the first and second enrolled participants should be at least 3 days apart. A new dose level group will not start until the previous dose level group has been evaluated for DLT and is indicated for dose escalation. Part 2 enrollment will be initiated after the RP2D dose is determined and treatment will be allocated by nonrandom assignment using an IVRS/IWRS.

### Acceptable Concomitant Medications

All treatments that the investigator considers necessary for a participant's welfare may be administered at the discretion of the investigator in keeping with the community standards of medical care except for those that are prohibited as described in Section 6.5.2. All concomitant medication will be recorded on the CRF including all prescription, OTC, herbal supplements, and IV medications and fluids. If changes occur during the study period, documentation of drug dosage, frequency, route, and date may also be included on the CRF.

All concomitant medications received within 30 days prior to the first dose of study intervention and up to 30 days after the last dose of study intervention should be recorded. If participants experience an SAE or ECI, all concomitant medications administered after 30 days after the last dose of study intervention are to be recorded.

### Prohibited Concomitant Medications

Participants are prohibited from receiving the following therapies during the screening and treatment phases of this study:
Antineoplastic systemic chemotherapy or biological therapy
Immunotherapy not specified in this protocol
Chemotherapy not specified in this protocol
Investigational agents
Radiation therapy

Note: Radiation therapy to a symptomatic solitary lesion or to the brain may be allowed at the investigator's discretion with Sponsor consultation after the DLT observation period for the participant to be considered evaluable for DLT.
Live or attenuated vaccines within 30 days before the first dose of study intervention and while participating in the study. Examples of live vaccines include, but are not limited to the following: measles, mumps, rubella, varicella/zoster, yellow fever, rabies, BCG, and typhoid vaccine. Seasonal influenza vaccines for injection are generally killed virus vaccines and are allowed; however, intranasal influenza vaccines (e.g., FluMist®) are live attenuated vaccines and are not allowed.

Note: Any licensed COVID-19 vaccine (including for Emergency Use) in a particular country is allowed in the study as long as they are mRNA vaccines, replication incompetent adenoviral vaccines, or inactivated vaccines. These vaccines will be treated just as any other concomitant therapy.
Systemic glucocorticoids for any purpose other than to modulate symptoms from an AE of suspected immunologic etiology. The use of physiologic doses of corticosteroids may be approved after consultation with the Sponsor.

Participants who, in the assessment by the investigator, require the use of any of the aforementioned treatments for clinical management should be discontinued from study intervention. Participants may receive other medications that the investigator deems to be medically necessary.

### General Supportive Care

Supportive care for managing AML should be given as needed per institution standard such as transfusion of leukocyte-depleted blood products (e.g., RBC, platelets), prophylaxis and treatment for infections. Hydroxyurea can be given in an attempt to maintain WBC to ≤20×10⁹/L. Growth factors (GM-CSF, G-CSF) may be considered as a part of supportive care for post-remission therapy; however, it may confound bone marrow evaluation and therefore should be off for a minimum of 7 days before obtaining bone marrow for evaluation.

### Tumor Lysis Prophylaxis

Participants with risk for developing TLS should receive prophylaxis treatment, such as with allopurinol, extra hydration, and diuretics, etc. per institution standard as clinically indicated. Hydroxyurea can be given in an attempt to maintain WBC to <20×10⁹/L during treatment (see above). Classification of Tumor Lysis Syndrome is summarized in Table 14 below.

TABLE 14
Classification of Tumor Lysis Syndrome

| | Laboratory TLS | Clinical TLS |
|---|---|---|
| Metabolic Abnormality | Classification Criteria^{a} | Classification Criteria^{b} |
| Hyperuricemia | Uric acid ≥8 mg/dL | N/A |
| Hyperkalemia | Potassium ≥6 mmol/liter | Cardiac dysrhythmia or |
| | | sudden death probably or |
| | | definitely caused by |
| | | hyperkalemia |
| Hyperphosphatemia | Phosphorous ≥4.5 mg/dl | N/A |
| Hypocalcemia | Corrected calcium ≤7.0 | Cardiac dysrhythmia, |
| | mg/dL orionized calcium | sudden death, seizure, |
| | <1.12 mg/dL^{c} | neuromuscular irritability |
| | | (tetany, paresthesia, |
| | | muscle twitching, |
| | | carpopedal spasm, |
| | | Trousseau's sign, |
| | | Chvostek's sign, |
| | | laryngospasm, or |
| | | bronchospasm), |
| | | hypotension, or heart |
| | | failure probably or |
| | | definitely caused by |
| | | hypocalcemia |
| Acute Kidney Injury^{d} | N/A | Increase in the serum |
| | | creatininelevel of 0.3 |
| | | mg/dL or the presence of |
| | | oliguria (average urine |
| | | output of <0.5 mL/kg/h |
| | | over a 6-hour period) |

N/A = not available;
TLS = tumor lysis syndrome
^{a}Laboratory TLS requires 2 or more metabolic abnormalities must be present during the same 24-hour period within 3 days before the start of therapy or up to 7 days afterward.
^{b}Clinical TLS requires the presence of Laboratory TLS plus one or more findings from the Clinical TLS column.
^{c}Corrected calcium = measured calcium level in mg/dL + 0.8 × (4 − albumin in g/dL).
^{d}Acute kidney injury, unless attributable to another cause, represents clinical TLS even if criteria for laboratory TLS are not satisfied.
Source: [Howard, S. C., et al. 2011]

### Dose-Limiting Toxicity

All toxicities will be graded using NCI CTCAE 5.0 based on the investigator assessment.

The DLT window of observation will be 21 days since the first dose of study intervention (i.e., during Cycle 1).

The occurrence of any of the following toxicities during Cycle 1 will be considered a DLT, if assessed by the investigator to be possibly, probably, or definitely related to study intervention.

1. Any Grade 4 nonhematologic toxicity (not laboratory)

2. Any Grade 3 nonhematologic toxicity

### Exceptions to the DLT Definition:

Grade 3 fatigue lasting ≤3 days
Grade 3 diarrhea, nausea, or vomiting without requiring tube feeding, total parenteral nutrition, or prolonged hospitalization
Grade 3 hypersensitivity reaction that is successfully managed and resolved within 72 hours

3. Any Grade 3 or Grade 4 nonhematologic laboratory value if:
Clinically significant medical intervention is required to treat the participant, or
The abnormality leads to hospitalization, or
The abnormality persists for >1 week, or
Electrolyte imbalances lasting more than 48 hours despite optimal therapy, or
The abnormality results in a DILI

Exceptions to the DLT definition: Grade 3 or Grade 4 isolated abnormalities without clinical consequences that is resolved with or without intervention to less than Grade 2 in <72 hours.

4. Grade 4 neutropenia and/or thrombocytopenia, in the absence of active leukemia, lasting for more than 14 days.

5. Prolonged delay (>2 weeks) in initiating Cycle 2 due to intervention-related toxicity.

6. Any intervention-related toxicity that causes the participant to discontinue intervention during Cycle 1.

7. Missing >25% of anti-ILT3 antibody dose as a result of drug-related AEs during the first cycle.

8. Grade 5 toxicity.

### Dose Expansion

In Part 2 of the study, approximately 10 additional participants with AML will be enrolled with preliminary RP2D identified from Part 1.

### Timing of Dose Administration

anti-ILT3 antibody will be administered Q3W as an IV infusion. The reason for any variability in administration of anti-ILT3 antibody outside the protocol-specified window should be documented in the participant's medical record and recorded on the eCRFs.

Every effort should be made to begin the first dose of study intervention on the day of allocation or within 3 days of allocation. Subsequent doses will be administered on Day 1 of each cycle with a window of ±3 days.

### Dose Modification for Anti-ILT3 Antibody

The NCI CTCAE 5.0 must be used to grade the severity of AEs. The investigator may attribute each toxicity event to anti-ILT3 antibody and modify the dose according to Table 15. If a participant experiences several toxicities and there are conflicting recommendations, follow the most conservative recommendations. Exceptional circumstances to following the dose modification tables below may be considered after consultation with the Sponsor.

TABLE 15
anti-ILT3 antibody Dose Modification and Treatment Discontinuation
Guidelines for Drug-related Adverse Events

| | | | |
|---|---|---|---|
| | Hold/ | Criteria for | |
| | Discontinue | Restarting | Toxicity |
| Toxicity | Treatment | Treatment | Management |

Hematological Toxicities:

| | | | |
|---|---|---|---|
| Any Grade 4 | Discontinue | N/A | Symptomatic treatment |
| neutropenia or | | | and supportive care at |
| thrombocytopenia, | | | investigator's discretion |
| in the absence of | | | |
| active | | | |
| leukemia, lasting | | | |
| formore than 14 | | | |
| days. | | | |

Nonhematological Toxicities:

| | | | |
|---|---|---|---|
| Any Grade 1 | No | N/A | N/A |
| nonhematological | | | |
| toxicity | | | |
| Any Grade 2 | Dose | If treatment | Symptomatic |
| intolerant | interrupt | held, may be | treatment at |
| nonhematological | | restarted | investigator's |
| toxicity except | | when AE | discretion |
| Grade 2 fatigue | | resolves | |
| | | back to | |
| | | baseline or | |
| | | to Grade 1. | |
| Any Grade 3 | Dose | Treatment | Symptomatic |
| nonhematological | interrupt | may be | treatment at |
| toxicity, clinically | | restarted | investigator's |
| significant Grade 3 | | when AE | discretion |
| or 4 laboratory. | | resolves | |
| | | back to | |
| | | baseline or | |
| | | to Grade 1 | |
| | | and after | |
| | | Sponsor | |
| | | consultation | |
| Any Grade 4, or | Discontinue | N/A | Symptomatic |
| recurrent Grade 3 | | | treatment at |
| nonhematological | | | investigator's |
| toxicity | | | discretion |

AE = adverse event.; N/A = not applicable.

### Timing of Dose Administration

Dosing and schedules are summarized in Table 16 below.

TABLE 16
Summary of Study Interventions

| | | | | | Route | |
|---|---|---|---|---|---|---|
| | Inter- | Dose | Unit | | of | |
| Arm | vention | Formu- | Dose | Dosage | Admin- | |
| Name | Name | lation | Strength | Level(s) | istration | Regimen |
| Part 1 | anti-ILT3 | Solution | 100 mg/ | 7.5 mg, | IV | Q3W up |
| | antibody | for | vial | 25 mg, | Infusion | to 35 |
| | | Infusion | | 75 mg, | | cycles |
| | | | | 225 mg, | | |
| | | | | 750 mg | | |
| | | | | or higher | | |
| | | | | if needed | | |
| Part 2 | anti-ILT3 | Solution | 100 mg/ | RP2D | IV | Q3W up |
| | antibody | for | vial | | Infusion | to 35 |
| | | Infusion | | | | cycles |

After the first cycle, study intervention may be administered up to 3 days before or after the scheduled dosing date for each infusion due to administrative reasons.

On Day 1 of each cycle, anti-ILT3 antibody will be administered Q3W at the assigned dose level. Sites should make every effort to target infusion timing to be as close to 30 minutes as possible. Given the variability of infusion pumps form site to site, a window of minus (−) 5 minutes and plus (+) 10 minutes is allowed (i.e., infusion time is 30 minutes, −5 min/+10 min).

### AML or CMML Disease Assessments at Screening/Baseline

Disease status of participant's AML/CMML will be assessed by the investigator based on local laboratory reports. At screening/baseline, bone marrow aspirate and biopsy, peripheral blood samples will be collected for CBC and differentials, histopathology evaluation, and immunophenotyping (primarily focusing on acute myeloid and monocytic leukemic or CMML panels per institutional standard).

Participants with AML must have ≥5% blasts in bone marrow or peripheral blood at baseline to be eligible for the study. Blasts count will include myeloblasts, monoblasts, promonocytes, and/or megakaryoblasts per WHO criteria for AML [Dohner, H., et al. 2017].

Extramedullary disease should be evaluated as clinically indicated per institutional guideline. Participants with CNS leukemia or isolated extramedullary lesion (i.e., without bone marrow or peripheral disease as required per protocol) should be excluded. For eligible participants, locations of extramedullary lesions should be recorded in the CRF.

### AML or CMML Disease Assessments During Study Treatments

Disease status during the study treatment period will be evaluated by the investigator based on local laboratory reports of bone marrow and peripheral blood assessments.

Extramedullary disease will be evaluated or followed as clinically indicated. ELN 2017 Response evaluation criteria in AML and CMML as provided in Table 10 and Table 11, respectively, will be followed for evaluating disease status at each protocol-specified timepoint or as clinically indicated. Details in disease assessment will be recorded in the CRF.

### Eastern Cooperative Oncology Group Performance Scale

The investigator or qualified designee will assess ECOG status at screening, before the administration of each dose of study intervention on the day of study treatment, and during the follow-up period.

### Events of Clinical Interest (ECI)

Selected serious and nonserious AEs are also known as ECIs and must be reported to the Sponsor.

Events of clinical interest for this study include:
a. An overdose of Sponsor's product;
b. An elevated AST or ALT laboratory value that is greater than or equal to 3× the ULN and an elevated total bilirubin laboratory value that is greater than or equal to 2× the ULN and, at the same time, an alkaline phosphatase laboratory value that is less than 2× the ULN, as determined by way of protocol-specified laboratory testing or unscheduled laboratory testing. These criteria are based on available regulatory guidance documents. The purpose of the criteria is to specify a threshold of abnormal hepatic tests that may require an additional evaluation for an underlying etiology.

For the time period beginning when the consent form is signed until treatment allocation, any ECI, or follow-up to an ECI, that occurs to any participant must be reported within 24 hours to the Sponsor if it causes the participant to be excluded from the study, or is the result of a protocol-specified intervention, including but not limited to washout or discontinuation of usual therapy, diet, or a procedure.

### Treatment of Overdose

For purposes of this study, an overdose will be defined as any dose exceeding the prescribed dose for anti-ILT3 antibody by ≥20% of the indicated dose. No specific information is available on the treatment of overdose of anti-ILT3 antibody. In the event of overdose, anti-ILT3 antibody may be discontinued and the participant should be observed closely for signs of toxicity. Appropriate supportive treatment should be provided if clinically indicated.

### REFERENCES

1. [American Cancer Society 2021] American Cancer Society. Cancer facts and figures, 2021. Atlanta (GA): American Cancer Society (ACS); 2021. p. 17.
2. [Arber, D. A., et al. 2016] Arber D A et al. The 2016 revision to the World Health Organization classification of myeloid neoplasms and acute leukemia. Blood. 2016 May 19; 127(20):2391-405.
3. [Carter, J. L., et al. 2020] Carter J L et al. Targeting multiple signaling pathways: the new approach to acute myeloid leukemia therapy. Signal Transduct Target Ther. 2020; 5:288.
4. [Cella, M., et al. 1997] Cella M et al. A novel inhibitory receptor (ILT3) expressed on monocytes, macrophages, and dendritic cells involved in antigen processing. J Exp Med. 1997 May 19; 185(10):1743-51.
5. [Chien, K. S., et al. 2020] Chien K S, Class C A, Montalban-Bravo G, Wei Y, Sasaki K, Naqvi K, et al. LILRB4 expression in chronic myelomonocytic leukemia and myelodysplastic syndrome based on response to hypomethylating agents. Leuk Lymphoma. 2020 June; 61(6):1493-1499.
6. [Dohner, H., et al. 2015] Dohner H et al. Acute myeloid leukemia. N Engl J Med. 2015 Sep. 17; 373(12):1136-52.
7. [Dohner, H., et al. 2017] Dohner H et al. Diagnosis and management of AML in adults: 2017 ELN recommendations from an international expert panel. Blood. 2017 Jan. 26; 129(4):424-47.
8. [Geissler, K. 2021] Geissler K. Molecular pathogenesis of chronic myelomonocytic leukemia and potential molecular targets for treatment approaches. Front Oncol. 2021 Sep. 30; 11:751668.
9. [Gui, X., et al. 2019] Gui X et al. Disrupting LILRB4/APOE interaction by an efficacious humanized antibody reverses T-cell suppression and blocks AML development. Cancer Immunol Res. 2019 August; 7(8):1244-57.
10. [Howard. S. C., et al. 2011] Howard S C et al. The tumor lysis syndrome. N Engl J Med 2011; 364:1844-54.
11. [Ji Y et al. 2007] Ji Y et al. Dose-finding in phase 1 clinical trials based on toxicity probability intervals. Clin Trials 2007; 4:235-44.
12. [Ji, Y. et al. 2013] Ji Y et al. Modified toxicity probability interval design: a safer and more reliable method than the 3+3 design for practical phase I trials. J Clin Oncol 2013; 31:1-12.
13. [Ji, Y., et al. 2010] Ji Y et al. A modified toxicity probability interval method for dose-finding trials. Clin Trials. 2010; 7:653-63.
14. [Kang, X., et al. 2016] Kang X et al. Inhibitory leukocyte immunoglobulin-like receptors: immune checkpoint proteins and tumor sustaining factors. Cell Cycle.

2016; 15(1):25-40.
15. [Li, Z., et al. 2020] Li Z et al. LILRB4 ITIMs mediate the T cell suppression and infiltration of acute myeloid leukemia cells. Cell Mol Immunol. 2020; 17:272-82. Erratum in: Cell Mol Immunol. 2020; 17:302-4.
16. [National Comprehensive Cancer Network 2021] National Comprehensive Cancer Network. NCCN clinical practice guidelines in oncology: acute myeloid leukemia; version 3.2021. Plymouth Meeting (PA): National Comprehensive Cancer Network (NCCN); 2021.
17. [Orazi, A., et al. 2008] Orazi A et al. WHO classification of tumours of haematopoietic and lymphoid tissues. 4th ed. Swerdlow S H, Campo E, Harris N L, Jaffe E S. Pileri S A, Stein H, et al., editors. Lyon (France): International Agency for Research on Cancer (IARC); c2008. Chronic myelomonocytic leukaemia; p. 76-9.
18. [Patnaik, M. M. 2018] Patnaik M M, Tefferi A. Chronic myelomonocytic leukemia: 2018 update on diagnosis, risk stratification and management. Am J Hematol.

2018; 93:824-40.
19. [Shallis, R. M., et al. 2019] Shallis R M et al. Epidemiology of acute myeloid leukemia: recent progress and enduring challenges. Blood Rev. 2019; 36:70-87.
20. [Siegel, R. L., et al. 2021] Siegel R L et al. Cancer statistics, 2021. CA Cancer J Clin. 2021 January-February; 71(1):7-33.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A method for treating chronic myelomonocytic leukemia (CMML) in a human subject comprising administering to the subject a therapeutically effective dose of a pharmaceutical composition comprising an anti-ILT3 antigen binding protein or antigen binding fragment and a pharmaceutically acceptable excipient.

2. The method of claim 1, wherein the subject has a confirmed diagnosis of CMML.

3. The method of claim 1, wherein the subject has confirmed CMML with a) persistent monocytosis>1×10⁹/L in the peripheral blood with monocytes≥10% of white blood cell count; b) dysplastic features in the bone marrow, lack of Philadelphia chromosome and BCR-ABL 1 fusion gene; c) no rearrangement of PDGFRA or PDGFRB; d) fewer than 20% blasts in peripheral blood and bone marrow; and e) dysplasia involving one or more myeloid lineages.

4. (canceled)

5. The method of claim 1, wherein the anti-ILT3 antigen binding protein or antigen-binding fragment is an anti-ILT3 antibody or antigen-binding fragment.

6. (canceled)

7. The method of claim 5, wherein the anti-ILT3 antibody or antigen binding fragment comprises:
(a) a heavy chain (HC) having a variable domain (VH) comprising a variable domain complementarity determining region (HC-CDR) 1 having the amino acid sequence set forth in SEQ ID NO: 10, 40, 48, 56, 64, 72, 80, 88, or 96; an HC-CDR2 having the amino acid sequence set forth in SEQ ID NO: 11, 41, 48, 57, 64, 73, 81, 89, or 97; and an HC-CDR3 having the amino acid sequence set forth in SEQ ID NO: 16, 42, 50, 58, 66, 74, 82, 90, or 98; and, variants thereof wherein one or more of the HC-CDRs has one, two, or three amino acid substitutions, additions, deletions, or combinations thereof, and
(b) a light chain (LC) having variable domain (VL) comprising a variable domain complementarity determining region (LC-CDR) 1 having the amino acid sequence set forth in SEQ ID NO: 20, 43, 51, 59, 67, 75, 83, 91, or 99; an LC-CDR2 having the amino acid sequence set forth in SEQ ID NO: 36, 44, 52, 60, 68, 76, 84, 92, or 100; and an LC-CDR3 having the amino acid sequence set forth in SEQ ID NO: 37, 45, 53, 61, 69, 77, 85, 93, or 101; and, variants thereof wherein one or more of the LC-CDRs has one, two, or three amino acid substitutions, additions, deletions, or combinations thereof.

8. The method of claim 7, wherein:
(a) the HC-CDR1 has the amino acid sequence set forth in SEQ ID NO: 10; the HC-CDR2 has the amino acid sequence set forth in SEQ ID NO: 12, 13, or 14; the HC-CDR3 has the amino acid sequence set forth in SEQ ID NO: 16; and
(b) the LC-CDR1 has the amino acid sequence set forth in SEQ ID NO: 27, 28, 29, 30, 31, 32, 33, 34, or 35; the LC-CDR2 has the amino acid sequence set forth in SEQ ID NO: 36; and, the LC-CDR3 has the amino acid sequence set forth in SEQ ID NO: 37.

9. (canceled)

10. The method of claim 7, wherein the V_{H} comprises a framework selected from the group consisting of human V_{H}1, V_{H}2, V_{H}3, V_{H}4, V_{H}5, and V_{H}6, and variants thereof having 1, 2, 3, 4, 5, 6, 7, 8, 9, or 10 amino acid substitutions, additions, deletions, or combinations thereof, and, the V_{L} comprises a framework selected from the group consisting of human V_{K}1, V_{K}2, V_{K}3, V_{K}4, V_{K}5, V_{K}6, V_{λ}1, V_{λ}2, V_{λ}3, V_{λ}4, V_{λ}5, V_{λ}6, V_{λ}7, V_{λ}8, V_{λ}9, and V_{λ}10, and variants thereof having 1, 2, 3, 4, 5, 6, 7, 8, 9, or 10 amino acid substitutions, additions, deletions, or combinations thereof.

11. The method of claim 7, wherein the antibody comprises an HC having a human IgG1, IgG2, IgG3, or IgG4 HC constant domain or variant thereof having 1, 2, 3, 4, 5, 6, 7, 8, 9, or 10 amino acid substitutions, additions, deletions, or combinations thereof compared to the amino acid sequence of the native IgG1, IgG2, IgG3, or IgG4 isotype constant domain, and wherein the antibody comprises an LC having a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda light chain constant domain.

12. (canceled)

13. The method of claim 8, wherein the antibody comprises:
(i) a V_{H} having a framework selected from human V_{H}1, V_{H}2, V_{H}3, V_{H}4, VHS, and V_{H}6 and a human IgG1 or IgG4 HC constant domain or variant thereof comprising 1, 2, 3, 4, 5, 6, 7, 8, 9, or 10 amino acid substitutions, additions, deletions, or combinations thereof compared to the amino acid sequence of the native IgG1 or IgG4 isotype HC constant domain; and,
(ii) a V_{L} having a framework selected from human V_{K}1, V_{K}2, V_{K}3, V_{K}4, V_{K}5, V_{K}6, V_{λ}1, V_{λ}2, V_{λ}3, V_{λ}4, V_{λ}5, V_{λ}6, V_{λ}7, V_{λ}8, V_{λ}9, and V_{λ}10 and a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda LC constant domain.

14. (canceled)

15. The method of claim 7, wherein the antibody or antigen binding fragment comprises a V_{H} having the amino acid sequence set forth in SEQ ID NO: 110, 111, 112, 116, 117, or 118 and a V_{L} having the amino acid sequence set forth in SEQ ID NO: 119, 120, 121, 122, 123, 124, 125, 126, 127, 128, 129, 130, 131, 132, 133, or 134.

16-18. (canceled)

19. The method of claim 15, wherein the antibody comprises a heavy chain (HC) comprising the amino acid sequence of SEQ ID NO: 135, 136, 137, 141, 142, 143, 160, 161, 162, 163, 167, 168, 169, 170, 171, 175, 176, 177, 178, 179, 180, 184, 185, or 186, and wherein the antibody comprises a light chain (LC) comprising the amino acid sequence set forth in SEQ ID NO: 144, 145, 146, 147, 148, 149, 150, 151, 152, 153, 154, 155, 156, 157, 158, or 159.

20. (canceled)

21. The method of claim 13, wherein the antibody comprises a heavy chain (HC) comprising the amino acid sequence set forth in SEQ ID NO: 136 and a light chain (LC) comprising the amino acid sequence set forth in SEQ ID NO: 158, or a variant wherein the HC lacks a C-terminal Lysine residue or the HC lacks a C-terminal glycine-lysine.

22. The method of claim 7, wherein the therapeutically effective dose of the anti-ILT3 antibody or antigen binding fragment is between about 7.5 mg and about 2250 mg.

23. The method of claim 7, wherein the therapeutically effective dose of anti-ILT3 antibody or antigen binding fragment is selected from the group consisting of: 7.5 mg; 25 mg; 75 mg; 225 mg; 750 mg; and 2250 mg.

24-29. (canceled)

30. The method of claim 22, wherein the anti-ILT3 antibody or antigen binding fragment is administered every three weeks (Q3W) of a 21-day cycle.

31-34. (canceled)

35. The method of claim 30, wherein the anti-ILT3 antibody or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 10; the HC-CDR2 has the amino acid sequence set forth in SEQ ID NO: 13; the HC-CDR3 has the amino acid sequence set forth in SEQ ID NO: 16; the LC-CDR1 has the amino acid sequence set forth in SEQ ID NO: 34; the LC-CDR2 has the amino acid sequence set forth in SEQ ID NO: 36; and, the LC-CDR3 has the amino acid sequence set forth in SEQ ID NO: 37.

36-42. (canceled)

43. A pharmaceutical composition comprising 0.02 mg to 2250 mg of an anti-ILT3 antigen binding protein or antigen binding fragment and a pharmaceutically acceptable excipient.

44. (canceled)

45. The method of claim 23, wherein the anti-ILT3 antibody or antigen binding fragment is administered every three weeks (Q3W) of a 21-day cycle.

46. The method of claim 45, wherein the anti-ILT3 antibody or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 10; the HC-CDR2 has the amino acid sequence set forth in SEQ ID NO: 13; the HC-CDR3 has the amino acid sequence set forth in SEQ ID NO: 16; the LC-CDR1 has the amino acid sequence set forth in SEQ ID NO: 34; the LC-CDR2 has the amino acid sequence set forth in SEQ ID NO: 36; and, the LC-CDR3 has the amino acid sequence set forth in SEQ ID NO: 37.

47. The pharmaceutical composition of claim 43, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 10; the HC-CDR2 has the amino acid sequence set forth in SEQ ID NO: 13; the HC-CDR3 has the amino acid sequence set forth in SEQ ID NO: 16; the LC-CDR1 has the amino acid sequence set forth in SEQ ID NO: 34; the LC-CDR2 has the amino acid sequence set forth in SEQ ID NO: 36; and, the LC-CDR3 has the amino acid sequence set forth in SEQ ID NO: 37.


SEQUENCE LISTING (253 sequences)

SEQ ID NO: 1 - protein, 238 aa, Homo sapiens
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE

SEQ ID NO: 2 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 330 aa, Homo sapiens [sequence omitted]

SEQ ID NO: 5 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 107 aa, Homo sapiens [sequence omitted]

SEQ ID NO: 8 - protein, 122 aa, synthetic construct
EVQLVESGGDLVKPGGSLKLSCAASGFTFSNYGMSWVRQTPDRRLEWVATISGGGDYTNYPDSMRGRFTISRDNAKNTLYLQMSSLKSEDTAMYYCGRRLWFRSLYYAMDYWGQGTSVTVSS

SEQ ID NO: 9 - protein, 111 aa, synthetic construct
NIVLTQSPASLAVSLGQRATISCRASEKVDSFGNSFMHWYQQKPGQPPKLLIYLTSNLDSGVPARFSGSGSRTDFALTIDPVEADDAATYYCQQNNEDPYTFGGGTKLEIK

SEQ ID NO: 10 - protein, 5 aa, synthetic construct
NYGMS

SEQ ID NO: 11 - protein, 17 aa, synthetic construct
  VARIANT 15: note = M, V, or L
TISGGGDYTNYPDSXRG

SEQ ID NO: 12 - protein, 17 aa, synthetic construct
TISGGGDYTNYPDSMRG

SEQ ID NO: 13 - protein, 17 aa, synthetic construct
TISGGGDYTNYPDSVRG

SEQ ID NO: 14 - protein, 17 aa, synthetic construct
TISGGGDYTNYPDSLRG

SEQ ID NO: 15 - protein, 13 aa, synthetic construct
  VARIANT 3: note = W, Y, Q, or F
RLXFRSLYYAMDY

SEQ ID NO: 16 - protein, 13 aa, synthetic construct
RLWFRSLYYAMDY

SEQ ID NO: 17 - protein, 13 aa, synthetic construct
RLYFRSLYYAMDY

SEQ ID NO: 18 - protein, 13 aa, synthetic construct
RLQFRSLYYAMDY

SEQ ID NO: 19 - protein, 13 aa, synthetic construct
RLFFRSLYYAMDY

SEQ ID NO: 20 - protein, 15 aa, synthetic construct
  VARIANT 11: note = N, D, or Q
  VARIANT 12: note = S, N, or A
RASEKVDSFGXXFMH

SEQ ID NO: 21 - protein, 15 aa, synthetic construct
  VARIANT 12: note = S, N, or A
RASEKVDSFGNXFMH

SEQ ID NO: 22 - protein, 15 aa, synthetic construct
  VARIANT 12: note = S, N, or A
RASEKVDSFGDXFMH

SEQ ID NO: 23 - protein, 15 aa, synthetic construct
  VARIANT 12: note = S, N, or A
RASEKVDSFGQXFMH

SEQ ID NO: 24 - protein, 15 aa, synthetic construct
  VARIANT 11: note = N, D, or Q
RASEKVDSFGXSFMH

SEQ ID NO: 25 - protein, 15 aa, synthetic construct
  VARIANT 11: note = N, D, or Q
RASEKVDSFGXNFMH

SEQ ID NO: 26 - protein, 15 aa, synthetic construct
  VARIANT 11: note = N, D, or Q
RASEKVDSFGXAFMH

SEQ ID NO: 27 - protein, 15 aa, synthetic construct
RASEKVDSFGNNFMH

SEQ ID NO: 28 - protein, 15 aa, synthetic construct
RASEKVDSFGDNFMH

SEQ ID NO: 29 - protein, 15 aa, synthetic construct
RASEKVDSFGQNFMH

SEQ ID NO: 30 - protein, 15 aa, synthetic construct
RASEKVDSFGNSFMH

SEQ ID NO: 31 - protein, 15 aa, synthetic construct
RASEKVDSFGDSFMH

SEQ ID NO: 32 - protein, 15 aa, synthetic construct
RASEKVDSFGNAFMH

SEQ ID NO: 33 - protein, 15 aa, synthetic construct
RASEKVDSFGDAFMH

SEQ ID NO: 34 - protein, 15 aa, synthetic construct
RASEKVDSFGQSFMH

SEQ ID NO: 35 - protein, 15 aa, synthetic construct
RASEKVDSFGQAFMH

SEQ ID NO: 36 - protein, 7 aa, synthetic construct
LTSNLDS

SEQ ID NO: 37 - protein, 9 aa, synthetic construct
QQNNEDPYT

SEQ ID NO: 38 - protein, 121 aa, synthetic construct
QVQLKESGPGLVQASETLSLTCTVSGFSLTSYSINWVRQSSGKGPEWMGRFWYDEGIAYNLTLESRLSISGDTSKNQVFLKMNSLRTGDTGTYYCTRDRDTVGITGWFAYWGQGTLVTVSS

SEQ ID NO: 39 - protein, 107 aa, synthetic construct
ETVMTQSPTSLSASIGERVTLNCKASQSVGVNVDWYQQTPGQSPKLLIYGSANRHTGVPDRFTGSGFGSDFTLTISDVEPEDLGVYYCLQYGSVPYTFGAGTKLELK

SEQ ID NO: 40 - protein, 5 aa, synthetic construct
SYSIN

SEQ ID NO: 41 - protein, 16 aa, synthetic construct
RFWYDEGIAYNLTLES

SEQ ID NO: 42 - protein, 13 aa, synthetic construct
DRDTVGITGWFAY

SEQ ID NO: 43 - protein, 11 aa, synthetic construct
KASQSVGVNVD

SEQ ID NO: 44 - protein, 7 aa, synthetic construct
GSANRHT

SEQ ID NO: 45 - protein, 9 aa, synthetic construct
LQYGSVPYT

SEQ ID NO: 46 - protein, 121 aa, synthetic construct
QVQLKESGPGLVQASETLSLTCTVSGFSLTNYCVNWVRQPSGKGPEWLGRFWFDEGKAYNLTLESRLSISGDTSKNQVFLRMNSLRADDTGTYYCTRDRDTVGITGWFAYWGQGTLVTVSS

SEQ ID NO: 47 - protein, 107 aa, synthetic construct
ETVMTQSPTSLSASIGERVTLNCKASQSVGINVDWYQQTPGQSPKLLIYGSANRHTGVPDRFTGSGFGSDFTLTISNVEPEDLGVYYCLQYGSVPYTFGPGTKLELK

SEQ ID NO: 48 - protein, 5 aa, synthetic construct
NYCVN

SEQ ID NO: 49 - protein, 16 aa, synthetic construct
RFWFDEGKAYNLTLES

SEQ ID NO: 50 - protein, 13 aa, synthetic construct
DRDTVGITGWFAY

SEQ ID NO: 51 - protein, 11 aa, synthetic construct
KASQSVGINVD

SEQ ID NO: 52 - protein, 7 aa, synthetic construct
GSANRHT

SEQ ID NO: 53 - protein, 9 aa, synthetic construct
LQYGSVPYT

SEQ ID NO: 54 - protein, 118 aa, synthetic construct
QVQLQQSGAELMKPGASVKISCKATGYTFRTYWIEWVKQRPGHGLEWIGEILPGNGNTHFNENFKDKATFTADTSSNAAYMQLSSLTSEDSAVYYCVRRLGRGPFDFWGQGTTLTVSS

SEQ ID NO: 55 - protein, 107 aa, synthetic construct
DIQMTQSPSSLSVSLGGKVTITCKASQDINEYIGWYQRKPGKGPRLLIHYTSTLQSGIPSRFSGSGSGRDYSLSISNLEPEDIATYYCLQYANPLPTFGGGTKLEIK

SEQ ID NO: 56 - protein, 5 aa, synthetic construct
TYWIE

SEQ ID NO: 57 - protein, 17 aa, synthetic construct
EILPGNGNTHFNENFKD

SEQ ID NO: 58 - protein, 10 aa, synthetic construct
RRLGRGPFDF

SEQ ID NO: 59 - protein, 11 aa, synthetic construct
KASQDINEYIG

SEQ ID NO: 60 - protein, 7 aa, synthetic construct
YTSTLQS

SEQ ID NO: 61 - protein, 9 aa, synthetic construct
LQYANPLPT

SEQ ID NO: 62 - protein, 122 aa, synthetic construct
EVQLVESGGGLVQPGRSMKLSCAASGFTFSNFDMAWVRQAPTRGLEWVSSITYDGGSTSYRDSVKGRFTISRDNAKGTLYLQMDSLRSEDTATYYCTTVESIATISTYFDYWGQGVMVTVSS

SEQ ID NO: 63 - protein, 110 aa, synthetic construct
DIVLTQSPALAVSLGQRATISCRASQSVSMSRYDLIHWYQQKPGQQPKLLIFRASDLASGIPARFSGSGSGTDFTLTINPVQADDIATYYCQQTRKSPPTFGGGTRLELK

SEQ ID NO: 64 - protein, 5 aa, synthetic construct
NFDMA

SEQ ID NO: 65 - protein, 17 aa, synthetic construct
SITYDGGSTSYRDSVKG

SEQ ID NO: 66 - protein, 13 aa, synthetic construct
VESIATISTYFDY

SEQ ID NO: 67 - protein, 15 aa, synthetic construct
RASQSVSMSRYDLIH

SEQ ID NO: 68 - protein, 7 aa, synthetic construct
RASDLAS

SEQ ID NO: 69 - protein, 9 aa, synthetic construct
QQTRKSPPT

SEQ ID NO: 70 - protein, 121 aa, synthetic construct
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTMGITGWFAYWGQGTLVTVSS

SEQ ID NO: 71 - protein, 107 aa, synthetic construct
ETVMTQSPTSLSASIGERVTLNCKASQSVGINVDWYQQTPGQSPKLLIYGSANRHTGVPDRFTGSGFGSGFTLTISNVEPEDLGVYYCLQYGSVPYTFGPGTKLELK

SEQ ID NO: 72 - protein, 5 aa, synthetic construct
SYCVN

SEQ ID NO: 73 - protein, 16 aa, synthetic construct
RFWYDEGKVYNLTLES

SEQ ID NO: 74 - protein, 13 aa, synthetic construct
DRDTMGITGWFAY

SEQ ID NO: 75 - protein, 11 aa, synthetic construct
KASQSVGINVD

SEQ ID NO: 76 - protein, 7 aa, synthetic construct
GSANRHT

SEQ ID NO: 77 - protein, 9 aa, synthetic construct
LQYGSVPYT

SEQ ID NO: 78 - protein, 118 aa, synthetic construct
QVQMQQSGTELMKPGASMKISCKATGYTFSTYWIQWIKQRPGHGLEWIGEILPGSGTTNYNENFKGKATFSADTSSNTAYIHLSSLTSEDSAVFYCARRLGRGPFDYWGQGTTLTVSS

SEQ ID NO: 79 - protein, 107 aa, synthetic construct
DIQMTQSPSSLSASLGGKVTITCEASQDINKHIDWYQHQPGRGPSLLIHYASILQPGIPSRFSGSGSGRDYSFSITSLEPEDIATYYCLQYDNLLPTFGGGTKLEIK

SEQ ID NO: 80 - protein, 5 aa, synthetic construct
TYWIQ

SEQ ID NO: 81 - protein, 17 aa, synthetic construct
EILPGSGTTNYNENFKG

SEQ ID NO: 82 - protein, 9 aa, synthetic construct
RLGRGPFDY

SEQ ID NO: 83 - protein, 11 aa, synthetic construct
EASQDINKHID

SEQ ID NO: 84 - protein, 7 aa, synthetic construct
YASILQP

SEQ ID NO: 85 - protein, 9 aa, synthetic construct
LQYDNLLPT

SEQ ID NO: 86 - protein, 121 aa, synthetic construct
QVQLKESGPGLVQASETLSLTCTVSGFSLTSYSVNWVRQPSGKGLEWMGRFWYDGGTAYNSTLESRLSISGDTSKNQVFLKMNSLQTDDTGTYYCTRDRDTMGITGWFAYWGQGTLVTVSP

SEQ ID NO: 87 - protein, 107 aa, synthetic construct
ETVMTQSPTSLSASIGERVTLNCKASQSVGVNVDWYQQTPGQSPKLLIYGSANRHTGVPDRFTGSGFGSDFTLTISNVEPEDLGVYYCLQYGSVPYTFGAGTKLELK

SEQ ID NO: 88 - protein, 5 aa, synthetic construct
SYSVN

SEQ ID NO: 89 - protein, 16 aa, synthetic construct
RFWYDGGTAYNSTLES

SEQ ID NO: 90 - protein, 13 aa, synthetic construct
DRDTMGITGWFAY

SEQ ID NO: 91 - protein, 11 aa, synthetic construct
KASQSVGVNVD

SEQ ID NO: 92 - protein, 7 aa, synthetic construct
GSANRHT

SEQ ID NO: 93 - protein, 9 aa, synthetic construct
LQYGSVPYT

SEQ ID NO: 94 - protein, 121 aa, synthetic construct
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTLGITGWFAYWGQGTLVTVSS

SEQ ID NO: 95 - protein, 107 aa, synthetic construct; identical to SEQ ID NO: 71

SEQ ID NO: 96 - protein, 5 aa, synthetic construct
SYCVN

SEQ ID NO: 97 - protein, 16 aa, synthetic construct
RFWYDEGKVYNLTLES

SEQ ID NO: 98 - protein, 13 aa, synthetic construct
DRDTLGITGWFAY

SEQ ID NO: 99 - protein, 11 aa, synthetic construct
KASQSVGINVD

SEQ ID NO: 100 - protein, 7 aa, synthetic construct
GSANRHT

SEQ ID NO: 101 - protein, 9 aa, synthetic construct
LQYGSVPYT

SEQ ID NO: 102 - protein, 19 aa, unidentified
MEWSWVFLFFLSVTTGVHS

SEQ ID NO: 103 - protein, 20 aa, unidentified
MSVPTQVLGLLLLWLTDARC

SEQ ID NO: 104 - protein, 452 aa, synthetic construct
EVQLVESGGDLVKPGGSLKLSCAASGFTFSNYGMSWVRQTPDRRLEWVATISGGGDYTNYPDSMRGRFTISRDNAKNTLYLQMSSLKSEDTAMYYCGRRLWFRSLYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 105 - protein, 218 aa, synthetic construct
NIVLTQSPASLAVSLGQRATISCRASEKVDSFGNSFMHWYQQKPGQPPKLLIYLTSNLDSGVPARFSGSGSRTDFALTIDPVEADDAATYYCQQNNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 106 - protein, 449 aa, synthetic construct
EVQLVESGGDLVKPGGSLKLSCAASGFTFSNYGMSWVRQTPDRRLEWVATISGGGDYTNYPDSMRGRFTISRDNAKNTLYLQMSSLKSEDTAMYYCGRRLWFRSLYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 449 aa, synthetic construct
EVQLVESGGDLVKPGGSLKLSCAASGFTFSNYGMSWVRQTPDRRLEWVATISGGGDYTNYPDSVRGRFTISRDNAKNTLYLQMSSLKSEDTAMYYCGRRLWFRSLYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 449 aa, synthetic construct
EVQLVESGGDLVKPGGSLKLSCAASGFTFSNYGMSWVRQTPDRRLEWVATISGGGDYTNYPDSLRGRFTISRDNAKNTLYLQMSSLKSEDTAMYYCGRRLWFRSLYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 218 aa, synthetic construct
NIVLTQSPASLAVSLGQRATISCRASEKVDSFGNSFMHWYQQKPGQPPKLLIYLTSNLDSGVPARFSGSGSRTDFALTIDPVEADDAATYYCQQNNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 122 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSS

SEQ ID NO: 111 - protein, 122 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSS

SEQ ID NO: 112 - protein, 122 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSS

SEQ ID NO: 113 - protein, 122 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSS

SEQ ID NO: 114 - protein, 122 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSS

SEQ ID NO: 115 - protein, 122 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSS

SEQ ID NO: 116 - protein, 122 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSS

SEQ ID NO: 117 - protein, 122 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSS

SEQ ID NO: 118 - protein, 122 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSS

SEQ ID NO: 119 - protein, 111 aa, synthetic construct
DIVLTQSPDSLAVSLGERATINCRASEKVDSFGNSFMHWYQQKPGQPPKLLIYLTSNLDSGVPDRFSGSGSRTDFTLTISSLQAEDVAVYYCQQNNEDPYTFGQGTKLEIK

SEQ ID NO: 120 - protein, 111 aa, synthetic construct
DIVLTQSPDSLAVSLGERATINCRASEKVDSFGNSFMHWYQQKPGQPPKLLIYLTSNLDSGVPDRFSGSGSGTDFTLTISSLQAEDVAVYYCQQNNEDPYTFGQGTKLEIK

SEQ ID NO: 121 - protein, 111 aa, synthetic construct
EIVLTQSPATLSLSPGERATLSCRASEKVDSFGNSFMHWYQQKPGQAPRLLIYLTSNLDSGVPARFSGSGSRTDFTLTISSLEPEDFAVYYCQQNNEDPYTFGQGTKLEIK

SEQ ID NO: 122 - protein, 111 aa, synthetic construct
EIVLTQSPATLSLSPGERATLSCRASEKVDSFGNSFMHWYQQKPGQAPRLLIYLTSNLDSGIPARFSGSGSGTDFTLTISSLEPEDFAVYYCQQNNEDPYTFGQGTKLEIK

SEQ ID NO: 123 - protein, 111 aa, synthetic construct
DIQLTQSPSSLSASVGDRVTITCRASEKVDSFGNSFMHWYQQKPGKAPKLLIYLTSNLDSGVPSRFSGSGSGTDFTLTISSLQPEDFATYYCQQNNEDPYTFGQGTKLEIK

SEQ ID NO: 124 - protein, 111 aa, synthetic construct
DIQMTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIK

SEQ ID NO: 125 - protein, 111 aa, synthetic construct
DIQLTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIK

SEQ ID NO: 126 - protein, 111 aa, synthetic construct
DIQLTQSPSSLSASVGDRVTITCRASEKVDSFGNSFMHWYQQKPGKAPKLLIYLTSNLDSGVPARFSGSGSRTDFTLTISSLQPEDFATYYCQQNNEDPYTFGQGTKLEIK

SEQ ID NO: 127 - protein, 111 aa, synthetic construct
DIVLTQSPDSLAVSLGERATINCRASEKVDSFGNAFMHWYQQKPGQPPKLLIYLTSNLDSGVPDRFSGSGSGTDFTLTISSLQAEDVAVYYCQQNNEDPYTFGQGTKLEIK

SEQ ID NO: 128 - protein, 111 aa, synthetic construct
DIVLTQSPDSLAVSLGERATINCRASEKVDSFGNNFMHWYQQKPGQPPKLLIYLTSNLDSGVPDRFSGSGSGTDFTLTISSLQAEDVAVYYCQQNNEDPYTFGQGTKLEIK

SEQ ID NO: 129 - protein, 111 aa, synthetic construct
DIVLTQSPDSLAVSLGERATINCRASEKVDSFGQSFMHWYQQKPGQPPKLLIYLTSNLDSGVPDRFSGSGSGTDFTLTISSLQAEDVAVYYCQQNNEDPYTFGQGTKLEIK

SEQ ID NO: 130 - protein, 111 aa, synthetic construct
DIVLTQSPDSLAVSLGERATINCRASEKVDSFGDSFMHWYQQKPGQPPKLLIYLTSNLDSGVPDRFSGSGSGTDFTLTISSLQAEDVAVYYCQQNNEDPYTFGQGTKLEIK

SEQ ID NO: 131 - protein, 111 aa, synthetic construct
DIQLTQSPSSLSASVGDRVTITCRASEKVDSFGNAFMHWYQQKPGKAPKLLIYLTSNLDSGVPSRFSGSGSGTDFTLTISSLQPEDFATYYCQQNNEDPYTFGQGTKLEIK

SEQ ID NO: 132 - protein, 111 aa, synthetic construct
DIQLTQSPSSLSASVGDRVTITCRASEKVDSFGNNFMHWYQQKPGKAPKLLIYLTSNLDSGVPSRFSGSGSGTDFTLTISSLQPEDFATYYCQQNNEDPYTFGQGTKLEIK

SEQ ID NO: 133 - protein, 111 aa, synthetic construct
DIQLTQSPSSLSASVGDRVTITCRASEKVDSFGQSFMHWYQQKPGKAPKLLIYLTSNLDSGVPSRFSGSGSGTDFTLTISSLQPEDFATYYCQQNNEDPYTFGQGTKLEIK

SEQ ID NO: 134 - protein, 111 aa, synthetic construct
DIQLTQSPSSLSASVGDRVTITCRASEKVDSFGDSFMHWYQQKPGKAPKLLIYLTSNLDSGVPSRFSGSGSGTDFTLTISSLQPEDFATYYCQQNNEDPYTFGQGTKLEIK

SEQ ID NO: 135 - protein, 449 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 449 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 137 - protein, 449 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 449 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - protein, 449 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 449 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 449 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 449 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - protein, 449 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 218 aa, synthetic construct
DIVLTQSPDSLAVSLGERATINCRASEKVDSFGNSFMHWYQQKPGQPPKLLIYLTSNLDSGVPDRFSGSGSRTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 218 aa, synthetic construct
DIVLTQSPDSLAVSLGERATINCRASEKVDSFGNSFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 218 aa, synthetic construct
EIVLTQSPATLSLSPGERATLSCRASEKVDSFGNSFMHWYQQKPGQAPRLLIYLTSNLDSGVPARFSGSGSRTDFTLTISSLEPEDFAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 218 aa, synthetic construct
EIVLTQSPATLSLSPGERATLSCRASEKVDSFGNSFMHWYQQKPGQAPRLLIYLTSNLDSGIPARFSGSGSGTDFTLTISSLEPEDFAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 218 aa, synthetic construct
DIQLTQSPSSLSASVGDRVTITCRASEKVDSFGNSFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 218 aa, synthetic construct
DIQMTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 218 aa, synthetic construct
DIQLTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 218 aa, synthetic construct
DIQLTQSPSSLSASVGDRVTITCRASEKVDSFGNSFMHWYQQKPGKAPKLLIYLTSNLDSGVPARFSGSGSRTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 218 aa, synthetic construct
DIVLTQSPDSLAVSLGERATINCRASEKVDSFGNAFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 218 aa, synthetic construct
DIVLTQSPDSLAVSLGERATINCRASEKVDSFGNNFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 218 aa, synthetic construct
DIVLTQSPDSLAVSLGERATINCRASEKVDSFGQSFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 218 aa, synthetic construct
DIVLTQSPDSLAVSLGERATINCRASEKVDSFGDSFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 218 aa, synthetic construct
DIQLTQSPSSLSASVGDRVTITCRASEKVDSFGNAFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 218 aa, synthetic construct
DIQLTQSPSSLSASVGDRVTITCRASEKVDSFGNNFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 218 aa, synthetic construct
DIQLTQSPSSLSASVGDRVTITCRASEKVDSFGQSFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 218 aa, synthetic construct
DIQLTQSPSSLSASVGDRVTITCRASEKVDSFGDSFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 452 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 452 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 452 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 452 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 452 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 452 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 452 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 452 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 452 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 448 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 170 - protein, 448 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 171 - protein, 448 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 172 - protein, 448 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 173 - protein, 448 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 174 - protein, 448 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 175 - protein, 448 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 176 - protein, 448 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 177 - protein, 448 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 178 - protein, 451 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 451 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - protein, 451 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - protein, 451 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 451 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 183 - protein, 451 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - protein, 451 aa, synthetic construct
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - protein, 451 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 186 - protein, 451 aa, synthetic construct
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 451 aa, synthetic construct
QVQLKESGPGLVQASETLSLTCTVSGFSLTSYSINWVRQSSGKGPEWMGRFWYDEGIAYNLTLESRLSISGDTSKNQVFLKMNSLRTGDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 214 aa, synthetic construct
ETVMTQSPTSLSASIGERVTLNCKASQSVGVNVDWYQQTPGQSPKLLIYGSANRHTGVPDRFTGSGFGSDFTLTISDVEPEDLGVYYCLQYGSVPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 451 aa, synthetic construct
QVQLKESGPGLVQASETLSLTCTVSGFSLTNYCVNWVRQPSGKGPEWLGRFWFDEGKAYNLTLESRLSISGDTSKNQVFLRMNSLRADDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 214 aa, synthetic construct
ETVMTQSPTSLSASIGERVTLNCKASQSVGINVDWYQQTPGQSPKLLIYGSANRHTGVPDRFTGSGFGSDFTLTISNVEPEDLGVYYCLQYGSVPYTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 448 aa, synthetic construct
QVQLQQSGAELMKPGASVKISCKATGYTFRTYWIEWVKQRPGHGLEWIGEILPGNGNTHFNENFKDKATFTADTSSNAAYMQLSSLTSEDSAVYYCVRRLGRGPFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 214 aa, synthetic construct
DIQMTQSPSSLSVSLGGKVTITCKASQDINEYIGWYQRKPGKGPRLLIHYTSTLQSGIPSRFSGSGSGRDYSLSISNLEPEDIATYYCLQYANPLPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 452 aa, synthetic construct
EVQLVESGGGLVQPGRSMKLSCAASGFTFSNFDMAWVRQAPTRGLEWVSSITYDGGSTSYRDSVKGRFTISRDNAKGTLYLQMDSLRSEDTATYYCTTVESIATISTYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 217 aa, synthetic construct
DIVLTQSPALAVSLGQRATISCRASQSVSMSRYDLIHWYQQKPGQQPKLLIFRASDLASGIPARFSGSGSGTDFTLTINPVQADDIATYYCQQTRKSPPTFGGGTRLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 451 aa, synthetic construct
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTMGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 214 aa, synthetic construct
ETVMTQSPTSLSASIGERVTLNCKASQSVGINVDWYQQTPGQSPKLLIYGSANRHTGVPDRFTGSGFGSGFTLTISNVEPEDLGVYYCLQYGSVPYTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 448 aa, synthetic construct
QVQMQQSGTELMKPGASMKISCKATGYTFSTYWIQWIKQRPGHGLEWIGEILPGSGTTNYNENFKGKATFSADTSSNTAYIHLSSLTSEDSAVFYCARRLGRGPFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 214 aa, synthetic construct
DIQMTQSPSSLSASLGGKVTITCEASQDINKHIDWYQHQPGRGPSLLIHYASILQPGIPSRFSGSGSGRDYSFSITSLEPEDIATYYCLQYDNLLPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 451 aa, synthetic construct
QVQLKESGPGLVQASETLSLTCTVSGFSLTSYSVNWVRQPSGKGLEWMGRFWYDGGTAYNSTLESRLSISGDTSKNQVFLKMNSLQTDDTGTYYCTRDRDTMGITGWFAYWGQGTLVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 214 aa, synthetic construct
ETVMTQSPTSLSASIGERVTLNCKASQSVGVNVDWYQQTPGQSPKLLIYGSANRHTGVPDRFTGSGFGSDFTLTISNVEPEDLGVYYCLQYGSVPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 451 aa, synthetic construct
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTLGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 214 aa, synthetic construct; identical to SEQ ID NO: 196

SEQ ID NO: 203 - protein, 452 aa, synthetic construct
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 451 aa, synthetic construct
QVQLKESGPGLVQASETLSLTCTVSGFSLTSYSINWVRQSSGKGPEWMGRFWYDEGIAYNLTLESRLSISGDTSKNQVFLKMNSLRTGDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 451 aa, synthetic construct
QVQLKESGPGLVQASETLSLTCTVSGFSLTNYCVNWVRQPSGKGPEWLGRFWFDEGKAYNLTLESRLSISGDTSKNQVFLRMNSLRADDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 448 aa, synthetic construct
QVQLQQSGAELMKPGASVKISCKATGYTFRTYWIEWVKQRPGHGLEWIGEILPGNGNTHFNENFKDKATFTADTSSNAAYMQLSSLTSEDSAVYYCVRRLGRGPFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 452 aa, synthetic construct
EVQLVESGGGLVQPGRSMKLSCAASGFTFSNFDMAWVRQAPTRGLEWVSSITYDGGSTSYRDSVKGRFTISRDNAKGTLYLQMDSLRSEDTATYYCTTVESIATISTYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 451 aa, synthetic construct
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTMGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 448 aa, synthetic construct
QVQMQQSGTELMKPGASMKISCKATGYTFSTYWIQWIKQRPGHGLEWIGEILPGSGTTNYNENFKGKATFSADTSSNTAYIHLSSLTSEDSAVFYCARRLGRGPFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 451 aa, synthetic construct
QVQLKESGPGLVQASETLSLTCTVSGFSLTSYSVNWVRQPSGKGLEWMGRFWYDGGTAYNSTLESRLSISGDTSKNQVFLKMNSLQTDDTGTYYCTRDRDTMGITGWFAYWGQGTLVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 451 aa, synthetic construct
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTLGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 451 aa, synthetic construct; identical to SEQ ID NO: 205

SEQ ID NO: 214-215 - skipped (2 entries)

SEQ ID NO: 216 - protein, 5 aa, synthetic construct
LEWIG

SEQ ID NO: 217 - skipped

SEQ ID NO: 218 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219-220 - skipped (2 entries)

SEQ ID NO: 221 - protein, 5 aa, synthetic construct
LEWIG

SEQ ID NO: 222 - skipped

SEQ ID NO: 223 - protein, 330 aa, synthetic construct; identical to SEQ ID NO: 218

SEQ ID NO: 224 - protein, 15 aa, synthetic construct
RASKGVSTSGYSYLH

SEQ ID NO: 225 - protein, 7 aa, synthetic construct
LASYLES

SEQ ID NO: 226 - protein, 9 aa, synthetic construct
QHSRDLPLT

SEQ ID NO: 227 - protein, 5 aa, synthetic construct
NYYMY

SEQ ID NO: 228 - protein, 17 aa, synthetic construct
GINPSNGGTNFNEKFKN

SEQ ID NO: 229 - protein, 11 aa, synthetic construct
RDYRFDMGFDY

SEQ ID NO: 230 - protein, 15 aa, synthetic construct
RASKSVSTSGFSYLH

SEQ ID NO: 231 - protein, 7 aa, synthetic construct
LASNLES

SEQ ID NO: 232 - protein, 9 aa, synthetic construct
QHSWELPLT

SEQ ID NO: 233 - protein, 5 aa, synthetic construct
SYYLY

SEQ ID NO: 234 - protein, 17 aa, synthetic construct
GVNPSNGGTNFSEKFKS

SEQ ID NO: 235 - protein, 11 aa, synthetic construct
RDSNYDGGFDY

SEQ ID NO: 236 - protein, 120 aa, synthetic construct [sequence omitted]

SEQ ID NO: 237 - protein, 111 aa, synthetic construct [sequence omitted]

SEQ ID NO: 238 - protein, 111 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 239 - protein, 111 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 240 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 241 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 218 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 218 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 11 aa, synthetic construct
RASQSVSSYLA

SEQ ID NO: 245 - protein, 7 aa, synthetic construct
DASNRAT

SEQ ID NO: 246 - protein, 9 aa, synthetic construct
QQSSNWPRT

SEQ ID NO: 247 - protein, 107 aa, synthetic construct [sequence omitted]

SEQ ID NO: 248 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 5 aa, synthetic construct
NSGMH

SEQ ID NO: 250 - protein, 17 aa, synthetic construct
VIWYDGSKRYYADSVKG

SEQ ID NO: 251 - protein, 4 aa, synthetic construct
NDDY

SEQ ID NO: 252 - protein, 113 aa, synthetic construct [sequence omitted]

SEQ ID NO: 253 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK